SEQUENCE LISTING (97 sequences)

SEQ ID NO: 1 - DNA, 25 bp, human
cattattcaaggccgagtacagatg

SEQ ID NO: 2 - DNA, 23 bp, human
cacgtacacgatgtgtcccctct

SEQ ID NO: 3 - DNA, 23 bp, human
caggcggtgtgcctgctgcattt

SEQ ID NO: 4 - DNA, 22 bp, human
gtcccggcgggattctgatgtt

SEQ ID NO: 5 - DNA, 112 bp, human
gaattcgcccttgagaaaacgacgcatccactactgcgattaccctggttgcacaaaagtttacaccaagtcttctcatttaaaagctcacctgaggactaagggcgaattc

SEQ ID NO: 6 - DNA, 60 bp, human
aaacgacgcatccactactgcgattaccctggttgcacaaaagtttacaccaagtcttct

SEQ ID NO: 7 - DNA, 1924 bp, human
ccatgacgcccgccctcacagccctgctctgccttgggctgagtctgggccccaggacccgcatgcaggcagggcccttccccaaacccaccctctgggctgagccaggctctgtgatcagctgggggagccccgtgaccatctggtgtcaggggagcctggaggcccaggagtaccaactggataaagagggaagcccagagccctgggacagaaataacccactggaacccaagaacaaggccagattctccatcccatccatgacacagcaccatgcagggagataccgctgccactattacagctctgcaggctggtcagagcccagcgaccccctggagctggtgatgacaggattctacaacaaacccaccctctcagccctgcccagccctgtggtggcctcaggggggaatatgaccctccgatgtggctcacagaagggatatcaccattttgttctgatgaaggaaggagaacaccagctcccccggaccctggactcacagcagctccacagtggggggttccaggccctgttccctgtgggccccgtgacccccagccacaggcgtgtctaggaagccctccctcctgaccctgcagggccctgtcctggcccctgggcagagcctgaccctccagtgtggctctgatgtcggctacgacagatttgttctgtataaggagggggaacgtgacttcctccagcgccctggccagcagccccaggctgggctctcccaggccaacttcaccctgggccctgtgagccgctcctacgggggccagtacaggtgctatggtgcacacaacctctcctccgagtggtcggcccccagtgaccccctggacatcctgatcacaggacagatctatgacaccgtctccctgtcagcacagccgggccccacagtggcctcaggagagaacatgaccctgctgtgtcagtcacgggggtattttgacactttccttctgaccaaagaaggggcagcccatcccccactgcgtctgagatcaatgtacggagctcataagtaccaggctgaattccccatgagtcctgtgacctcagcccacgcggggacctacaggtgctacggctcacgcagctccaacccccacctgctgtctttccccagtgagcccctggaactcatggtctcaggacactctggaggctccagcctcccacccacagggccgccctccacacctggtctgggaagatacctggaggttttgattggggtctcggtggccttcgtcctgctgctcttcctcctcctcttcctcctcctccgacgtcagcgtcacagcaaacacaggacatctgaccagagaaagactgatttccagcgtcctgcaggggctgcggagacagagcccaaggacaggggcctgctgaggaggtccagcccagctgctgacgtccaggaagaaaacctctagcccacacgatgaagacccccaggcagtgacgtatgccccggtgaaacactccagtcctaggagagaaatggcctctcctccctcctcactgtctggggaattcctggacacaaaggacagacaggtggaagaggacaggcagatggacactgaggctgctgcatctgaagcctcccaggatgtgacctacgcccagctgcacagcttgacccttagacggaaggcaactgagcctcctccatcccaggaaggggaacctccagctgagcccagcatctacgccactctggccatccactagcccggggggtacgcagaccccacactcagcagaaggagactcaggactgctgaaggcacgggagctgcccccagtggacaccagtgaaccccagtcagcctggacccctaacacagaccatgaggagacgctgggaacttgtgggactcacctgactcaaagatg

SEQ ID NO: 8 - DNA, 1398 bp, human
gcgcgcgagccacagcgccggggcgagccagcgagagggccgagcggcggcgctgcctgcagcctgcacgctcggccggccggcgagccagtggccgtgcgcggcggcggcctccgcagcgaccggggagcggactgaccggcgggagggctagcgagccagcggtgtgaggcgcgaggcgaggccgagccgcgagcgacatgggggaccgggagcagctgctgcagcgggcgcggctggccgagcaggcggagcgctacgacgacatggcctccgctatgaaggcggtgacagagctgaatgaacctctctccaatgaagatcgaaatctcctctctgtggcctacaagaatgtggttggtgccaggcgatcttcctggagggtcattagcagcattgagcagaaaaccatggctgatggaaacgaaaagaaattggagaaagttaaagcttaccgggagaagattgagaaggagctggagacagtttgcaatgatgtcctgtctctgcttgacaagttcctgatcaagaactgcaatgatttccagtatgagagcaaggtgttttacctgaaaatgaagggtgattactaccgctacttagcagaggtcgcttctggggagaagaaaaacagtgtggtcgaagcttctgaagctgcctacaaggaagcctttgaaatcagcaaagagcagatgcaacccacgcatcccatccggcttggcctggccctcaacttctccgtgttctactatgagatccagaatgcacctgagcaagcctgcctcttagccaaacaagccttcgatgatgccatagctgagctggacacactaaacgaggattcctataaggactccacgctgatcatgcagttgctgcgagacaacctcaccctctggacgagcgaccagcaggatgaagaagcaggagaaggcaactgaagatccttcagatcccctggcccttccttcacccaccacccccatcatcaccgattcttccttgccacaatcactaaatatctagtgctaaacctatctgtattggcagcacagctactcagatctgcactcctgtctcttgggaagcagtttcagataaatcatgggcattgctggactgatggttgctttgagcccacaggagctccctttttgaattgtgtggagaagtgtgttctgatgaggcattttactatgcctgttgatctatgggaaatctaggcgaaagtaatggggaagattagaaagaattagccaaccaggctacagttgatatttaaaagatccatttaaaacaagctgatagtgtttcgttaagcagtacatcttgtgcatgcaaaaatgaattcacccctcccacctctttcttcaattaatggaaaagcgttaagggaag

SEQ ID NO: 9 - DNA, 1724 bp, human
ctttttggagacagattcgcagtggtcgcttcttctccttggatttgttaaggattccaagtaactcttatttggagagaagacgatctgcacttcgcattttggcattgacatttaattttagggtcctttatatagaagggagagtagctacatgaatgtgtaagatcttggaggaagacagcagagagagagagagagatcagagatcccagggttaaaagttggagaaatttcacagtacatcatccaaaagaggagtccatgatggaggcagaggtaaacttggagaggacaggaagatgtcacccaagcgcatagctaaaagaaggtcccccccagcagatgccatccccaaaagcaagaaggtgaaggtctcacacaggtcccacagcacagaacccggcttggtgctgacactaggccagggcgacgtgggccagctggggctgggtgagaatgtgatggagaggaagaagccggccctggtatccattccggaggatgttgtgcaggctgaggctgggggcatgcacaccgtgtgtctaagcaaaagtggccaggtctattccttcggctgcaatgatgagggtgccctgggaagggacacatcagtggagggctcggagatggtccctgggaaagtggagctgcaagagaaggtggtacaggtgtcagcaggagacagtcacacagcagccctcaccgatgatggccgtgtcttcctctggggctccttccgggacaataacggtgtgattggactgttggagcccatgaagaagagcatggtgcctgtgcaggtgcagctggatgtgcctgtggtaaaggtggcctcaggaaacgaccacttggtgatgctgacagctgatggtgacctctacaccttgggctgcggggaacagggccagctaggccgtgtgcctgagttatttgccaaccgtggtggccggcaaggcctcgaacgactcctggtccccaagtgtgtgatgctgaaatccaggggaagccggggccacgtgagattccaggatgccttttgtggtgcctatttcacctttgccatctcccatgagggccacgtgtacggcttcggcctctccaactaccatcagcttggaactccgggcacagaatcttgcttcataccccagaacctaacatccttcaagaattccaccaagtcctgggtgggcttctctggtggccagcaccatacagtctgcatggattcggaaggaaaagcatacagcctgggccgggctgagtatgggcggctgggccttggagagggtgctgaggagaagagcatacccaccctcatctccaggctgcctgctgtctcctcggtggcttgtggggcctctgtggggtatgctgtgaccaaggatggtcgtgttttcgcctggggcatgggcaccaactaccagctgggcacagggcaggatgaggacgcctggagccctgtggagatgatgggcaaacagctggagaaccgtgtggtcttatctgtgtccagcgggggccagcatacagtcttattagtcaaggacaaagaacagagctgatgaagcctctgagggcctggcttctgtcctgcacaacctccctcacagaacagggaagcagtgacagctgcagatggcagcgggcctctccccagccctgagcactgtgtcagttcctgccttttctcatcagcagaacagaatccttttcctctt

SEQ ID NO: 10 - DNA, 1622 bp, human
cgttggcgtttacgtgtggaagagcggaagagttttgcttttcgtgcgcgccttcgaaaactgcctgccgctgtctgaggagtccacccgaaacctcccctcctccgccggcagccccgcgctgagctcgccgacccaagccagcgtgggcgaggtgggaagtgcgcccgacccgcgcctggagctgcgcccccgagtgcccatggctacaagggtgctgagcatgagcgcccgcctgggacccgtgccccagccgccggcgccgcaggacgagccggtgttcgcgcagctcaagccggtgctgggcgccgcgaatccggcccgcgacgcggcgctcttccccggcgaggagctgaagcacgcgcaccaccgcccgcaggcgcagcccgcgcccgcgcaggccccgcagccggcccagccgcccgccaccggcccgcggctgcctccagaggacctggtccagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactgagcactgcccgtgtgacccgttccaggtcccctgggctccctcaaat

SEQ ID NO: 11 - DNA, 1221 bp, human
cgcaggctggaaggaagacgaacctacgaagcagagatctgaagacagcatgtacacagccattccccagagtggctctccattcccaggctcagtgcaggatccaggcctgcatgtgtggcgggtggagaagctgaagccggtgcctgtggcgcaagagaaccagggcgtcttcttctcgggggactcctacctagtgctgcacaatggcccagaagaggtttcccatctgcacctgtggataggccagcagtcatcccgggatgagcagggggcctgtgccgtgctggctgtgcacctcaacacgctgctgggagagcggcctgtgcagcaccgcgaggtgcagggcaatgagtctgacctcttcatgagctacttcccacggggcctcaagtaccaggaaggtggtgtggagtcagcatttcacaagacctccacaggagccccagctgccatcaagaaactctaccaggtgaaggggaagaagaacatccgtgccaccgagcgggcactgaactgggacagcttcaacactggggactgcttcatcctggacctgggccagaacatcttcgcctggtgtggtggaaagtccaacatcctggaacgcaacaaggcgagggacctggccctggccatccgggacagtgagcgacagggcaaggcccaggtggagattgtcactgatggggaggagcctgctgagatgatccaggtcctgggccccaagcctgctctgaaggagggcaaccctgaggaagacctcacagctgacaaggcaaatgcccaggccgcagctctgtataaggtctctgatgccactggacagatgaacctgaccaaggtggctgactccagcccctttgcccttgaactgctgatatctgatgactgctttgtgctggacaacgggctctgtggcaagatctatatctggaaggggcgaaaagcgaatgagaaggagcggcaggcagccctgcaggtggccgagggcttcatctcgcgcatgcagtacgccccgaacactcaggtggagattctgcctcagggccgtgagagtcccatcttcaagcaatttttcaaggactggaaatgagggtgggcgtcttcctgccccatgctcccctgccccccaccacctgcctgcttgcttctctggctgcctggtcagtgcagaggtgccccctgcagatgttcaataaaggagacaagtgctttccc

SEQ ID NO: 12 - DNA, 1460 bp, human
accccatcttcatctggccttgactctgcccttgaggggcctaggggtgcagccagcctgctccgagctcccctgcagatggaggaggccatcctggtcccctgcgtgctggggctcctgctgctgcccatcctggccatgttgatggcactgtgtgtgcactgccacagactgccaggctcctacgacagcacatcctcagatagtttgtatccaaggggcatccagttcaaacggcctcacacggttgccccctggccacctgcctacccacctgtcacctcctacccacccctgagccagccagacctgctccccatcccaagatccccgcagccccttgggggctcccaccggacgccatcttcccggcgggattctgatggtgccaacagtgtggcgagctacgagaacgagggtgcgtctgggatccgaggtgcccaggctgggtggggagtctggggtccgtcctggactaggctgacccctgtgtcgttacccccagaaccagcctgtgaggatgcagatgaggatgaggacgactatcacaacccaggctacctggtggtgcttcctgacagcaccccggccactagcactgctgccccatcagctcctgcactcagcacccctggcatccgagacagtgccttctccatggagtccattgatgattacgtgaacgttccggagagcggggagagcgcagaagcgtctctggatggcagccgggagtatgtgaatgtgtcccaggaactgcatcctggagcggctaagactgagcctgccgccctgagttcccaggaggcagaggaagtggaggaagagggggctccagattacgagaatctgcaggagctgaactgagggcctgtggaggccgagtctgtcctggaaccaggcttgcctgggacggctgagctgggcagctggaagtggctctggggtcctcacatggcgtcctgcccttgctccagcctgacaacagcctgagaaatccccccgtaacttattatcactttggggttcggcctgtgtcccccgaacgctctgcaccttctgacgcagcctgagaatgacctgccctggccccagccctactctgtgtaatagaataaaggcctgcgtgtgtctgtgttgagcgtgcgtctgtgtgtgcctgtgtgcgagtctgagtcagagatttggagatgtctctgtgtgtttgtgtgtatctgtgggtctccatcctccatgggggctcagccaggtgctgtgacaccccccttctgaatgaagccttctgacctgggctggcactgctgggggtgaggacacattgccccatgagacagtcccagaacacggcagctgctggctgtgacaatggtttcaccatccttagaccaagggatgggacctgatgacctgggaggactcttttagttcttacctcttgtggttctcaataaaacagaacg

SEQ ID NO: 13 - DNA, 1403 bp, human
gcttccgctttggggtggtggtgccacccgccgtggccggcgcccggccggagctgctggtggtggggtcgcggcccgagctggggcgttgggagccgcgcggtgccgtccgcctgaggccggccggcaccgcggcgggcgacggggccctggcgctgcaggagccgggcctgtggctcggggaggtggagctggcggccgaggaggcggcgcaggacggggcggagccgggccgcgtggacacgttctggtacaagttcctgaagcgggagccgggaggagagctctcctgggaaggcaatggacctcatcatgaccgttgctgtacttacaatgaaaacaacttggtggatggtgtgtattgtctcccaataggacactggattgaggccactgggcacaccaatgaaatgaagcacacaacagacttctattttaatattgcaggccaccaagccatgcattattcaaggccgagtacagatgctgccccaggcggtgtgcctgctgcatgcgctgctggagaagggacacatcgtgtacgtgcactgcaacgctggggtgggccgctccaccgcggctgtctgcggctggctccagtatgtgatgggctggaatctgaggaaggtgcagtatttcctcatggccaagaggccggctgtctacattgacgaagaggccttggcccgggcacaagaagattttttccagaaatttgggaaggttcgttcttctgtgtgtagcctgtagctggtcagcctgcttctgccccctcctgatttccctaaggagcctgggatgatgttggtcaaatgacctagaaacaaggattctacctgaactgaaaggactgtgtgacctcccccaagccaaccactttcacctgggatgactttcgattatgctttgttttggggctgtatttttgaaatactctacaagaaagctgtggctcaacacatgagaagaagcacgaagcagttaggctgtacatcagacagaagggtaatgcgtgcagttcctgctgcctgcaggcagacgaggcctttgctttacagcactgtatgtgttgcacgatggatccgtgacagcactttcctgttgcactgaaactcttggccatgtagaggaaaagatatggagttatgtggatttcatcactagtatgtgtgcgtgagctggtcagttgccaaaggaggaaataaggttagaagcctgaaccgttacaaaagaagagctcactatggtcaaaaagtgatggctttcaggacttgttttttatcctgcctcacagttgttaaagtctgttccaaggcatcaccttccttctctacccaacaaccctgtgtaacaactaaagtagaattatctccaaaaaaaaaaaaaaaaaaaaaaaaaaaaaa

SEQ ID NO: 14 - DNA, 3463 bp, human
atggctgagccgactagtgatttcgagactcctatcgggtggcatgcgtctcccgagctgactcccacgttagggcccctgagcgacactgccccgccgcgggacaggtggatgttctgggcaatgctgccgccaccgccaccaccacttacgtcctcgcttcccgcagccgggtcaaagccttcctctgagtcgcagccccccatggaggcccagtctctccccggggctccgccccccttcgacgcccagattcttcccggggcgcaaccccccttcgacgcccagtctccccttgattctcagcctcaacccagcggccagccttggaatttccatgcttccacatcgtggtattggagacagtcttctgataggtttcctcggcatcagaagtccttcaaccctgcagttaaaaattcttattatccacgaaagtatgatgcaaaattcacagacttcagcttacctcccagtagaaaacagaaaaaaaagaaaagaaaggaaccagtttttcactttttttgtgatacctgtgatcgtggttttaaaaatcaagaaaagtatgacaaacacatgtctgaacatacaaaatgccctgaattagattgctcttttactgcacacgagaagattgtccagttccattggagaaatatgcatgctcctggcatgaagaagatcaagttagacactccagaggaaattgcacggtggagggaagaaagaaggaaaaactatccaactctggccaatattgaaaggaagaagaagttaaaacttgaaaaggagaagagaggagcagtattgacaacaacacaatatggcaagatgaaggggatgtccagacattcacaaatggcaaagatcagaagtcctggcaagaatcacaaatggaaaaacgacaattctagacagagagcagtcactggatcaggcagtcacttgtgtgatttgaagctagaaggtccaccggaggcaaatgcagatcctcttggtgttttgataaacagtgattctgagtctgataaggaggagaaaccacaacattctgtgatacccaaggaagtgacaccagccctatgctcactaatgagtagctatggcagtctttcagggtcagagagtgagccagaagaaactcccatcaagactgaagcagacgttttggcagaaaaccaggttcttgatagcagtgctcctaagagtccaagtcaagatgttaaagcaactgttagaaatttttcagaagccaagagtgagaaccgaaagaaaagctttgaaaaaacaaaccctaagaggaaaaaagattatcacaactatcaaacgttattcgaaccaagaacacaccatccatatctcttggaaatgcttctagctccggacattcgacatgaaagaaatgtgattttgcagtgtgttcggtacatcattaaaaaagacttttttggactggatactaattctgcgaaaagtaaagatgtataggcatctggtgtttcagcatacataactgaagcatgtgaaacagtatcatcctcgttagtagaggaaaaccaaaacccttttttccgtcaaaattggatttgtaattaaattgtaagcctcgtaggatgtatgttggaattttaagtctttcctttggttctatgcaaataaaaaaataactgattttttaagactgtgtctgtattgttgggattgaatctagtatttgctgggagaattttttctttgtatttattttaatgtattgttctcatgtaagaatgactgatgttgtgttagttaagaattgaagataggtttagcagtaaagaagaaagcttttaaaaggattgattcagctaagcaaagttgggcagagaaatacagccattttgtttttaatgcagaaaaggaagatgttctgtagcaagggggaatattttaaaaataaaccagatcaaattaatacaatcagaaggtttcgaaatgtaaatattccttatttaagacatgtttaaattcacctactagcacgacttacatagctcaaatattgaatgtttaaaatattaatacagatggggcctctttatgtttagataaaattgaagtacttaattgaagctttttaaaaattgtaaagtaaatgaaagctattgagatctttttgtctcctataataccagggaatttgagcttgtgttctagtcattgtactagctgtagctattggtctgtccttttgacatacagctaaaagggactaaatttgtaaaaaattagtttgttatagttgaagattaacttttcctaacattgtgattattgaagttcatgaatcttgctgtcaaggaagaaaggtaagaaagctgatagctcctccatgttggtaaaatcctctccagaatcttggaacacctggcatgtgaccctagtgacgtcacagacctgagatgaagattcatgtttagccagtgttttccagccttgtacccaccatacagatctgtttattctgtttcaccctactcctccagtgagccccatattttgggaaattatctgccttatacattaactaattcaattcatgtaacactgttgagtgcttactctttgtacctctattgtgcctatattaaaggtatacaaataaataaggccatgtctgacttcaaggaactcagtttaattttgatatattcaaagatgtgattcccaaccaactcaggatgaagtaactagtgttacaactgagttgatattctaaaatataacccagtttgtacttttattactagttagcatacacattttatggcttatgggttaataaatgaattcatggactcctggactactttcattgatgaccatatctccagggatgttgttgatccccacactgccttaaggtatattatagaaacagttttattttccatttttcttgtttcctgataataaatgtatttaggactgaaaatactcctgagtactcccctggctgtatgtctgacagtctttagctatggtgactattgtttatttttaatgggtatttcagattccaagtgtatttaaaatttctaaggagatataatatagcctgtatggtttctactttatggaattatatggtcaatatttgtaaatattctatgagttttgggtgggtagaggggtgctttgcctgttttgggtacaggtttttttggatttagcttgttaattgttcaaactttctgccttctacattcctatcttattgttcgtttaatcagtttctgaaatgtaagcattacatgactattggtgagttgtgccttttataactgaaatactttactttttctcatatcctctataattgacttctattttccttaatcaaaccagctctgggaaatttaatacatttatattaattgagattattaaaacatttggactattaaaaaaaaaaaaaaaaaaa

SEQ ID NO: 15 - DNA, 5115 bp, human
gaattccgggagcgggcgggctgcgaggccgcggggcatgcgggaggcggaggggtgggaccgggtggctgcgcccattccacacccgccgaaagcggacactgtcagctgaatcactccccttttaggaggagggagggggaaaaggtgtctagctaatttctgcttaaaaaagcacaggagatcgcgggtcagctttgcagtcgctgccttctcgcgcctgaccatgcacccctgcatcttcctgctgggcacaggcgagcgctttatttctggagctgagggctaaaacttttttcacttttcttctcctcaacatctgaatcatgccatgtgcccagaggagctggcttgcaaacctttccgtggtggctcagctccttaactttggggcgctttgctatgggagacagcctcagccaggcccggttcgcttcccggacaggaggcaagagcattttatcaagggcctgccagaataccacgtggtgggtccagtccgagtagatgccagtgggcattttttgtcatatggcttgcactatcccatcacgagcagcaggaggaagagagatttggatggctcagaggactgggtgtactacagaatttctcacgaggagaaggacctgttttttaacttgacggtcaatcaaggatttctttccaatagctacatcatggagaagagatatgggaacctctcccatgttaagatgatggcttcctctgcccccctctgccatctcagtggcacggttctacagcagggcaccagagttgggacggcagccctcagtgcctgccatggactgactggatttttccaactaccacatggagactttttcattgaacccgtgaagaagcatccactggttgagggagggtaccacccgcacatcgtttacaggaggcagaaagttccagaaaccaaggagccaacctgtggattaaaggacagtgttaacatctcccagaagcaagagctatggcgggagaagtgggagaggcacaacttgccaagcagaagcctctctcggcgttccatcagcaaggagagatgggtggagacactggtggtggccgacacaaagatgattgaataccatgggagtgagaatgtggagtcctacatcctcaccatcatgaacatggtcactgggttgttccataacccaagcattggcaatgcaattcacattgttgtggttcggctcattctactcgaagaagaagagcaaggactgaaaatagttcaccatgcagaaaagacactgtctagcttctgcaagtggcagaagagtatcaatcccaagagtgacctcaatcctgttcatcacgacgtggctgtccttctcaccagaaaggacatctgtgctggtttcaatcgcccctgcgagaccctgggcctgtctcacctttcaggaatgtgtcagcctcaccgcagttgtaacatcaatgaagattcgggactccctctggctttcacaattgcccatgagctaggacacagcttcggcatccagcatgatgggaaagaaaatgactgtgagcctgtgggcagacatccgtacatcatgtcccgccagctccagtacgatcccactccgctgacatggtccaagtgcagcgaggagtacatcacccgcttcttggaccgaggctgggggttctgtcttgatgacatacctaaaaagaaaggcttgaagtccaaggtcattgcccccggagtgatctatgatgttcaccaccagtgccagctacaatatggacccaatgctaccttctgccaggaagtagaaaacgtctgccagacactgtggtgctccgtgaagggcttttgtcgctctaagctggacgctgctgcagatggaactcaatgtggtgagaagaagtggtgtatggcaggcaagtgcatcacagtggggaagaaaccagagagcattcctggaggctggggccgctggtcaccctggtcccactgttccaggacctgtggggctggagtccagagcgcagagaggctctgcaacaaccccgagccaaagtttggagggaaatattgcactggagaaagaaaacgctatcgcttgtgcaacgtccacccctgtcgctcagaggcaccaacatttcggcagatgcagtgcagtgaatttgacactgttccctacaagaatgaactctaccactggtttcccatttttaacccagcacatccttgtgagctctactgccgacccatagatggccagttttctgagaaaatgctggatgctgtcattgatggtaccccttgctttgaaggcggcaacagcagaaatgtctgtattaatggcatatgtaagatggttggctgtgactatgagatcgattccaatgccaccgaggatcgctgcggtgtgtgcctgggagatggctcttcctgccagactgtgagaaagatgtttaagcagaaggaaggatctggttatgttgacattgggctcattccaaaaggagcaagggacataagagtgatggaaattgagggagctggaaacttcctggccatcaggagtgaagatcctgaaaaatattacctgaatggagggtttattatccagtggaacgggaactataagctggcagggactgtctttcagtatgacaggaaaggagacctggaaaagctgatggccacaggtcccaccaatgagtctgtgtggatccagcttctattccaggtgactaaccctggcatcaagtatgagtacacaatccagaaagatggccttgacaatgatgttgagcagatgtacttctggcagtacggccactggacagagtgcagtgtgacctgcgggacaggtatccgccgccaaactgcccattgcataaagaagggccgcgggatggtgaaagctacattctgtgacccagaaacacagcccaatgggagacagaagaagtgccatgaaaaggcttgtccacccaggtggtgggcaggggagtgggaagcatgctcggcgacatgcgggccccacggggagaagaagcgaaccgtgctgtgcatccagaccatggtctctgacgagcaggctctcccgcccacagactgccagcacctgctgaagcccaagaccctcctttcctgcaacagagacatcctgtgcccctcggactggacagtgggcaactggagtgagtgttctgtttcctgtggtggtggagtgcggattcgcagtgtcacatgtgccaagaaccatgatgaaccttgcgatgtgacaaggaaacccaacagccgagctctgtgtggcctccagcaatgcccttctagccggagagttctgaaaccaaacaaaggcactatttccaatggaaaaaacccaccaacactaaagcccgtccctccacctacatccaggcccagaatgctgaccacacccacagggcctgagtctatgagcacaagcactccagcaatcagcagccctagtcctaccacagcctccaaagaaggagacctgggtgggaaacagtggcaagatagctcaacccaacctgagctgagctctcgctatctcatttccactggaagcacttcccagcccatcctcacttcccaatccttgagcattcagccaagtgaggaaaatgtttccagttcagatactggtcctacctcggagggaggccttgtagctacaacaacaagtggttctggcttgtcatcttcccgcaaccctatcacttggcctgtgactccattttacaataccttgaccaaaggtccagaaatggagattcacagtggctcaggggaagaaagagaacagcctgaggacaaagatgaaagcaatcctgtaatatggaccaagatcagagtacctggaaatgacgctccagtggaaagtacagaaatgccacttgcacctccactaacaccagatctcagcagggagtcctggtggccacccttcagcacagtaatggaaggactgctccccagccaaaggcccactacttccgaaactgggacacccagagttgaggggatggttactgaaaagccagccaacactctgctccctctgggaggagaccaccagccagaaccctcaggaaagacggcaaaccgtaaccacctgaaacttccaaacaacatgaaccaaacaaaaagttctgaaccagtcctgactgaggaggatgcaacaagtctgattactgagggctttttgctaaatgcctccaattacaagcagctcacaaacggccacggctctgcacactggatcgtcggaaactggagcgagtgctccaccacatgtggcctgggggcctactggaaaagggtggagtgcaccacccagatggattctgactgtgcggccatccagagacctgaccctgcaaaaagatgccacctccgtccctgtgctggctggaaagtgggaaactggagcaagtgctccagaaactgcagtgggggcttcaagatacgcgagattcagtgcgtggacagccgggaccaccggaacctgaggccatttcactgccagttcctggccggcattcctcccccattgagcatgagctgtaacccggagccctgtgaggcgtggcaggtggagccttggagccagtgctccaggtcctgtggaggtggagttcaggagagaggagtgttctgtccaggaggcctctgtgattggacaaaaagacccacatccaccatgtcttgcaatgagcacctgtgctgtcactgggccactgggaactgggacctgtgttccacttcctgtggaggtggctttcagaagaggattgtccaatgtgtgccctcagagggcaataaaactgaagaccaagaccaatgtctatgtgatcacaaacccagacctccagaattcaaaaaatgcaaccagcaggcctgcaagaaaagtgccgatttactttgcactaaggacaaactgtcagccagtttctgccagacactgaaagccatgaagaaatgttctgtgcccaccgtgagggctgagtgctgcttctcgtgtccccagacacacatcacacacacccaaaggcaaagaaggcaacggttgctccaaaagtcaaaagaactctaagcccaaa

SEQ ID NO: 16 - DNA, 528 bp, human
cgccagggagctgtgaggcagtgctgtgtggttcctgccgtccggactctttttcctctactgagattcatctgtgtgaaatatgagttggcgaggaagatcgacctattattggcctagaccaaggcgctatgtacagcctcctgaaatgattgggcctatgcggcccgagcagttcagtgatgaagtggaaccagcaacacctgaagaaggggaaccagcaactcaacgtcaggatcctgcagctgctcaggagggagaggatgagggagcatctgcaggtcaagggccgaagcctgaagctgatagccaggaacagggtcacccacagactgggtgtgagtgtgaagatggtcctgatgggcaggagatggacccgccaaatccagaggaggtgaaaacgcctgaagaaggtgaaaagcaatcacagtgttaaaagaaggcacgttgaaatgatgcaggctgctcctatgttggaaatttgttcattaaaattctcccaataaagctttacagccttctgcaaaa

SEQ ID NO: 17 - DNA, 2247 bp, human
tttcttgagctaggaaaggtggttggcttacggcacagtagagagcttccagggctggctggcgtgggatacccgtaccacagaaatgcagggaccattgcttcttccaggcctctgctttctgctgagcctctttggagctgtgactcagaaaaccaaaacttcctgtgctaagtgccccccaaatgcttcctgtgtcaataacactcactgcacctgcaaccatggatatacttctggatctgggcagaaactattcacattccccttggagacatgtaacgacattaatgaatgtacaccaccctatagtgtatattgtggatttaacgctgtgtgttacaatgtcgaaggaagtttctactgtcaatgtgtcccaggatatagactgcattctgggaatgaacaattcagtaattccaatgagaacacctgtcaggacaccacctcctcaaagacaaccgagggcaggaaagagctgcaaaagattgtggacaaatttgagtcacttctcaccaatcagactttatggagaacagaagggagacaagaaatctcatccacagctaccactattctccgggatgtggaatcgaaagttctagaaactgccttgaaagatccagaacaaaaagtcctgaaaatccaaaacgatagtgtagctattgaaactcaagcgattacagacaattgctctgaagaaagaaagacattcaacttgaacgtccaaatgaactcaatggacatccgttgcagtgacatcatccagggagacacacaaggtcccagtgccattgcctttatctcatattcttctcttggaaacatcataaatgcaactttttttgaagagatggataagaaagatcaagtgtatctgaactctcaggttgtgagtgctgctattggacccaaaaggaacgtgtctctctccaagtctgtgacgctgactttccagcacgtgaagatgacccccagtaccaaaaaggtcttctgtgtctactggaagagcacagggcagggcagccagtggtccagggatggctgcttcctgatacacgtgaacaagagtcacaccatgtgtaattgcagtcacctgtccagcttcgctgtcctgatggccctgaccagccaggaggaggatcccgtgctgactgtcatcacctacgtggggctgagcgtctctctgctgtgcctcctcctggcggccctcacttttctcctgtgtaaagccatccagaacaccagcacctcactgcatctgcagctctcgctctgcctcttcctggcccacctcctcttcctcgtggggattgatcgaactgaacccaaggtgctgtgctccatcatcgccggtgctttgcactatctctacctggccgccttcacctggatgctgctggagggtgtgcacctcttcctcactgcacggaacctgacagtggtcaactactcaagcatcaatagactcatgaagtggatcatgttcccagtcggctatggcgttcccgctgtgactgtggccatttctgcagcctcctggcctcacctttatggaactgctgatcgatgctggctccacctggaccagggattcatgtggagtttccttggcccagtctgtgccattttctctgcgaatttagtattgtttatcttggtcttttggattttgaaaagaaaactttcctccctcaatagtgaagtgtcaaccatccagaacacaaggatgctggctttcaaagcaacagctcagctcttcatcctgggctgcacatggtgtctgggcttgctacaggtgggtccagctgcccaggtcatggcctacctcttcaccatcatcaacagcctccaaggcttcttcatcttcttggtctactgcctcctcagccagcaggtccagaaacaatatcaaaagtggtttagagagatcgtaaaatcaaaatctgagtctgagacatacacactttccagcaagatgggtcctgactcaaaacccagtgagggggatgtttttccaggacaagtgaagagaaaatattaaaactagaatattcaactccatatggaaaatcatatccatggatctctttggcattatgaagaatgaagctaaggaaaagggaattcattaaacatatcatccttggagaggaagtaatcaacctttacttcccaagctgtttgttctccacaataggctctcaacaaatgtgtggtaaattgcatttctcttcaaaaaaaaaa

SEQ ID NO: 18 - DNA, 1325 bp, human
accaatcctcacctctcacctctgtgtccgccctgctgggaaatattccaggctttggccaaggccagtgcagccccaggttcccgagcggcaggttgggtgcggaccatggcctctcacaagctgctggtgaccccccccaaggccctgctcaagcccctctccatccccaaccagctcctgctggggcctggtccttccaacctgcctcctcgcatcatggcagccggggggctgcagatgatcgggtccatgagcaaggatatgtaccagatcatggacgagatcaaggaaggcatccagtacgtgttccagaccaggaacccactcacactggtcatctctggctcgggacactgtgccctggaggccgccctggtcaatgtgctggagcctggggactccttcctggttggggccaatggcatttgggggcagcgagccgtggacatcggggagcgcataggagcccgagtgcacccgatgaccaaggaccctggaggccactacacactgcaggaggtggaggagggcctggcccagcacaagccagtgctgctgttcttaacccacggggagtcgtccaccggcgtgctgcagccccttgatggcttcggggaactctgccacaggtacaagtgcctgctcctggtggattcggtggcattcctgggcgggacccccctttacatggaccggcaaggcatcgacatcctgtactcgggctcccagaaggccctgaacgcccctccagggacctcgctcatctccttcagtgacaaggccaaaaagaagatgtactcccgcaagacgaagcccttctccttctacctggacatcaagtggctggccaacttctggggctgtgacgaccagcccaggatgtaccatcacacaatccccgtcatcagcctgtacagcctgagagagagcctggccctcattgcggaacagggcctggagaacagctggcgccagcaccgcgaggccgcggcgtatctgcatgggcgcctgcaggcactggggctgcagctcttcgtgaaggacccggcgctccggcttcccacagtcaccactgtggctgtacccgctggctatgactggagagacatcgtcagctacgtcatagaccacttcgacattgagatcatgggtggccttgggccctccacggggaaggtgctgcggatcggcctgctgggctgcaatgccacccgcgagaatgtggaccgcgtgacggaggccctgagggcggccctgcagcactgccccaagaagaagctgtgacctgcccactggcacacagctggcactggcacacacct

SEQ ID NO: 19 - DNA, 2263 bp, human
agccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacagccctccagccaagaagaaatccacaggttcctccacatggcccctggaccctggggtagaggtgaccctgacgatgaaagcggccagtggtagcacaggcgaccagaaggttcagatttcatactacggacccaagactccaccagtcaaagctctactctacctcaccgcggtggaaatctccctgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgagtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtac

SEQ ID NO: 20 - DNA, 2772 bp, human
cagtcggcaccggcgaggccgtgctggaacccgggcctcagccgcagccgcagcggggccgacatgacgacagctccccaggagccccccgcccggcccctccaggcgggcagtggagctggcccggcgcctgggcgcgccatgcgcagcaccacgctcctggccctgctggcgctggtcttgctttacttggtgtctggtgccctggtgttccgggccctggagcagccccacgagcagcaggcccagagggagctgggggaggtccgagagaagttcctgagggcccatccgtgtgtgagcgaccaggagctgggcctcctcatcaaggaggtggctgatgccctgggagggggtgcggacccagaaaccaactcgaccagcaacagcagccactcagcctgggacctgggcagcgccttctttttctcagggaccatcatcaccaccatcggctatggcaatgtggccctgcgcacagatgccgggcgcctcttctgcatcttctatgcgctggtggggattccgctgtttgggatcctactggcaggggtcggggaccggctgggctcctccctgcgccatggcatcggtcacattgaagccatcttcttgaagtggcacgtgccaccggagctagtaagagtgctgtcggcgatgcttttcctgctgatcggctgcctgctctttgtcctcacgcccacgttcgtgttctgctatatggaggactggagcaagctggaggccatctactttgtcatagtgacgcttaccaccgtgggctttggcgactatgtggccggcgcggaccccaggcaggactccccggcctatcagccgctggtgtggttctggatcctgctcggcctggcttacttcgcctcagtgctcaccaccatcgggaactggctgcgagtagtgtcccgccgcactcgggcagagatgggcggcctcacggctcaggctgccagctggactggcacagtgacagcgcgcgtgacccagcgagccgggcccgccgccccgccgccggagaaggagcagccactgctgcctccaccgccctgtccagcgcagccgctgggcaggccccgatccccttcgccccccgagaaggctcagctgccttccccgcccacggcctcggccctggattatcccagcgagaacctggccttcatcgacgagtcctcggatacgcagagcgagcgcggctgcccgctgccccgcgcgccgagaggtcgccgccgcccaaatccccccaggaagcccgtgcggccccgcggccccgggcgtccccgagacaaaggcgtgccggtgtaggggcaggatccctggccgggcctctcaagggcttcgtttctgctctccccggcatgcctggcttgtttgaccaaagagccctctttccacgagactgaagtctggggaggaggctacagttgcctctccgcctcctccctggccccggcccttccctcacttccatccatctctagacccccccaaggctttctgtgtcgctgccccgggcgggtgtatccctcacagcacctcacgactgtgcctcaaagcctgcatcaataaatgaaaacggtctgcaccgctgcgggcgtgacgctcccggacgcgagtgggtgtggaattgctttcctcgggccaccgtgggggcacctctggcctcccgtgacccccaggccgagggtccccgggcacccaggtcggtcaagtctcggccctctcaggcccgcgtctctgcctggaggagactgtgtagggtccggcgtggggatcagccgggatgggctgcgcgtctccagcctctgcacacacattggcgggtggggtgcagggagggagaggcaggggagagagaatggcatctcgcgtggagggctgtcgtttgaactctcccagcgcgagagaccctgccccgcccccttcctggagcgttgactcccttctcgtctcgaggcctgtggcgtctgggtccgttggggcagaaccatggaggaaaagccttcgaaagtgtcgctcaagtcttccgaccgccaaggctcggacgaggagagcgtgcatagcgacactcgggacctgtggaccacgaccacgctgtcccaggcacagctgaacatgccgctgtccgaggtctgcgagggcttcgacgaggagggccgcaacattagcaagacccgcgggtggcacagcccggggcggggctcgttggacgaggggtacaaggccagccacaagccggaggaactggacgagcacgcgctggtggagctggagttgcaccgcggcagctccatggaaatcaatctgggggagaaggacactgcatcccagatcgaggccgaaaagtcttcctcaatgtcatcactcaatattgcgaagcacatgccccatcgagcctactgggcagagcagcagagcaggctgccactgcccctgatggaactcatggagaatgaagctctggaaatcctcaccaaagccctccggagctaccagttagggatcggcagggaccacttcctgactaaggagctgcagcgatacatcgaagggctcaagaagcgccggagcaagaggctgtacgtgaattaaaaacgccaccttgggctcgagcagcgacccgaaccagccccgtgccagcccggtccccagacccaagcctgaccccatccgagtggaatttgagtcctaaagaaataaaagagtcgatgcatgaaaaaaaaaaaaaaaaaaaaaaaaaaaaaaaaaaaaaaa

SEQ ID NO: 21 - DNA, 7883 bp, human
ttcaagtatggcagacaaaggatgttctgcgtggggaaatgtggtgacacccatttcacaaggacagctcacatagattgagtgctcaggaaggaccagcaccatacccagtgcctgatgtgtatcatctcaattagtccttgcctcagatgcaaaaggaaaccatcgccatcatcatcaccaccatcatcatcttcctcctgtgcagatggaaaggctgaggcatagagaggtgacggagtctgcccaggactgcaagcctgctggtggcagagccaggttccaatggaatgaaggctgtcatcctcagatggcagggtaggcaggtggctagagctcacttgggagaaggggaaaggacactgactttggctagggatggagcagagcttgggctggctttccatgcacgggcagggggcgtggctcatggctacgctccagccccgggtgtggacatttaatcttccaggtctaccctaggctatgggtctggacagcactgtgatggaaagaagacactctatgtcctgcattctgtgaccaatgatgtgactgtgggaatggcgctggcatctggctgccactctgggacgggtggccagctgccatcaggccccacccaggatgggaccaccatgcgacttcttccctcgctcctcctggtcatgtccagagccccaggaggaccagcaaagcctctcgagccgatggcagctcacgttctgccttgtcagctactcctctcctgggcaatattggctgcttgctgtggctctccccggggtatgtgactgcctctgtgctgggcacctggcctgggctttccttctgggcctgggcagctgggctcagcttggacccaggcagcagccacagaggggcccatggaggtgacagagttgcttctatgatggtgaacgggcagctgtgacacggaggaggcgaccactcctgagtttccaagtgctgcggtcagggccggggccagcaaagtccctcccatattcaaagagcgggtttgggtttgtcccaggaggacatagtcaggagcccatgctgggacatgcctcctccaaagttcagcctggatccccagcctctgccaacggccccgctccttagctaacccagcttgctcctgggttccacggcggagtcagatgtttctgggcagtttcacctttgtgccttaaatgcatgttgaggactttaaggaattgtggagaaatagggctgtggcaaaggcaagtgacaactgggaacaatgatcccgcagaggctgctgaggcctgggccccaggggcgtgggttcatccttctgcctgggctttggtgggaggggcagactctgtggtctgagacacaaaaaaacccaaaacatatgtgtgtacagacacacagcagagccacacacacacttgtgcccatgcacacactcacaggaggcccgtggactccgcacagggaagaaactcctccggtcgacagtggacggcgctgcagcagggactcacccccaagccctgcctgcctcccattgcccacctggccctggcttgatgggcttatctcatgctgtggccggggacctcttgcttcctgcaaccccttgctggactggggcctgggcctctcctgggctgtgcctagggtttgtaacccagggcctgtgccggcgtgcacagagcatctctccctgggaggctcagggctgcctcctcgagctctgtgggcctgcactggccggtgagcttgtggtgtgggttttcaggctgtatccttctacctcctgagcccaggggtcccaggcgccctgcagctgtctcctcggccatcctgtggggccccgaggccttgccctcacttcagtgcctgggtgctcaggctttgcccaggtgccaggagaaggtgtgagcatgagcctattggacacacctggcgacgtataccaggtgtcccacccctgccaccatggggcctcccgatacggcaaccaccacggacctgtggggaccaatgaggaaagagagaggcaggtctgggccaggctcacagggactccggcatagcagaccctgccccagcaggcccccttgtccttcctgggtcctggtccttcatgaggaactagcccatccctggtggggctcccaccccgcttctcagtgggctctatgcttgcctcgtcggagtcacccctcaggcagtcctgggatcctctcctttagacccactgtgccttcccggcctcccgggcttctgctgggggcagaagaaatgcctccccaggtctgtctctggaggctctgagggagatgggcttgggggctgtaggaggaggcagggattccagggtgtcaggaaggcaggggtgccaggtcccacctagtgaagtaataaaccgtgggtggtgatagtgacccagtgccctcactgcccagccccgcctgtcctcagccagcactgcagggatcccaggcccagactctggaggccttcactgatcccagccaccccagaaaagctgcagcctgcaggcaccagccgggccatatgcccagtgccagctagggcccaccgcccatcctgcacacggggccgctgggcaggtgcccctcacacccccaggatgtcagtgctcacctcgagcaaagcgccccagctcggccttgggaggtggtcatgtccagggggatgatggagagctgtccaaccaagagagcgggagggagggaaggagggagggagagagatagagagagagagagagagagaggaagtgtgggccctaaggctgccttagtggaggtgcgcgtggcctgcacctcaccaagcctagccactctcgcggctctgagtggctcacaggcttgtgagggccccgtcgctgcctgctgggtccccaccagggctccctctaggaatgcgccatggctgctatgacaatttgcacagcccagtggcttaaacaccatttataccacaggtccagatgaatcctgcagggccagggtctgggggtgctggaggccatgctccctccaggcttgcggggagaacttccctgcctcctccagtctctccatccctgagctctcggctcctcctccgtcttcagggccagggcgtagcgtctgctctctcggcctctgcctccgcttcccacctcacctggcttctgtctatgtcagtctccctctgccaacctcctagaaggacacttgtgattacattagggctcacccctttaatccaggggagcctctccacttcatgattttcagctaacttgcttctgcacagaccccctttccctataagggcacacattcactggtcccggggctaaggaccttgctccaagtccctccacccatgatgctgtgccttccagaaacctgtcctctgcagctcggtcttgaccccaagcctgctggtgacctgaacttcacagggttatccccttggactgtgtgcagcacgatgcaatttctgggcctgaatgtcatgctccctggggcaggaccttgagcctgcagcacacactaggccacctgcagtctcacaggccatgccctgggtagacagggaggtgctcaaccccagctcgggtcctctagtctgcctggctaccatgcttctcactctcctgcatctgcagaccctgcgttgccatgtgaggcaggggtggggtggggctgagggcgtggctttggtccctggctgtccggatgaagtaccagagtgacgccacagcccatcccggtgacatgctcacccccaacccccgtgtccgggaccccggtcttgtgtggtccctgatgtggagtcctcagtccttaagatacatccagaaagtcctggccatgaattggaggtgcagagtcctgcagagcctctgggctgggctggtgcccccaggagatggagggcctggtggatgccctcctccctcagagctggggcagctgcctcccaggggtgggactctgggctcagagagaggcccttgagctgcagctcagggggatgcgaggcttcgtggactgtgtcctggtccatgtggtgcacgtgtctccacctccaaggagaggctcctcagtgtgcacctcccccacatccgtcctctctgccggccccgggcgtctgagcagtcattccatgccagcacctctgcagcctgctgggcctcaggttctctgtgagggacctccccggccttcggcggaggtggagtaagctccgtcaaggcaggtggcttcgtcccttcctgtgagtgacaccagtgatgaaatggacccctccacacaggcatcctcagggcacagggccctgggggcaccttcctcctttcgtatttgttgagaaaaaaagtggcattgcgctcacaccaggatgctggagcagagctgacatgctcgggaaagggcagaggtcactgggggtgggaaggtcatccagtccagactcagcacctcgtgggctggtaaactgaggctcaaagtgctggtgccaggcctgaggcctcgcggtgacccctctctctggttcccagcacctgcctgagacctgccccaggcacccataacctggaattccctgtttccttgtccagggcctgaggaaatggctccccaggtctgtctctggatgctctgaggcagatgggcttgggggctctaggaagaggcagggactccagggtgtcaggaaggcaggggtgccgggtcccacccagtggagtaacaaactgtgggtggcgtttgggcctccccgccttccccactgggtgtgctggtgctggcgctgctgggtcagggctgcccgtgaccccagacaccactgtccatcctgtgaggctcccgtctgggcatgtcctgggtggattcctcctttctgttaagtagctacatgaggcaggggctcctggatccaaagcaaatgacaggaattccagagccaggtgcatccactcagggcagccagtgttggtggagctgcctctagcacatggaggagagtgaaagtcagcctgcccctctcacgagaaaagaacctggggatacctctcagcctccagcgttgcaagtgcaaggccagtggagttaatctgcaacgtgcacgagggcgtgtgtcagtggctgtgtgcaggagtgtgagtgagcaagagcaagagcgcatggctcctgctgtacctcaaggtgtgggctcctggtggctgctcagtgttcccaggggtgagaggcctcatgtatcctaggctgcctgagatttctgtgtgctgatcgcatcctcagtttcttgtccaccgcttcactggcaagagtcccaggctccaaggacaccctccctgcacatgattgggtgttaatggtggcctgggttgtgtcttcccctggggatgagggttgggtgtccatggtgccctgggctgtgtcctcccctagggatgagggtcgggcctccacgatgccctgggctgtgtgctcttatgggaatgagggttgggtgtccaagatgccctgggctgtgtccttccctggggatgagggttggatgtccaagatgccctgggctgtgtactcccctaggaatgagggctgggtgtccaagataccctgggctgtgtcctcccctggggatgagggttgggtgtccatggtgccctgggctgtgtcctcccctggggatgacggttgggtgtccatggtgccctgggctgtgtttccttggggatgagggttgggtgctatggcatcctgggcaggtgcttcctttctgcacaagggttgggtgaccatgatgtcctggcaatggcttccctgggttgcctcttttctgccatgtgggaagagcaggggaggtttagttggtctcagcacatcattctctcaggataagtagaagagtgtctgagctgtgaggccagtgctccagctttggaattgtcttccccaccctcacctccatcccatcaaagcccgacatgtcgtgtggcagcagcgaggtgggtgttggctgttctcttgggctgggggttagtcgtggacggggaaaggagagatgctggtcaaagggcatgaagtttctgctgatgggaggagtcagttcttttgatctgttgcacagcatggtgactatagttaacaataatgactatttcaaaattgctaaaagatgagattttaaatgttctcaccacaaaatgataagtgtgtgaggtgatggatatgccacttaccttgttttaatcatcccacaatatagacaggcattgtcactttgcattgtaccccaggaatcttcacatttgcttttttgtcaattaaaaatagagacacaaaaggagagaggggagagcaatagactcttcacggaaccgtgggcttctgcctccgggtaaaataaactgcaaaaaggattcccaggaaaccgttccctctttcagcccttggttacaggaagccggatttgggaaatctgcctggatgacattcacatgaacgggcacatacaggaaaacacggtaatgtaattagaatagtcagagaaaagtagccagaaatgacattcacatgaacgggcacatacaggagaaaacacggtaacgtaattagaatagtcagagaaaagtagccagaaatgacattcacatgaacgggcacatataggagaaaccatggtaacgtaattagaatagtcagagaaaagtagccagaaatgacattcacatgaacgggcacatacaggaaaacacggtaatgtaattagaatagtcagagaaaagtagccagaaatgacattcacatgaacgggcacatacaggagaaaacacggtaacgtaattagaatagtcagagaaaagtagccagaaatgacattcacatgaacgggcacatacaggagaaaacacggtaacgtaattagaatagtcagagaaaagtagccagaagaatttgcaacgtgcccttgtaacaccaaatttgatcagttttttaaaaaatgatcgttatgtaggtgattgagaagtaaatgtattcttttttaaggtaaaaatttggacccttatcatgcatacccccctctgtgctcttcaaatcaacatcattattaatatctgtacatttttgctcatctgagccagcacaggctgaggctgtcagaatggacaccttttggttgttgggtttctgtcagtttctggggtgaagctgcgtgattgagaacgtagctcttggctgccatctcggggattattaaggactgtgaactctatccacaagccatggcaatatctgtcccaccgaatgctccctctaacacactcttactcccgtgatgtgtgttaagggctccgatgatgctgaaaacagcacaggatgtgaaaaggcaggaacagttctgaagtcaaaggctgatgtcctgtttctctttccctctgtgaccgactcccttcccagtggtaacaagtacccacagcttggtttgaatttctgcacgctgttgtctgtgcactcgctcacacttacgcacacagcaggcatgtgggcgatgctgggtattttgtgtatgagtgggatgcacatacacacatctacatccatatcatgcccatgcatctgtaacttgcttttcccgtgtaagaacacttcttagagtttgttcaatgcatgtgtctgtgtgaatgattgaaggcatttctaacccattttaaagatggctacttaggaccatatggatgttgtactgatgtcatttgaccacgtccattgtttccatcttttgggctgttcttgtgtattttactttccatgtaacactgtgacattgagaattggtacctacaacagtctatttgctttacattaaatttgtaggct

SEQ ID NO: 22 - DNA, 1072 bp, human
agtcagtgaaacggcagaatcagaagaggttccacaaccagaaaatcttggctggaatttcaccatcaggaataaaacagaaaaactaaaagagtgccccagatagcctttcttaggggcctgtgacaggtcgcaggaatcttgttggtgatccatccagatgttgtgtgttctggaagtggacatcgcggctctgtgtttttgaagtcagatctcattgctgtggtttctatgcctgaccccccgaagttcttgctcctgttgccacagggagccgggagagcacagagcgctgctcccggtgccctgcagccacacaaacatgctcctgctcctggcggaggcagagctgctgggaaagacatttcggaagtttcctgtggctgcaacaaattgttcaaatctgcactggagcaccgctgtgacctgtctttctccatcttagggcaaacagctcctgaaactggaaactccccagcacctactcaccctacccctcaggctctccttgtgggggtggggcagggggagttgtctggaatgcctggcctctctgtccaagcatggcagccttgccccatgggtggtgcagactcagtttcccatgcaccttgccccagggaggaggtaggggttccttccatagagatggtgaagaataagggaggtagtgatcgtctctgggatccagttagatctgcgtttgcaggcagaaagaggctggggcacatggagagagtgatcaactggaagattctagggtcctcaattttgaaaggtgacatgataccctggaaagggcatgaacttagttgtcagttcgtccttgccttttccaatcaatgctgtgtggccacggcaaattaatgaacatctctgagtttcggtctcctgtctaaaatgaggtgataatagcttcttgaaggttgtaaggccccaaacatgctgcctggcacatagatggctaatcaatattttcctacccttcccttccttcccttctctggagttgctacctgtcttctcctggggccttgcaaataaacttctgaattaaaaaaaaaaaaaaaaa

SEQ ID NO: 23 - DNA, 417 bp, human
acctcccaaccaagccctccagcaaggattcaggagtgcccctcgggcctcgccatgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatgaggacctgaagggtgacatccaggaggggcctctgaaatttcccacaccccagcgcctgtgctgaggactcccgccatgtggccccaggtgccaccaataaaaatcctaccg

SEQ ID NO: 24 - DNA, 1004 bp, human
ttcctcattaaagtttcacaaataaagcacagcaagacttgtctgcagacacacaggaggcacacggacagcccgtcaaccagagatggagacgaaggccagcatggctctcacagggcagcgcttctcagaacccctggcccccctcgtgccaaggctggcctgtgtcaggcctcgcccacgccgccttatgacaaatagagccggtgccaaggaggtggctacagagcaggggcaaggaagttatcctcatgttctgataatgaccctgcaaatcccaccccaccctcaggcacctccgtctaaggtgtccggttactccaggtaaggaggttcccaggagggccgtgttttccctagggctgatgaaacttgctccgacaagccaggccactgggaggcacctcaggatggaaaagatgctgagaggctttgctggctttcaggatgccggggccccacgggggcaaaaggggaggaaggaaagaattctaaagacagattgctgctggtctgtcccgacccagggtcacagtgtcagcaaagagaacagcatgattctgacagggttggattttgtttcaccctcggaatgagcagacattcaaacacttgcattttcacggaaatcaacaagagagacagctagcaggacacgaggctcctgccagttctgtgtggaaaggcaccagatggtttgttatgaaacacattttggtcagaaaatagctggggttttttggttcctgggaggacaacaaagctagaagaaaagaggtgtgagttgcgtgaggaggaggcagagaagaaagcagctttggcatcagacctgggttctactcttcactctacccctcacgcttgaggcctcagtttcctcatctgtaaagtggtcatagaatatttccaaataaatctaggtgtcaggtttcacacattcccaggaagtatggggaggcggggcgcagacactcaaacggacacacagaaaccagaggaagagc

SEQ ID NO: 25 - DNA, 2123 bp, human
tagctgatcatgtgacaatccaagatggcggtgcccggcgaggcggaggaggaggcgacagtttacctggtagtgagcggtatcccctccgtgttgcgctcggcccatttacggagctattttagccagttccgagaagagcgcggcggtggcttcctctgtttccactaccggcatcggcctgagcgggcccctccgcaggccgctcctaactctgccctaattcctaccgacccagccgctgagggccagcttctctctcagacttcggccaccgatgtccggcctctctccactcgagactctactccaatccagacccgcacctgctgctgcgtcatctcggtaagggggttggctcaagctcagaggcttattcgcatgtactcgggccgccggtggctggattctcacgggacttggctaccgggtcgctgtctcatccgcagacttcggctacctacggaggcatcaggtctgggcccctttcccttcaagacccggaaggaactgcagagttggaaggcagagaatgaagccttcaccctggctgacctgaagcaactgccggagctgaacccaccagtgctgatgcccagagggaatgtggggactcccctgcgggtctttttggagttgatccgggcctgccgcctaccccctcggatcatcacccagctgcagctccagttccccaagacaggttcctcccggcgctacggcaatgtgccttttgagtatgaggactcagagactgtggagcaggaagagcttgtgtgtacagcagagggtgaagaaataccccaaggaacctacctggcagatataccagccagcccctgtggagagcctgaggaagaagtggggaaggaagaggaagaagagtctcactcagatgaggacgatgaccggggtgaggaatgggaacggcatgaagcgctgcatgaggacgtgaccgggcaggagcggaccactgagcagctctttgaggaggagattgagctcaagtgggagaagggtggctctggcctggtgttttatactgatgcccagttctggcaggaggaagaaggagattttgatgaacagacagccgatgactgggatgtggacatgagtgtgtactatgacagagatggtggagacaaggatgcccgagactctgtccaaatgcgtctagaacagagactccgagatggacaggaagatggctctgtgatcgaacgccaggtgggcacctttgagcgccacaccaagggcattgggcggaaggtgatggagcggcagggctgggctgagggccagggcctgggctgcaggtgctcaggggtgcctgaggccctggatagtgatggccaacaccccagatgcaagcgtggattggggtaccatggagagaagctacagccatttgggcaactgaagaggccccgtagaaatggcttggggctcatctccaccatctatgatgagcctctaccccaagaccagacggagtcactgctccgccgccagccacccaccagcatgaagtttcggacagacatggcctttgtgaggggttccagttgtgcttcagacagcccctcattgcctgactgaccgggttgggggcttcctttcatagctacatgatgaaaaccctctgccctggcctcatctaccactgaagcagaaaggagtctgggagcagcagtcttcgtggctggttcagggtgttttgttccgagcctgcctgcctgccggttctatacctcaggggcatttttacaaaaagccccctcccgtcccctccccttggatattaggggtaacgaccgcttgtctttggtctctaaccctaatctctgggcttgccctttgcctcctgcagaactttgaaaagctgggttgagtgaggctatcagcacagccttccttggggactctgaaggtgtccccacgaaggccagaaagggggaaagggacctgggcgaggagaggatttgtggtgcttggaagagccggccttgggtgggccctccaccgcctctaccctcactgggtgggactgccagcggagagtccgcgggaggtggcttgggtgtgcgacgtcacggaagaataaagacgtttactactgg

SEQ ID NO: 26 - DNA, 1276 bp, human
ggaatccacccggggtgtgtggattcctgccctgttcccacaggacagccctcaaccaatggagacaggaacctggagttaaatgcttctccctttttcactgagagagagacatgcacagtctgatgcactttctttccttctttctttttctttctttttttttcttaagacagagtctctctctgtcaccaaggctggagtgcaggggcacgatctgggctcactgccacctccacctcccgggttcaagcaattctcccacctcagcctcccgagtagctgggattacaggcactagttaccacgcccagctaatttttgtatttttagtagagatgcggtttcaccatattggtcaggctggtctcagactcctgatctcaggtaatctgtctgcctcagcctcccaaggtgctggaattacaggcatgagccaccacacctggccgtgatgcactttctagatgctgtcctagagatcacactgtgttaagcctcagttgccttcaatgtggtcatctctacagtatacccttagcttttttctcctccgttactttcccagaccctcactctgctccctggattcacttttcgaaatagtcctcctgctgcaaagtcctgggcacctgccctactttcagcattggaaggggggcccaggctaagaccatgaggccccactgtgggcgcccacagccccgttcctccctctattcccaccacagtcacatcctcctgtccctcagtgcttcctcgcctttccctccagcccaccgtgagatcccaggggacggagcagccccttctctgccccagtgcagggcttggccttagcacacggtcagtctgtgctggggtgaagtgatgaatgagtgagtggttgagtgataatgcatcatcagatctgtcttttccacatgtctctatctccacccagaaccagttttctcatccacaaatgggcatttgaggctgggtgctcctaaaccctacaaaattcagagctggcacagttggggactgaccttccttgatctcacctcactttctgtatctataaaatggggtacctttctctaagagtaaaaaggaggcctggcatagggaaagaaactcagctcgagcatccagaacatccatcttgctctcaaatacctaatacaggggaccatgttttctgctataattggtattggagctggtaccatttattaaaggtaattcagttacaaagcttcaaaaaaaaaaaaaaaaaaaaaaaaaaaaaaaa

SEQ ID NO: 27 - DNA, 7764 bp, human
ccctgggatggaggatctgtctctctctctctctctcctttttttttttttggtggagatgaaggggtgggtctatggtacatcacctgagttgtggggtaaatgtagagagtgtcaatcaaaggcagagctctcagagctgggaaggaggctctagatggcggctgtgccttagagagagcgcgctctgctccctgcctttgcctcactttacgcaactttccctaactttcgggcagcctcagggggcccccgtagccccctgcctttcctagggacttactggggtcgattcgaacctttttttgggagaaaagcagcttttaggagctttcttttcgtgccttgttggaaagaagcagccgtactgagagcccaggtcgttgttttttccagcttagaagccatggcgcacctccatttttgtgcgctctcctaatgaggttttttttctttcggacctgttttagtattaattattgctttatttttttgaccagttaacatatttgagggttattttatttatttttcgttttttaacggaggattttgcctttatttttaattatttgggatctgatatttttctactagtagataggactcttggtttggacatactacatggatcagtaaatacctgggcacaggacttcaaagcaaacacagattccccctcccccttaatatttaagaattaaaagatgatgagaaataaggacaaaagccaagaggaggacagttcgctacacagcaatgcatcgagtcactcagcctctgaagaagcttcgggttcagactcaggcagtcagtcggaaagtgagcagggaagtgatccaggaagtggacatggcagcgagtcgaacagcagctctgaatcttctgagagtcagtcggaatctgagagcgaatcagcaggttccaaatcccagccagtcctcccagaagccaaagagaagccagcctctaagaaggaacggatagctgatgtgaagaagatgtgggaagaatatcctgatgtttatggggtcaggcggtcaaaccgaagcagacaagaaccatcgcgatttaatattaaggaagaggcaagtagcgggtctgagagtgggagcccaaaaagaagaggccagaggcagctgaaaaaacaagaaaaatggaaacaggaaccctcagaagatgaacaggaacaaggcaccagtgcagagagtgagccagaacaaaaaaaagtaaaagccagaagacctgtccccagaagaacagtgcccaaacctcgtgttaaaaagcagccgaagactcagcgtggaaagagaaaaaagcaagattcttctgatgaggatgatgatgatgacgaagctcccaaaaggcagactcgtcgaagagcggctaaaaacgttagttacaaagaagatgatgactttgagactgactcagatgatctcattgaaatgactggagaaggagttgatgaacagcaagataatagtgaaactattgaaaaggtcttagattcaagactgggaaagaaaggagccactggagcatctactactgtatatgcgattgaagctaatggcgaccctagtggtgactttgacactgaaaaggatgaaggtgaaatccagtacctcatcaagtggaagggttggtcttacatccacagcacatgggagagtgaagaatccttacagcaacagaaagtgaagggcctaaaaaaactagagaacttcaagaaaaaagaggacgaaatcaaacaatggttagggaaagtttctcctgaagatgtagaatatttcaattgccaacaggagctggcttcagagttgaataaacagtatcagatagtagaaagagtaatagctgtgaagacaagtaaatctacattgggtcaaacagattttccagctcatagtcggaagccggcaccctcaaatgagcccgaatatctatgtaaatggatgggactcccctattcagagtgtagctgggaagatgaagccctcattggaaagaaattccagaattgcattgacagcttccacagtaggaacaactcaaaaaccatcccaacaagagaatgcaaggccctgaagcagagaccacgatttgtagctttaaagaaacaacctgcatatttaggaggggagaatctggaacttcgagattatcagctagaaggtctaaactggctagctcattcctggtgcaaaaataatagtgtaatccttgctgatgaaatgggcctaggaaagaccatccagaccatatcattcctctcctacctgttccaccaacaccagctgtatggcccctttcttatagtcgtccctttatccaccctcacctcatggcagagagagtttgaaatctgggcaccagagattaacgtagtggtttacataggtgacctgatgagcagaaatacgatacgggaatatgaatggattcattcccaaaccaaaagattgaagttcaacgcacttataacaacatatgagatcctcttgaaagataagactgtgctgggcagtattaactgggcctttctgggagtggatgaagcccatcggttgaagaatgatgactctttattgtataaaactctgattgatttcaagtccaaccataggctcctgattacggggacccctcttcagaattccctcaaagagctctggtccttgctgcactttattatgccggagaagtttgaattttgggaagattttgaagaagaccatgggaaggggagagagaatggctaccagagtcttcataaggtgctagagcctttccttctccggagagtcaaaaaagatgtggagaaatcccttcctgctaaagtggaacagattctcagggtggagatgtcagcccttcagaaacagtattacaagtggattctgaccaggaattacaaggctcttgccaaaggaacaagaggcagcacatctggttttcttaatattgtgatggaactgaaaaaatgttgcaaccactgctatctgattaaaccccctgaagaaaatgaaagggaaaatggacaggagattcttctgtccctcataaggagcagtgggaagttgattttattagacaaactgttgacaagacttcgagaaagggggaatcgagtgcttatcttctctcagatggtgagaatgttggatatcctggctgaatacctaactattaaacactatcctttccagcgtctggatggttccatcaagggagaaatccgaaaacaggcactggaccacttcaatgcagatgggtctgaggacttctgtttcctgctctcgacaagggctggtggcctgggaatcaatttggcttcagcggacacagtcgtcatctttgactctgactggaacccccagaatgacttgcaggcacaagcccgagcgcatagaattggtcagaagaagcaggtaaatatttaccgcttagttacaaaggggactgtggaggaggagatcatagaacgggccaaaaagaagatggtattagatcatctggtgattcagcgcatggacaccactggccggacgatcctggaaaacaactcaggaaggtccaactcaaatccttttaataaagaagagctgacagctattttgaaatttggagcagaggatctcttcaaagaactggaaggggaggaatcagaacctcaggaaatggatatagatgaaattttgcggttggctgaaacgagagagaatgaagtgtcaacaagtgcaacagatgaacttctatcacagtttaaggttgccaactttgcaacaatggaagatgaagaagagctagaagagcgtcctcacaaggactgggatgagatcattccagaggaacaaaggaaaaaagtagaggaggaagagcggcagaaggagctagaagaaatttatatgctgcctcgaattcggagttccactaaaaaggctcagacaaatgacagtgactctgacactgagtctaagaggcaggcccagagatcctctgcttctgagagtgaaacggaagactctgatgatgacaagaagccaaagcgcagagggcgtccgaggagtgtgcggaaggacctcgtggagggatttactgatgcagagatccgaaggttcatcaaggcttataagaagtttggtctccctcttgaacggctggagtgcttagcacgtgatgctgagctggtagataagtcggtggcagatctgaagcgcctgggtgaactgatccacaacagctgtgtgtcagcaatgcaggaatatgaagagcagctgaaagaaaatgccagcgagggaaaaggaccagggaaaaggagaggtccaacaatcaagatatccggagttcaggttaatgtgaaatccattatccaacatgaagaggagtttgagatgctgcataaatctatccctgtggaccctgaagaaaaaaaaaaatactgcttaacctgtcgtgtcaaagctgcacattttgatgtagagtggggggtggaagatgattctcgcctgttgctggggatttatgaacatggctatggaaactgggagttaattaaaacagacccagagcttaaattaactgacaaaattctgccggtggagacagataaaaagcctcaggggaagcagctacagacccgagcggattacttgttgaagctgctcagaaagggtctggagaagaagggggctgtgacaggtggggaggaggccaaattaaagaagcggaagcctcgggtaaagaaggaaaacaaagtgcccaggctgaaagaggagcatggaattgagctttcatctcctaggcattcagataatccatcagaagagggagaagtgaaagatgatggcttggaaaaaagtccaatgaaaaaaaaacagaagaagaaagagaacaaggagaacaaggagaaacaaatgagttctaggaaagacaaagaaggggacaaggaaagaaagaagtcaaaagataagaaagagaagcctaaaagtggtgatgccaaatcttcgagtaaatcaaagcgatctcagggtcctgtccatattacagcaggaagtgaacctgtccccattggagaggatgaggatgatgatctggaccaggagacattcagcatatgtaaggagaggatgaggcccgtgaaaaaggcactgaaacagctcgacaaacctgacaaggggctcaacgtgcaagaacagctggaacacacccggaactgcctgctgaaaatcggagaccggatagccgagtgccttaaagcctactcagatcaggagcacatcaaactctggaggaggaacctatggatttttgtttccaagtttacagaatttgatgctcgaaaactgcataagttatacaagatggctcataagaaaaggtctcaagaagaagaggagcaaaagaagaaagacgacgtgactgggggtaagaaaccatttcgtccagaggcctcaggctccagccgggactctctgatatctcagtcccatacctcacacaaccttcaccctcagaagcctcatttgcctgcctcccatggcccacagatgcatggacacccaagagataactacaatcaccccaacaagagacacttcagtaatgcagatcgaggagactggcagagggaaagaaagttcaactatggtggtggcaacaacaatccaccatggggaagcgacaggcaccatcagtatgagcagcactggtacaaggaccaccattatggggaccggcgacatatggatgcccaccgttccggaagctatcgacccaacaacatgtccagaaagaggccttatgaccagtacagcagtgaccgagaccaccggggacacagagattattatgacaggtatgcaaaaggctgtgagacaccaggtgccaacctttgccaggagctgtttctagggagaaagtgacgtatacatgaatgtatttatctatcaaattactgaagatctcatcatgcatgtgtcagccacagcgaatcccatgtcttggttataggttttatgttttgttttctgggtcatagggagcacatttcacctgtgcaggaaaagagttttctgccgtcttttgaggaaatctagtgaagaggtcgccataaaatattagagtcaacaaccaaaattattaagctctgtgcgaggctgtcagccacactaggtatcagggatcccgagatgggtaccagcccacagtccttacctgccacgagcccataattgaagagtcaaagtcttctgaagctgcaccctctttacttcagtacaatgccaccagtagtacgatgagccaaagctttacattgtgagagtagcaagtccagggagagctaaagaggttttatctgtatttcctaatttcaaatcttggataatttaacctcatagcagctttggttttccctgggctgatgatgtgcgtcatttgcactgtaccttgaatttacagtgggaaaatttcatataaacgtgtcaaagtcgtgctttgtttttggaagatctggtaacagcagcccgcattagcagagagctgtagctgagtagctgccacctcgttgggagactgcccctcgctcccacccttctctattgtctggacccagtgggcatcttgccctgcgttcttctagtaggtctgtatttctatttgatgtcactttccttttgcctgaaggactttttctgctggtgataaactctttcagtgtttgtatatatgcctgaaaaagtattttgccttcatttttgaaagtagtttttgctgagtgtatacatttttggctttacagtttctttcagtgctttaaagatgtacctctgctatttacttgcattgttttgtgatgaaaaatctgtcatccttatctttgttcctctttacataatgttccttttaaaaaaaatcactgattatgatgtgccttggtgtatttttccttggtttcttgtgcttggaaatttttgaacttcttggatctgtgggtttattgtttccataaaatttggaaatttttacaatcttcttcaaatattttttctgatcccccactctctcttcttctttggagattctcattacacctatattagcttgcttgaagttgtctcacagctcacttgtattctgttgacttttaaaaaattatgctttctgtttcactgtggatagtttctattgctacctcttcaagttcactaatactttccttttcaatgtcaagactgctgtgaggcccatccagtgtactttgcattttatacattgtagttctaaaagttcggaaagttgtttttgggtctttttatatatgttctgtgtctaaccttttaaaacctggaacacagatataacaatggttttgatgtccttgtctgcgaatcttatcacttgggtcagtttcagttgatacctcctcactgtgggtcttgctcccctggtgctttctgtgcctagtaatttttgtcagatgccagatgtaacatttaccttgttgggtgctggatatttctgtattcctgtaagtattctggagctttgttatgagttgcaggttatttggaagcagtttcctttttcaggtcttgctgttaagattcgttaggtagaaccagagcagtgctcagtcaagggctaatgattgcccacccccaaggtaaagagcctcattgcactctacccaattgcgttagtctgttttgcaggaatacctgaggctgggtaatttatagagaaaagagttttatttgg

SEQ ID NO: 28 - DNA, 3001 bp, human
ggcagcgtccgcgggaggtgaggtggctgtggggacccaggtggcctcttccctggggccttgctaatgacggcaaaatccgggttctgccaaaatatatttaaaaaggtttattcctagtcagtatgagtgactgtggcccaggttattcagcctcaagaggtcctgtgaaagtgcccgagatggtcaggcttgcaggttaattttatacaattcagggagacaggaatttcaggtaaagtcataaatcaggctgagcagtgtggctcatgcctgtggtcccagcactttgggaggccaggagttccagagcagcctgggcagcacagcaagaccctgtctctacatgaaattagaaaaataaaaaaattagcggggcgtggtgtcccatgcctgtggcctcagctacttgggaggcccagtcagttgagtccaggaggtggaggctgtaaccagctatgttggctgcactgcacgctagcctgggtaacacagcgagatcctgcctccaaaaagaaaatcataaatcaataagagaaagatatacacgggttcctcccaaaaagctggtatatctccaaagggtttacacctcatgggggcacttagggattctttagtggacagttggttgagagacttaagctactgcctgaagactggaatcagaagcatgccagagttaaggggattgcgtagatcaaagttcttattatgtagatgaagcctcttagttggcaactctcagaatagatggtaaatgtctgttttcagttttttgggtttttgtgtttttgtttttgtttttagagagagtcttgctctgtcgcccaggctagagtgcagtggcgtgatctcagctcactgcaacctccacctcccaggtttgagcggttctcctgcctcggcctcctgggtagctgggactacgggcgcccgccaccacgcctggctaatttttgtatttttagtggagatggggtttcaccatgttgctgaggctggtcttgacttcctgacctcaggtgatccgcccacctctacctcccaaagtgctgggattacaggcgtgggccaccgcgcgtcaggctggctgtctcttccagacctaagaaaggcttagaacaaaggaggtctggctacattaatggagattcgctgcagatgcaaattttcccactaaagatagctttgcggggctatccatttcaatctgttgcccctgtggcagccacttcaaaacatgtcaaagaagtatattttggggtaaaataatttccttcagcatctgctgtcatgtgatgctgtaccagagtcaggttggaaagtgagcctcattatataagagtaataaaactcatctgatgagattttatggtttctcgggcaggattccccaagcctcatacataggcatttgggcaagggaaaaaaggtgaatttagtcctcaccaggttggtagggcttcctcggttattggagtgggagtaacagcaaccattgggcccagcagtttttttaaatgtctctggggctgtggactgaccatccaaataactgattttaatcatttcattatggaaaaattgtcagcagaacccccaagtagagagacccatcagtcaagatatacctcatgaccttgcaagctaatctagcttgacccagatcccctcctaatctgtgcagattcattgaggaatgtcatagccatgcctactggttaagacatagtcctttacagtgagagttgaaacccaagctctatcactttcttggctgtgttgctttgagaaaggcatttaaatgttttgtgcctgtttcctcatctgaaattggtgggtaatagtcacttcataggacagttgtgaagattgaatgcagaaaaatttgtgccacgcctggaaccgtccctggcatatattaaattctaaaaaagtgttaaatattataatgaatatcaacacttccttattctggaagcaccgacaggatatgctgtgtttagtgttagcatcatgtcaggacagggtctgttgcgatgcccacactcaggatctgttcccaggaacctgcgtaaagttttcttctctggaagactttgggtcctttttttttaacaagaagaggctctaccctgggactgggaatttccaaggccacctttgaggatcgcagagctcattttagagccattttagtccccagctcctcttcctccactcccacgttacccgtgagaggactgtctgcagggtaagggaggacagcccaaccccaggtggggacttcttatgtattgccttcctgcagtgccttctctgccctaaaccatggtgggtttcctttgctaatgtctgacatcttgtgccctacactgtcccatctgaggctcagaacctctcagccggttctcatggggaacgttccccagatctgatgccctcattcaggacacttccatcattgtccctacatttcttctctcagtgctttattcaggctgctgcattcgtggtgcagaccaggtcttgtaaaaaattattcagtcagcatgtgctgagccattgtcctgtcccagggacagggctttatagtcattgccctattcatctcttcaaccaatgtggaagttaggaattggaatccccatttcacagactaagaagtggcgtgttaatcagttgaaataatttttacggcttggcgtggtggcccatacctgtaatcccagcactttgggaggccggggcgggcggattacctgaggccaggggttcgagaccagcctggccaacatggtgaaacctcatctctgctgggaatacagaaattagccaggcatggtggctcacgcctgtagtcccaactgctctggagcctgaagcaggataatcgcttgaatccaggagatggaggttgcagtgagcagagagcatgccactgcactacagcctgagcaagagtgagactccgtcacaaaaaaaaaaaaaaaaaaac

SEQ ID NO: 29 - DNA, 24 bp, human
attattcaaggccgagtacagatg

SEQ ID NO: 30 - DNA, 23 bp, human
cacgtacacgatgtgtcccttct

SEQ ID NO: 31 - DNA, 21 bp, human
caggcggtgtgcctgctgcat

SEQ ID NO: 32 - DNA, 23 bp, human
tttgtggtgcctatttcaccttt

SEQ ID NO: 33 - DNA, 21 bp, human
cggagttccaagctgatggta

SEQ ID NO: 34 - DNA, 22 bp, human
ccacgtgtacggcttcggcctc

SEQ ID NO: 35 - DNA, 15 bp, human
ggcggagcgctacga

SEQ ID NO: 36 - DNA, 24 bp, human
ttcattcgagagaggttcattcag

SEQ ID NO: 37 - DNA, 21 bp, human
cctccgctatgaaggcggtga

SEQ ID NO: 38 - DNA, 22 bp, human
aagccaccccacttctctctaa

SEQ ID NO: 39 - DNA, 22 bp, human
aatgctatcacctcccctgtgt

SEQ ID NO: 40 - DNA, 26 bp, human
agaatggcccagtcctctcccaagtc

SEQ ID NO: 41 - DNA, 19 bp, human
cctgcccactgtgcttcct

SEQ ID NO: 42 - DNA, 19 bp, human
ggttttcccgcttgcagat

SEQ ID NO: 43 - DNA, 15 bp, human
ctggcttcaccatcg

SEQ ID NO: 44 - DNA, 21 bp, human
tggttggagagctcatttgga

SEQ ID NO: 45 - DNA, 22 bp, human
actctcgtcggtgactgttcag

SEQ ID NO: 46 - DNA, 16 bp, human
ttttgccgatttcatg

SEQ ID NO: 47 - DNA, 22 bp, human
cggaagaagaaacagctcatga

SEQ ID NO: 48 - DNA, 28 bp, human
cctctgtgtatttgtcaattttcttctc

SEQ ID NO: 49 - DNA, 17 bp, human
cggaaacaggccgagaa

SEQ ID NO: 50 - DNA, 18 bp, human
cctggcacccagcacaat

SEQ ID NO: 51 - DNA, 21 bp, human
gccgatccacacggagtactt

SEQ ID NO: 52 - DNA, 27 bp, human
atcaagatcattgctcctcctgagcgc

SEQ ID NO: 53 - DNA, 22 bp, human
gcctactttccaagcggagcca

SEQ ID NO: 54 - DNA, 19 bp, human
ttgcgggtacccacgcgaa

SEQ ID NO: 55 - DNA, 29 bp, human
aacggcaatgcggctgcaacggcggaatt

SEQ ID NO: 56 - DNA, 29 bp, human
caacctgtcagatacaatagaaggagtaa

SEQ ID NO: 57 - DNA, 21 bp, human
gcaaccagggtaatcgcagta

SEQ ID NO: 58 - DNA, 27 bp, human
gcccgatttggagaaacgacgcatctt

SEQ ID NO: 59 - DNA, 19 bp, human
gcagtacgccccgaacact

SEQ ID NO: 60 - DNA, 24 bp, human
aaaattgcttgaagatgggactct

SEQ ID NO: 61 - DNA, 23 bp, human
tggagattctgcctcagggccgt

SEQ ID NO: 62 - DNA, 20 bp, human
ccctggaactcatggtctca

SEQ ID NO: 63 - DNA, 20 bp, human
cgagaccccaatcaaaacct

SEQ ID NO: 64 - DNA, 23 bp, human
cagggccgccctccacacctgtt

SEQ ID NO: 65 - DNA, 15 bp, human
ccaccggacgccatc

SEQ ID NO: 66 - DNA, 20 bp, human
ttctcgtagctcgccacact

SEQ ID NO: 67 - DNA, 21 bp, human
tcccggcgggattctgatgtt

SEQ ID NO: 68 - DNA, 17 bp, human
gcctccgctatgaaggc

SEQ ID NO: 69 - DNA, 20 bp, human
atcaagatcattgctcctcc

SEQ ID NO: 70 - DNA, 17 bp, human
tggagattctgcctcag

SEQ ID NO: 71 - DNA, 25 bp, human
gcccgatttggagaaacgacgcatc

SEQ ID NO: 72 - DNA, 25 bp, human
tgaacagtcaccgacgagagtgctg

SEQ ID NO: 73 - DNA, 20 bp, human
gtcccggcgggattctgatg

SEQ ID NO: 74 - DNA, 27 bp, human
aacggcaatgcggctgcaacggcggaa

SEQ ID NO: 75 - DNA, 22 bp, human
gcctccgctatgaaggcggtga

SEQ ID NO: 76 - DNA, 19 bp, human
cggaaacaggccgagaatt

SEQ ID NO: 77 - DNA, 18 bp, human
ttttgccgatttcatgtt

SEQ ID NO: 78 - DNA, 23 bp, human
caggcggtgtgcctgctgcattt

SEQ ID NO: 79 - DNA, 22 bp, human
gtcccggcgggattctgatgtt

SEQ ID NO: 80 - DNA, 60 bp, human
aaacgacgcatccactactgcgattaccctggttgcacaaaagtttataccaagtcttct

SEQ ID NO: 81 - DNA, 24 bp, human
catttaaaagctcacctgaggact

SEQ ID NO: 82 - DNA, 24 bp, human
catttaaaagctcacctgaggact

SEQ ID NO: 83 - DNA, 103 bp, human
gaattcgcccttgggctctgtggcaagatctatatctggaaggggcgaaaagcgaatgagaaggagcggcaagggcgaattcgtttaaacctgcaggactagt

SEQ ID NO: 84 - DNA, 59 bp, human
gggctctgtggcaagatctatatctggaaggggcgaaaagcgaatgagaaggagcggca

SEQ ID NO: 85 - DNA, 59 bp, human; identical to SEQ ID NO: 84

SEQ ID NO: 86 - DNA, 106 bp, human
gaattcgcccttccctggcatccgagacagtgccttctccatggagtccattgatgattacgtgaacgttccgaagggcgaattcgtttaaacctgcaggactagt

SEQ ID NO: 87 - DNA, 60 bp, human
ccctggcatccgagacagtgccttctccatggagtccattgatgattacgtgaacgttcc

SEQ ID NO: 88 - DNA, 60 bp, human; identical to SEQ ID NO: 87

SEQ ID NO: 89 - DNA, 123 bp, human
gaattcgcccttccaatcaaaacctccaggtatcttcccagactaggtgtggagggcggccctgtgggtgggaggctggagcctccagagtgtcctgagaccatgagttccaagggcgaattc

SEQ ID NO: 90 - DNA, 60 bp, human
ccaatcaaaacctccaggtatcttcccagactaggtgtggagggcggccctgtgggtggg

SEQ ID NO: 91 - DNA, 60 bp, human
ccaatcaaaacctccaggtatcttcccagaccaggtgtggagggcggccctgtgggtggg

SEQ ID NO: 92 - DNA, 45 bp, human
aggctggagcctccagagtgtcctgagaccatgagttccaagggc

SEQ ID NO: 93 - DNA, 45 bp, human
aggctggagcctccagagtgtcctgagaccatgagttccaggggc

SEQ ID NO: 94 - DNA, 17 bp, human
ccacgtgtacggcttcg

SEQ ID NO: 95 - DNA, 22 bp, human
ccacgtgtacggcttcggcctc

SEQ ID NO: 96 - DNA, 15 bp, human
ggcggagcgctacga

SEQ ID NO: 97 - DNA, 26 bp, human
ttcattcgagagaggttcattcagvd

(19) United States
(12) Patent Application Publication
(10) Pub. No.: US 2008/0058432 A1
(43) Pub. Date: Mar. 6, 2008

(54) Molecular assay to predict recurrence of Duke's B colon cancer

(76) Inventors: Yixin Wang, San Diego, CA (US); Abhijit Mazumder, Basking Ridge, NJ (US); Yuqiu Jiang, San Diego, CA (US); Thomas Briggs, Franklinton, NC (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/714,755

(22) Filed: Mar. 5, 2007

Related U.S. Application Data
(60) Provisional application No. 60/779,170, filed on Mar. 3, 2006.

(51) Int. Cl. C12Q 1/68; A61K 45/00; C40B 30/00; C40B 40/08; C40B 30/04; A61P 35/00

(52) U.S. Cl. 514/789; 435/6; 506/17; 506/7; 506/9

(57) ABSTRACT

A method of providing a prognosis of colorectal cancer is conducted by analyzing the expression of a group of genes. Gene expression profiles in a variety of medium such as microarrays are included as are kits that contain them.

### BACKGROUND

This invention relates to prognostics for colorectal cancer based on the gene expression profiles of biological samples.

Colorectal cancer is a heterogeneous disease with complex origins. Once a patient is treated for colorectal cancer, the likelihood of a recurrence is related to the degree of tumor penetration through the bowel wall and the presence or absence of nodal involvement. These characteristics are the basis for the current staging system defined by Duke's classification. Duke's A disease is confined to submucosa layers of colon or rectum. Duke's B tumor invades through muscularis propria and could penetrate the wall of colon or rectum. Duke's C disease includes any degree of bowel wall invasion with regional lymph node metastasis.

Surgical resection is highly effective for early stage colorectal cancers, providing cure rates of 95% in Duke's A and 75% in Duke's B patients. The presence of positive lymph node in Duke's C disease predicts a 60% likelihood of recurrence within five years. Treatment of Duke's C patients with a post surgical course of chemotherapy reduces the recurrence rate to 40%-50%, and is now the standard of care for Duke's C patients. Because of the relatively low rate of reoccurrence, the benefit of post surgical chemotherapy in Duke' B has been harder to detect and remains controversial. However, the Duke's B classification is imperfect as approximately 20-30% of these patients behave more like Duke's C and relapse within a 5-year timeframe.

There is clearly a need to identify better prognostic factors than nodal involvement for guiding selection of Duke's B into those that are likely to relapse and those that will survive. Rosenwald et al. (2002); Compton et al. (2000); Ratto et al. (1998); Watanabe et al. (2001); Noura et al. (2002); Halling et al. (1999); Martinez-Lopez, et al. (1998); Zhou et al. (2002); Ogunbiyi et al. (1998); Shibata et al. (1996); Sun et al. (1999); and McLeod et al. (1999). This information would allow better informed planning by identifying patients who are more likely to require and possibly benefit from adjuvant therapy. Johnston (2005); Saltz et al. (1997); Wolmark et al. (1999); International multicenter pooled analysis of B2 colon cancer trials (IMPACT B2) investigators: Efficacy of adjuvant fluorouracil and folinic acid in B2 colon cancer (1999); and Mamounas et al. (1999).

The clinical application of genomics in the diagnosis and management of cancer is gaining momentum as discovery and initial validation studies are completed. Allen et al. (2005a); Allen et al. (2005b); Van't Veer et al. (2002); Van de Vijver et al. (2002); Wang et al (2005); Beer et al. (2002); and Shipp et al. (2002). As more studies are published there has been an increasing appreciation of the challenges facing the implementation of these signatures in general clinical practice. Ransohoff (2005) and Simon et al. (2003) have recently described the merit of elimination of bias and critical aspects of molecular marker evaluation. A common unambiguous requirement for broader acceptance of a molecular signature is the validation of the assay performance on a truly independent patient population. An additional limitation is that the DNA microarray-based assays require fresh frozen tissue samples. As a result, these tests cannot readily be applied to standard clinical material such as frozen paraffin embedded (FPE) tissues samples.

In commonly owned US published Patent Applications 20050048526, 20050048494, 20040191782, 20030186303 and 20030186302 and Wang et al. (2005) gene expression profiles prognostic for colon cancer were presented. This specification presents materials and methods for determining gene expression profiles.

### SUMMARY OF THE INVENTION

The invention provides materials and methods for assessing the likelihood of a recurrence of colorectal cancer in a patient diagnosed with or treated for colorectal cancer. The method involves the analysis of a gene expression profile.

In one aspect of the invention, the gene expression profile includes primers and probes for detecting expression of at least seven particular genes.

Articles used in practicing the methods are also an aspect of the invention.

Such articles include gene expression profiles or representations of them that are fixed in machine-readable media such as computer readable media.

Articles used to identify gene expression profiles can also include substrates or surfaces, such as microarrays, to capture and/or indicate the presence, absence, or degree of gene expression.

In yet another aspect of the invention, kits include reagents for conducting the gene expression analysis prognostic of colorectal cancer recurrence.

### BRIEF DESCRIPTION OF THE DRAWINGS

FIG. 1 is a standard Kaplan-Meier Plot constructed from the independent patient data set of 27 patients (14 survivors, 13 relapses) as described in the Examples for the analysis of the seven gene portfolio. Two classes of patients are indicated as predicted by chip data. The vertical axis shows the probability of disease-free survival among patients in each class.

FIG. 2 is a standard Kaplan-Meier Plot constructed from the independent patient data set of 9 patients (6 survivors, 3 relapses) as described in the Examples for the analysis of the 15 gene portfolio. Two classes of patients are indicated as predicted by chip data. The vertical axis shows the probability of disease-free survival among patients in each class.

FIG. 3 is a standard Kaplan-Meier Plot constructed from patient data as described in the Examples and using the 22-gene profile with the inclusion of Cadherin 17 (SEQ ID NO: 6) to the portfolio. Thirty-six samples were tested (20 survivor, 16 relapses) Two classes of patients are indicated as predicted by chip data of the 23-gene panel. The vertical axis shows the probability of disease-free survival among patients in each class.

FIG. 4 is a ROC and Kaplan-Meier survival analysis of the prognostic signatures on 123 independent patients. A. The ROC curve of the gene signature. B. Kaplan-Meier curve and log rank test of 123 frozen tumor samples. The risk of recurrence for each patient was assessed based on the gene signature and the threshold was determined by the training set. The high and low risk groups differ significantly (P=0.04).

FIG. 5 is a ROC and Kaplan-Meier survival analysis of the prognostic signatures on 110 independent patients. A. The ROC curve of the gene signature. B. Kaplan-Meier curve and log rank test of 110 FPE tumor samples. The risk of recurrence for each patient was assessed based on the gene signature and the threshold was determined by the training set. The high and low risk groups differ significantly (P<0.0001).

FIG. 6 is an electrophoretogram.

### DETAILED DESCRIPTION

A Biomarker is any indicia of the level of expression of an indicated Marker gene. The indicia can be direct or indirect and measure over- or under-expression of the gene given the physiologic parameters and in comparison to an internal control, normal tissue or another carcinoma. Biomarkers include, without limitation, nucleic acids (both over and under-expression and direct and indirect). Using nucleic acids as Biomarkers can include any method known in the art including, without limitation, measuring DNA amplification, RNA, micro RNA, loss of heterozygosity (LOH), single nucleotide polymorphisms (SNPs, Brookes (1999)), microsatellite DNA, DNA hypo- or hyper-methylation. Using proteins as Biomarkers includes any method known in the art including, without limitation, measuring amount, activity, modifications such as glycosylation, phosphorylation, ADP-ribosylation, ubiquitination, etc., or imunohistochemistry (IHC). Other Biomarkers include imaging, cell count and apoptosis Markers.

The indicated genes provided herein are those associated with a particular tumor or tissue type. A Marker gene may be associated with numerous cancer types but provided that the expression of the gene is sufficiently associated with one tumor or tissue type to be identified using the methods described herein and those known in the art to predict recurrence of Duke's B colon cancer. The present invention provides preferred Marker genes and even more preferred Marker gene combinations. These are described herein in detail.

A Marker gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with a tumor or tissue type. The preferred Marker genes are those associated with SEQ ID NOs: 7-28. The polynucleotide primers and probes of the invention are shown as SEQ ID NOs: 29-79 and 94-97. The amplicons of the present invention are shown as SEQ ID NOs: 5-6, 80-93.

Amplicons

| | SEQ |
|---|---|
| Sequence | ID NO |
| GAATTCGCCCTTGAGAAAACGACGCATCCACTACTGCGATTACC | 5 |
| CTGGTTGCACAAAAGTTTACACCAAGTCTTCTCATTTAAAAGCT | |
| CACCTGAGGACTAAGGGCGAATTC | |
| AAACGACGCATCCACTACTGCGATTACCCTGGTTGCACAAAAG | 6 |
| TTTACACCAAGTCTTCT | |
| AAACGACGCATCCACTACTGCGATTACCCTGGTTGCACAAAAGT | 80 |
| TTATACCAAGTCTTCT | |
| CATTTAAAAGCTCACCTGAGGACT | 81 |
| CATTTAAAAGCTCACCTGAGGACT | 82 |
| GAATTCGCCCTTGGGCTCTGTGGCAAGATCTATATCTGGAAGGG | 83 |
| GCGAAA□AGCGAATGAGAAGGAGCGGCAAGGGCGAATTCGTTTA | |
| AACCTGCAGGACT□AGT | |
| GGGCTCTGTGGCAAGATCTATATCTGGAAGGGGCGAAAAGCGAA | 84 |
| TGAGAAGGAGCGGCA | |
| GGGCTCTGTGGCAAGATCTATATCTCGAAGCGGCGAAAAGCGAA | 85 |
| TGAGAAGGAGCGGCA | |
| GAATTCGCCCTTCCCTGGCATCCGAGACAGTGCCTTCTCCATGG | 86 |
| AGTCCATTGATGATTACGTGAACGTTCCGAAGGGCGAATTCGTT | |
| TAAACCTGCAGGACTAGT | |
| CCCTGGCATCCGAGACAGTGCCTTCTCCATGGAGTCCATTGATG | 87 |
| ATTACGTGAACGTTCC | |
| CCCTGGCATCCGAGACAGTGCCTTCTCCATGGAGTCCATTGATG | 88 |
| ATTACGTGAACGTTCC | |
| GAATTCGCCCTTCCAATCAAAACCTCCAGGTATCTTCCCAGACT | 89 |
| AGGTGTGGAGGGCGGCCCTGTGGGTGGGAGGCTGGAGCCTCCAG | |
| AGTGTCCTGAGACCATGAGTTCCAAGGGCGAATTC | |
| CCAATCAAAACCTCCAGGTATCTTCCCAGACTAGGTGTGGAGGG | 90 |
| CGGCCCTGTGGGTGGG | |
| CCAATCAAAACCTCCAGGTATCTTCCCAGACCAGGTGTGGAGGG | 91 |
| CGGCCCTGTGGGTGGG | |
| AGGCTGGAGCCTCCAGAGTGTCCTGAGACCATGAGTTCCAAGGG | 92 |
| C | |
| AGGCTGGAGCCTCCAGAGTGTCCTGAGACCATGAGTTCCAGGGG | 93 |
| C | |

In one embodiment the Marker genes are those associated with any one of SEQ ID NOs: 7-28. In another embodiment, the polynucleotide primers and probes of the invention are at least one of SEQ ID NOs: 29-79 and 94-97. In another embodiment, the Markers are identified by the production of at least one of the amplicons SEQ ID NOs: 5-6, 80-93. The present invention further provides kits for conducting an assay according to the methods provided herein and further containing Biomarker detection reagents.

The present invention further provides microarrays or gene chips for performing the methods described herein.

The present invention provides methods of obtaining additional clinical information including obtaining optimal biomarker sets for carcinomas; providing direction of therapy and identifying the appropriate treatment therefor; and providing a prognosis.

The present invention further provides methods of finding Biomarkers by determining the expression level of a Marker gene in a particular metastasis, measuring a Biomarker for the Marker gene to determine expression thereof, analyzing the expression of the Marker gene according to any of the methods provided herein or known in the art and determining if the Marker gene is effectively specific for the prognosis.

The present invention further provides diagnostic/prognostic portfolios containing isolated nucleic acid sequences, their complements, or portions thereof of a combination of genes as described herein where the combination is sufficient to measure or characterize gene expression in a biological sample having metastatic cells relative to cells from different carcinomas or normal tissue.

Any method described in the present invention can further include measuring expression of at least one gene constitutively expressed in the sample.

The mere presence or absence of particular nucleic acid sequences in a tissue sample has only rarely been found to have diagnostic or prognostic value. Information about the expression of various proteins, peptides or mRNA, on the other hand, is increasingly viewed as important. The mere presence of nucleic acid sequences having the potential to express proteins, peptides, or mRNA (such sequences referred to as “genes”) within the genome by itself is not determinative of whether a protein, peptide, or mRNA is expressed in a given cell. Whether or not a given gene capable of expressing proteins, peptides, or mRNA does so and to what extent such expression occurs, if at all, is determined by a variety of complex factors. Irrespective of difficulties in understanding and assessing these factors, assaying gene expression can provide useful information about the occurrence of important events such as tumorogenesis, metastasis, apoptosis, and other clinically relevant phenomena. Relative indications of the degree to which genes are active or inactive can be found in gene expression profiles. The gene expression profiles of this invention are used to provide a diagnosis and treat patients.

Sample preparation requires the collection of patient samples. Patient samples used in the inventive method are those that are suspected of containing diseased cells such as cells taken from a nodule in a fine needle aspirate (FNA) of tissue. Bulk tissue preparation obtained from a biopsy or a surgical specimen and laser capture microdissection are also suitable for use. Laser Capture Microdissection (LCM) technology is one way to select the cells to be studied, minimizing variability caused by cell type heterogeneity. Consequently, moderate or small changes in Marker gene expression between normal or benign and cancerous cells can be readily detected. Samples can also comprise circulating epithelial cells extracted from peripheral blood. These can be obtained according to a number of methods but the most preferred method is the magnetic separation technique described in U.S. Pat. No. 6,136,182. Once the sample containing the cells of interest has been obtained, a gene expression profile is obtained using a Biomarker, for genes in the appropriate portfolios.

Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in for instance, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756;

5,545,531; 5,554,501; 5,561,071; 5,571,639; 5,593,839; 5,599,695; 5,624,711; 5,658,734; and 5,700,637.

Microarray technology allows for measuring the steady-state mRNA level of thousands of genes simultaneously providing a powerful tool for identifying effects such as the onset, arrest, or modulation of uncontrolled cell proliferation. Two microarray technologies are currently in wide use, cDNA and oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. No. 6,271,002; 6,218,122; 6,218,114; and 6,004,755.

Analysis of the expression levels is conducted by comparing such signal intensities. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

The selection can be based on statistical tests that produce ranked lists related to the evidence of significance for each gene's differential expression between factors related to the tumor's prognosis. Examples of such tests include ANOVA and Kruskal-Wallis. The rankings can be used as weightings in a model designed to interpret the summation of such weights, up to a cutoff, as the preponderance of evidence in favor of one class over another. Previous evidence as described in the literature may also be used to adjust the weightings.

A preferred embodiment is to normalize each measurement by identifying a stable control set and scaling this set to zero variance across all samples. This control set is defined as any single endogenous transcript or set of endogenous transcripts affected by systematic error in the assay, and not known to change independently of this error. All markers are adjusted by the sample specific factor that generates zero variance for any descriptive statistic of the control set, such as mean or median, or for a direct measurement. Alternatively, if the premise of variation of controls related only to systematic error is not true, yet the resulting classification error is less when normalization is performed, the control set will still be used as stated. Non-endogenous spike controls could also be helpful, but are not preferred.

Gene expression profiles can be displayed in a number of ways. The most common is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (down-regulation) appears in the blue portion of the spectrum while a ratio greater than one (up-regulation) appears in the red portion of the spectrum. Commercially available computer software programs are available to display such data including “GeneSpring” (Silicon Genetics, Inc.) and “Discovery” and “Infer” (Partek, Inc.)

Measurements of the abundance of unique RNA species are collected from primary tumors or metastatic tumors. These readings along with clinical records including, but not limited to, a patient's age, gender, site of origin of primary tumor, and site of metastasis (if applicable) are used to generate a relational database. The database is used to select RNA transcripts and clinical factors that can be used as marker variables to predict the risk of relapse of a tumor.

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

Modulated genes used in the methods of the invention are described in the Examples. The genes that are differentially expressed are either up regulated or down regulated in patients with recurrence versus those without recurrence of Dukes' B colon cancer. Up regulation and down regulation are relative terms meaning that a detectable difference (beyond the contribution of noise in the system used to measure it) is found in the amount of expression of the genes relative to some baseline. In this case, the baseline is determined based on the classification tree. The genes of interest in the diseased cells are then either up regulated or down regulated relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis may include the determination of disease/status issues such as determining the likelihood of relapse, type of therapy and therapy monitoring. In therapy monitoring, clinical judgments are made regarding the effect of a given course of therapy by comparing the expression of genes over time to determine whether the gene expression profiles have changed or are changing to patterns more consistent with normal tissue.

Genes can be grouped so that information obtained about the set of genes in the group provides a sound basis for making a clinically relevant judgment such as a diagnosis, prognosis, or treatment choice. These sets of genes make up the portfolios of the invention. As with most diagnostic Markers, it is often desirable to use the fewest number of Markers sufficient to make a correct medical judgment. This prevents a delay in treatment pending further analysis as well unproductive use of time and resources.

One method of establishing gene expression portfolios is through the use of optimization algorithms such as the mean variance algorithm widely used in establishing stock portfolios. This method is described in detail in 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Markowitz (1952). Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional Markers such as serum protein Markers (e.g., Cancer Antigen 27.29 (“CA 27.29”)). A range of such Markers exists including such analytes as CA 27.29. In one such method, blood is periodically taken from a treated patient and then subjected to an enzyme immunoassay for one of the serum Markers described above. When the concentration of the Marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Kits made according to the invention include formatted assays for determining the gene expression profiles. These can include all or some of the materials needed to conduct the assays such as reagents and instructions and a medium through which Biomarkers are assayed.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The following examples are provided to illustrate but not limit the claimed invention. All references cited herein are hereby incorporated herein by reference.

The preferred profiles of this invention are the seven-gene portfolio shown in Table 2 and the fifteen-gene portfolio shown in Table 3. Gene expression portfolios made up another independently verified colorectal prognostic gene such as Cadherin 17 together with the combination of genes in both Table 2 and Table 3 are most preferred (Table 4). This most preferred portfolio best segregates Duke's B patients at high risk of relapse from those who are not. Once the high-risk patients are identified they can then be treated with adjuvant therapy. Other independently verified prognostic genes can be used in place of Cadherin 17.

In this invention, the most preferred method for analyzing the gene expression pattern of a patient to determine prognosis of colon cancer is through the use of a Cox hazard analysis program. Most preferably, the analysis is conducted using S-Plus software (commercially available from Insightful Corporation). Using such methods, a gene expression profile is compared to that of a profile that confidently represents relapse (i.e., expression levels for the combination of genes in the profile is indicative of relapse). The Cox hazard model with the established threshold is used to compare the similarity of the two profiles (known relapse versus patient) and then determines whether the patient profile exceeds the threshold. If it does, then the patient is classified as one who will relapse and is accorded treatment such as adjuvant therapy. If the patient profile does not exceed the threshold then they are classified as a non-relapsing patient. Other analytical tools can also be used to answer the same question such as, linear discriminate analysis, logistic regression and neural network approaches.

Numerous other well-known methods of pattern recognition are available. The following references provide some examples:
Weighted Voting: Golub et al. (1999).
Support Vector Machines and K-nearest Neighbors: Su et al. (2001); and Ramaswamy et al. (2001).
Correlation Coefficients: van 't Veer et al. (2002) Gene expression profiling predicts clinical outcome of breast cancer Nature 415:530-536.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional markers such as serum protein markers (e.g., carcinoembryonic antigen). A range of such markers exists including such analytes as CEA. In one such method, blood is periodically taken from a treated patient and then subjected to an enzyme immunoassay for one of the serum markers described above. When the concentration of the marker suggests the return of tumors or failure of therapy, a sample source amenable to gene expression analysis is taken. Where a suspicious mass exists, a fine needle aspirate is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting colorectal cancer.

Kits made according to the invention include formatted assays for determining the gene expression profiles. These can include all or some of the materials needed to conduct the assays such as reagents and instructions.

Primers and probes useful in the invention include, without limitation, one or several of the following:

SEQ ID NO: 29

| | |
|---|---|
| Laforin forward, | cattattcaaggccgagtacagatg; |

SEQ ID NO: 30

| | |
|---|---|
| Laforin reverse, | cacgtacacgatgtgtcccttct; |

SEQ ID NO: 31

| | |
|---|---|
| Laforin probe, | caggcggtgtgcctgctgcat; |

SEQ ID NO: 32

| | |
|---|---|
| RCC1 forward, | tttgtggtgcctatttcaccttt; |

SEQ ID NO: 33

| | |
|---|---|
| RCC1 reverse, | cggagttccaagctgatggta; |

SEQ ID NO: 34

| | |
|---|---|
| RCC1 probe, | ccacgtgtacggcttcggcctc. |

SEQ ID NO: 35

| | |
|---|---|
| YWHAH forward, | ggcggagcgctacga; |

SEQ ID NO: 36

| | |
|---|---|
| YWHAH reverse, | ttcattcgagagaggttcattcag; |

SEQ ID NO: 37

| | |
|---|---|
| YWHAH probe, | cctccgctatgaaggcggtgã; |

SEQ ID NO: 38

| | |
|---|---|
| β-actin forward, | aagccaccccacttctctctaa; |

SEQ ID NO: 39

| | |
|---|---|
| β-actin reverse, | aatgctatcacctcccctgtgt; |

SEQ ID NO: 40

| | |
|---|---|
| β-actin probe, | agaatggcccagtcctctcccaagtc. |

SEQ ID NO: 41

| | |
|---|---|
| HMBS forward, | cctgcccactgtgcttcct; |

SEQ ID NO: 42

| | |
|---|---|
| HMBS reverse, | ggttttcccgcttgcagat; |

SEQ ID NO: 43

| | |
|---|---|
| HMBS probe, | ctggcttcaccatcg. |

SEQ ID NO: 44

| | |
|---|---|
| GUSB forward, | tggttggagagctcatttgga; |

SEQ ID NO: 45

| | |
|---|---|
| GUSB reverse, | actctcgtcggtgactgttcag; |

SEQ ID NO: 46

| | |
|---|---|
| GUSB probe, | ttttgccgatttcatg. |

SEQ ID NO: 47

| | |
|---|---|
| RPL13A forward, | cggaagaagaaacagctcatga; |

SEQ ID NO: 48

| | |
|---|---|
| RPL13A reverse, | cctctgtgtatttgtcaattttcttctc; |

SEQ ID NO: 49

| | |
|---|---|
| RPL13A probe, | cggaaacaggccgagaa. |

These primers and probes can include about 1-5 bases both 5′ and 3′ based on the known sequences of the subject genes. Preferably, the primer and probe sets are used together to measure the expression of the subject gene in a PCR reaction.

The invention is further illustrated by the following non-limiting examples. All references cited herein are hereby incorporated herein by reference.

### EXAMPLES

Genes analyzed according to this invention are typically related to full-length nucleic acid sequences that code for the production of a protein or peptide. One skilled in the art will recognize that identification of full-length sequences is not necessary from an analytical point of view. That is, portions of the sequences or ESTs can be selected according to well-known principles for which probes can be designed to assess gene expression for the corresponding gene.

### Example 1

Sample Handling and LCM.

Fresh frozen tissue samples were collected from patients who had surgery for colorectal tumors. The samples that were used were from 63 patients staged with Duke's B according to standard clinical diagnostics and pathology. Clinical outcome of the patients was known. Thirty-six of the patients have remained disease-free for more than 3 years while 27 patients had tumor relapse within 3 years.

The tissues were snap frozen in liquid nitrogen within 20-30 minutes of harvesting, and stored at −80 C° thereafter. For laser capture, the samples were cut (6 μm), and one section was mounted on a glass slide, and the second on film (P.A.L.M.), which had been fixed onto a glass slide (Micro Slides Colorfrost, VWR Scientific, Media, Pa.). The section mounted on a glass slide was after fixed in cold acetone, and stained with Mayer's Haematoxylin (Sigma, St. Louis, Mo.). A pathologist analyzed the samples for diagnosis and grade. The clinical stage was estimated from the accompanying surgical pathology and clinical reports to verify the Dukes classification. The section mounted on film was after fixed for five minutes in 100% ethanol, counter stained for 1 minute in eosin/100% ethanol (100 μg of Eosin in 100 ml of dehydrated ethanol), quickly soaked once in 100% ethanol to remove the free stain, and air dried for 10 minutes.

Before use in LCM, the membrane (LPC-MEMBRANE PEN FOIL 1.35 μm No 8100, P.A.L.M. GmbH Mikrolaser Technologie, Bernried, Germany) and slides were pretreated to abolish RNases, and to enhance the attachment of the tissue sample onto the film. Briefly, the slides were washed in DEP H₂O, and the film was washed in RNase AWAY (Molecular Bioproducts, Inc., San Diego, Calif.) and rinsed in DEP H₂O. After attaching the film onto the glass slides, the slides were baked at +120° C. for 8 hours, treated with TI-SAD (Diagnostic Products Corporation, Los Angeles, Calif., 1:50 in DEP H₂O, filtered through cotton wool), and incubated at +37° C. for 30 minutes. Immediately before use, a 10 μl aliquot of RNase inhibitor solution (Rnasin Inhibitor 2500 U=33 U/μl N211A, Promega GmbH, Mannheim, Germany, 0.5 μl in 400 μl of freezing solution, containing 0.15 M NaCl, 10 mM Tris pH 8.0, 0.25 mmol dithiothreitol) was spread onto the film, where the tissue sample was to be mounted.

The tissue sections mounted on film were used for LCM. Approximately 2000 epithelial cells/sample were captured using the PALM Robot-Microbeam technology (P.A.L.M. Mikrolaser Technologie, Carl Zeiss, Inc., Thomwood, N.Y.), coupled into Zeiss Axiovert 135 microscope (Carl Zeiss Jena GmbH, Jena, Germany). The surrounding stroma in the normal mucosa, and the occasional intervening stromal components in cancer samples, were included. The captured cells were put in tubes in 100% ethanol and preserved at −80° C.

### Example 2

RNA Extraction and Amplification.

Zymo-Spin Column (Zymo Research, Orange, Calif. 92867) was used to extract total RNA from the LCM captured samples. About 2 ng of total RNA was resuspended in 10 μl of water and 2 rounds of the T7 RNA polymerase based amplification were performed to yield about 50 Vg of amplified RNA.

### Example 3

DNA Microarray Hybridization and Quantitation.

A set of DNA microarrays consisting of approximately 23,000 human DNA clones was used to test the samples by use of the human U133a chip obtained and commercially available from Affymetrix, Inc. Total RNA obtained and prepared as outlined above and applied to the chips and analyzed by Agilent BioAnalyzer according to the manufacturer's protocol. All 63 samples passed the quality control standards and the data were used for marker selection.

Chip intensity data was analyzed using MAS Version 5.0 software commercially available from Affymetrix, Inc. (“MAS 5.0”). An unsupervised analysis was used to identify two genes that distinguish patients that would relapse from those who would not as follows.

The chip intensity data obtained as described was the input for the unsupervised clustering software commercially available as PARTEK version 5.1 software. This unsupervised clustering algorithm identified a group of 20 patients with a high frequency of relapse (13 relapsers and 7 survivors). From the original 23,000 genes, the-testing analysis selected 276 genes that significantly differentially expressed in these patients. From this group, two genes were selected that best distinguish relapsing patients from those that do not relapse: Human intestinal peptide-associated transporter (SEQ ID NO: 3) and Homo sapiens fatty acid binding protein 1 (SEQ ID NO: 1). These two genes are down-regulated (in fact, they are turned off or not expressed) in the relapsing patients from this patient group.

Supervised analysis was then conducted to further discriminate relapsing patients from those who did not relapse in the remaining 43 patients. This group of patient data was then divided into the following groups: 27 patients were assigned as the training set and 16 patients were assigned as the testing set. This ensured that the same data was not used to both identify markers and then validate their utility.

An unequal variance t-test was performed on the training set. From a list of 28 genes that have significant corrected p values, MHC II-DR-B was chosen. These genes are down-regulated in relapsers. MHC II-DR-B (SEQ ID NO: 2) also had the smallest p-value.

In an additional round of supervised analysis, a variable selection procedure for linear discriminant analysis was implemented using the Partek Version 5.0 software described above to separate relapsers from survivors in the training set. The search method was forward selection. The variable selected with the lowest posterior error was immunoglobulin-like transcript 5 protein (SEQ ID NO: 4). A Cox proportional hazard model (using “S Plus” software from Insightful, Inc.) was then used for gene selection to confirm gene selection identified above for survival time. In each cycle of total 27 cycles, each of the 27 patients in the training set was held out, the remaining 26 patients were used in the univariate Cox model regression to assess the strength of association of gene expression with the patient survival time. The strength of such association was evaluated by the corresponding estimated standardized parameter estimate and P value returned from the Cox model regression. P value of 0.01 was used as the threshold to select top genes from each cycle of the leave-one-out gene selection. The top genes selected from each cycle were then compared in order to select those genes that showed up in at least 26 times in the total of 27 leave-one-out gene selection cycles. A total of 70 genes were selected and both MHC II-DR-B and immunoglobulin-like transcript 5 protein were among them (Again, showing down regulation).

Construction of a multiple-gene predictor: Two genes, MHC II-DR-B and immunoglobulin-like transcript 5 protein were used to produce a predictor using linear discriminant analysis. The voting score was defined as the posterior probability of relapse. If the patient score was greater than 0.5, the patient was classified as a relapser. If the patient score was less than 0.5, the patient was classified as a survivor. The predictor was tested on the training set.

Cross-validation and evaluation of predictor: Performance of the predictor should be determined on an independent data set because most classification methods work well on the examples that were used in their establishment. The 16 patients test set was used to assess prediction accuracy. The cutoff for the classification was determined by using a ROC curve. With the selected cutoff, the numbers of correct prediction for relapse and survival patients in the test set were determined.

Overall prediction: Gene expression profiling of 63 Duke's B colon cancer patients led to identification of 4 genes that have differential expression (down regulation or turned off) in these patients. These genes are SEQ ID NO: 1, SEQ ID NO: 2, SEQ ID NO: 3, and SEQ ID NO: 4. Thirty-six of the patients have remained disease-free for more than 3 years while 27 patients had tumor relapse within 3 years. Using the 3 gene markers portfolio of SEQ ID NO: 2, SEQ ID NO: 3, and SEQ ID NO: 4, 22 of the 27 relapse patients and 27 of 36 disease-free patients are identified correctly. This result represents a sensitivity of 82% and a specificity of 75%. The positive predictive value is 71% and the negative predictive value is 84%.

### Example 4

Further Sampling

Frozen tumor specimens from 74 coded Dukes' B colon cancer patients were then studied. Primary tumor and adjacent non-neoplastic colon tissue were collected at the time of surgery. The histopathology of each specimen was reviewed to confirm diagnosis and uniform involvement with tumor. Regions chosen for analysis contained a tumor cellularity greater than 50% with no mixed histology. Uniform follow-up information was also available.

### Example 5

Gene Expression Analysis

Total RNA was extracted from the samples of Example 4 according to the method described in Examples 1-3. Arrays were scanned using standard Affymetrix protocols and scanners. For subsequent analysis, each probe set was considered as a separate gene. Expression values for each gene were calculated by using Affymetrix GeneChip analysis software MAS 5.0. All data used for subsequent analysis passed quality control criteria.

Statistical Methods

Gene expression data were first subjected to a variation filter that excluded genes called “absent” in all the samples. Of the 22,000 genes considered, 17,616 passed this filter and were used for clustering. Prior to the hierarchical clustering, each gene was divided by its median expression level in the patients. Genes that showed greater than 4-fold changes over the mean expression level in at least 10% of the patients were included in the clustering. To identify patient subgroups with distinct genetic profiles, average linkage hierarchical clustering and k-mean clustering was performed by using GeneSpring 5.0 (San Jose, Calif.) and Partek 5.1 software (St. Louis, Mo.), respectively. T-tests with Bonferroni corrections were used to identify genes that have different expression levels between 2 patient subgroups implicated by the clustering result. A Bonferroni corrected P value of 0.01 was chosen as the threshold for gene selection. Patients in each cluster that had a distinct expression profile were further examined with the outcome information.

In order to identify gene markers that can discriminate the relapse and the disease-free patients, each subgroup of the patients was analyzed separately as described further below. All the statistical analyses were performed using S-Plus software (Insightful, Va.).

Patient and Tumor Characteristics

Clinical and pathological features of the patients and their tumors are summarized in Table 1. The patients had information on age, gender, TNM stage, grade, tumor size and tumor location. Seventy-three of the 74 patients had data on the number of lymph nodes that were examined, and 72 of the 74 patients had estimated tumor size information. The patient and tumor characteristics did not differ significantly between the relapse and non-relapse patients. None of the patients received pre-operative treatment. A minimum of 3 years of follow-up data was available for all the patients in the study.

Patient Subgroups Identified by Genetic Profiles

Unsupervised hierarchical clustering analysis resulted in a cluster of the 74 patients on the basis of the similarities of their expression profiles measured over 17,000 significant genes. Two subgroups of patients were identified that have over 600 differentially expressed genes between them (p<0.00001). The larger subgroup and the smaller subgroup contained 54 and 20 patients, respectively. In the larger subgroup of the 54 patients only 18 patients (33%) developed tumor relapse within 3 years whereas in the smaller subgroup of the 20 patients 13 patients (65%) had progressive diseases. Chi square analysis gave a p value of 0.028.

Two dominant gene clusters that had drastic differential expression between the two types of tumors were selected and examined. The first gene cluster had a group of down-regulated genes in the smaller subgroup of the 20 patients, represented by liver-intestine specific cadherin 17, fatty acid binding protein 1, caudal type homeo box transcription factors CDX1 and CDX2, mucin and cadherin-like protein MUCDHL. The second gene cluster is represented by a group of up-regulated genes in the smaller subgroup including serum-inducible kinase SNK, annexin A1, B cell RAG associated protein, calbindin 2, and tumor antigen L6. The smaller subgroup of the 20 patients thus represent less differentiated tumors on the basis of their genetic profiles.

Gene Signature and its Prognostic Value

In order to identify gene markers that can discriminate the relapse and the disease-free patients, each subgroup of the patients were analyzed separately. The patients in each subgroup were first divided into a training set and a testing set with approximately equal number of patients. The training set was used to select the gene markers and to build a prognostic signature. The testing set was used for independent validation. In the larger subgroup of the 54 tumors, 36 patients had remained disease-free for at least 3 years after their initial diagnosis and 18 patients had developed tumor relapse with 3 years. The 54 patients were divided into two groups. The training set contained 21 disease-free patients and 6 relapse patients. In the smaller subgroup of the 20 tumors, 7 patients had remained disease-free for at least 3 years and 13 patients had developed tumor relapse with 3 years. The 20 patients were divided into two groups. The training set contained 4 disease-free patients and 7 relapse patients. To identify a gene signature that discriminates the good prognosis group from the poor prognosis group, a supervised classification method was used on each of the training sets. Univariate Cox proportional hazards regression was used to identify genes whose expression levels are correlated to patient survival time. Genes were selected using p-values less than 0.02 as the selection criteria. Next, t-tests were performed on the selected genes to determine the significance of the differential expression between relapse and disease-free patients (P<0.01). To avoid selection of genes that over-fit the training set, re-sampling of 100 times was performed with the t-test in order to search for genes that have significant p values in more than 80% of the re-sampling tests. Seven genes (Table 2) were selected from the 27 patient training set and 15 genes (Table 3) were selected from the 11 patient training set. Taking the 22 genes and cadherin 17 together, a Cox model to predict patient recurrence was built using the S-Plus software. The Kaplan-Meier survival analysis showed a clear difference in the probability that patients would remain disease free between the group predicted with good prognosis and the group predicted with poor prognosis (FIG. 3).

Several genes are related to cell proliferation or tumor progression. For example, tyrosine 3 monooxygenase tryptophan 5-monooxygenase activation protein (YWHAH) belongs to 14-3-3 family of proteins that is responsible for G2 cell cycle control in response to DNA damage in human cells. RCC1 is another cell cycle gene involved in the regulation of onset of chromosome condensation. BTEB2 is a zinc finger transcription factor that has been implicated as a beta-catenin independent Wnt-1 responsive genes. A few genes are likely involved in local immune responses. Immunoglobulin-like transcript 5 protein is a common inhibitory receptor for MHC I molecules. A unique member of the gelsolin/villin family capping protein, CAPG is primarily expressed in macrophages. LAT is a highly tyrosine phosphorylated protein that links T cell receptor to cellular activation. Thus both tumor cell- and immune cell-expressed genes can be used as prognostic factors for patient recurrence.

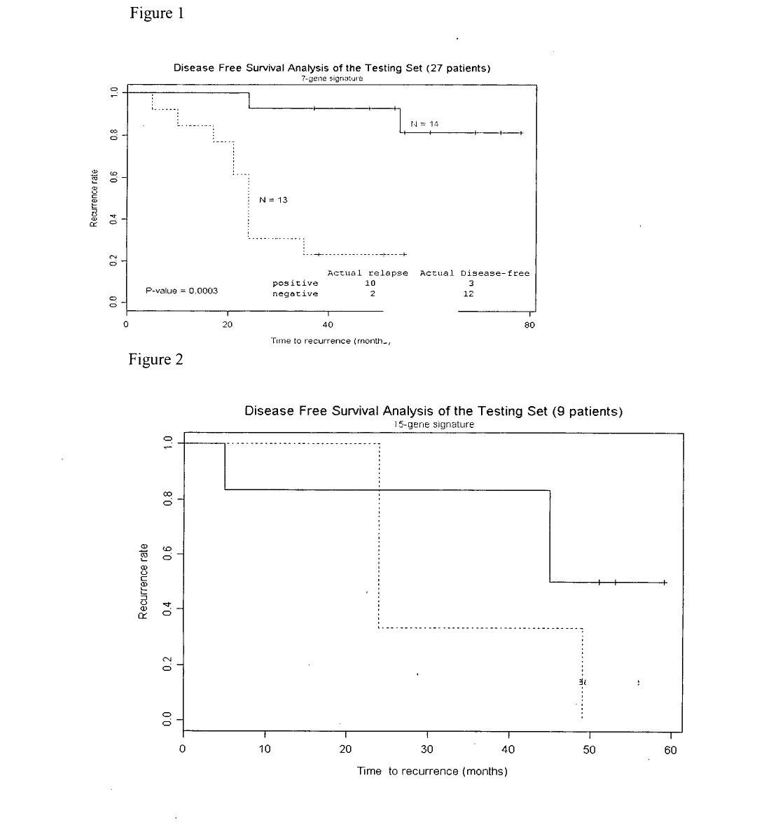

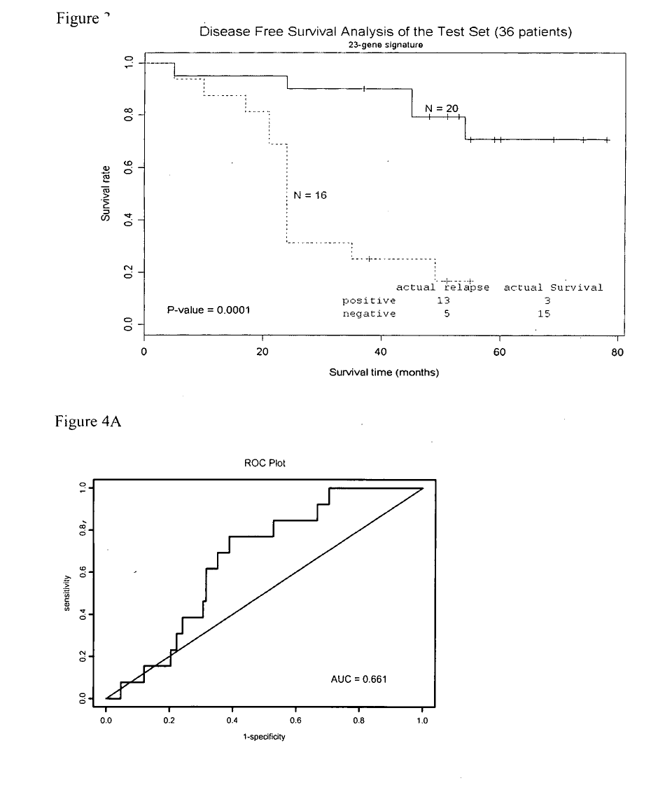

In order to validate the 23-gene prognostic signature, the patients in the two testing sets that included 27 patients from the larger subgroup and 9 patients from the smaller subgroup were combined and outcome was predicted for the 36 independent patients in the testing sets. This testing set consisted of 18 patients who developed tumor relapses within 3 years and 18 patients who had remained disease free for more than 3 years. The prediction resulted in 13 correct relapse classification and 15 correct disease-free classifications. The overall performance accuracy was 78% (28 of 36) with a sensitivity of 72% (13 of 18) and a specificity of 83% (15 of 18). This performance indicates that the Dukes' B patients that have a value below the threshold of the prognostic signature have a 13-fold odds ratio of (95% CI: 2.6, 65; p=0.003) developing a tumor relapse within 3 years compared with those that have a value above the threshold of the prognostic signature. Furthermore, the Kaplan-Meier survival analysis showed a significant difference in the probability that patients would remain disease free between the group predicted with good prognosis and the group predicted with poor prognosis (P<0.0001). In a multivariate Cox proportional hazards regression, the estimated hazards ratio for tumor recurrence was 0.41 (95% confidence interval, 0.24 to 0.71; P=0.001), indicating that the 23-gene set represents a prognosis signature 5 and it is inversely associated with a higher risk of tumor recurrence. Using the seven gene portfolio (Table 2), an 83% sensitivity and 80% specificity were obtained (based on a 12 relapse and 15 survivor sample set). Using the 15 gene portfolio (Table 3), a 50% sensitivity and 100% specificity were obtained (based on 6 relapse and three survivor sample sets). FIGS. 1 and 2 are graphical portrayals of the Kaplan-Meier analyses for the seven and fifteen gene portfolios respectively.

Furthermore, as these results demonstrate, prognosis can be derived from gene expression profiles of the primary tumor.

TABLE 1
Clinical and Pathological Characteristics of Patients and Their Tumors

| | Disease-free | | Recurrence | | |
|---|---|---|---|---|---|
| Characteristics | no. of patients (%) | | | | P Value* |
| Age | 43 | | 31 | | 0.7649 |
| Mean | 58.93 | | 58.06 | | |
| Sex | 43 | | 31 | | 0.8778 |
| Female | 23 | (53) | 18 | (58) | |
| Male | 20 | (47) | 13 | (42) | |
| T Stage | 43 | | 31 | | 0.2035 |
| 2 | 12 | (28) | 5 | (16) | |
| 3 | 29 | (67) | 26 | (84) | |
| 4 | 2 | (5) | 0 | (0) | |
| Differentiation | 43 | | 31 | | 0.4082 |
| Poor | 5 | (12) | 6 | (19) | |
| Moderate | 37 | (86) | 23 | (74) | |
| Well | 1 | (2) | 2 | (6) | |
| Tumor size | 41 | | 31 | | 0.1575 |
| <5 | 29 | (71) | 16 | (52) | |
| >=5 | 12 | (29) | 15 | (48) | |
| Location | 43 | | 31 | | 0.7997 |
| LC | 1 | (2) | 1 | (3) | |
| RC | 17 | (40) | 10 | (32) | |
| TC | 6 | (14) | 3 | (10) | |
| SC | 19 | (44) | 17 | (55) | |
| Number of LN examined | 43 | | 30 | | 0.0456 |
| Mean | 12.81 | | 8.63 | | |

*P values for Age, Lymph node number and Tumor content are obtained by t tests; P values for others are obtained by χ² tests.

TABLE 2
7 Gene List

| Accession | SEQ ID NO: |
|---|---|
| AF009643.1 | 7 |
| NM_003405.1 | 8 |
| X06130.1 | 9 |
| AB030824.1 | 10 |
| NM_001747.1 | 11 |
| AF036906.1 | 12 |
| BC005286.1 | 13 |

TABLE 3
15 Gene List

| | SEQ ID |
|---|---|
| Accession | NO: |
| NM_012345.1 | 14 |
| NM_030955.1 | 15 |
| NM_001474.1 | 16 |
| AF239764.1 | 17 |
| D13368.1 | 18 |
| NM_012387.1 | 19 |
| NM_016611.1 | 20 |
| NM_014792.1 | 21 |
| NM_017937.1 | 22 |
| NM_001645.2 | 23 |
| AL545035 | 24 |
| NM_022078.1 | 25 |
| AL133089.1 | 26 |
| NM_001271.1 | 27 |
| AL137428.1 | 28 |

TABLE 4
Twenty-three genes form the prognostic signature.

| | P value | |
|---|---|---|
| SEQ ID NO: | (Cox) | Gene Description |
| 7 | 0.0011 | immunoglobulin-like transcript 5 protein |
| 8 | 0.0016 | tyrosine 3-monooxygenasetryptophan |
| | | 5-monooxygenase activation protein |
| 9 | 0.0024 | cell cycle gene RCC1 |
| 10 | 0.0027 | transcription factor BTEB2 |
| 11 | 0.0045 | capping protein (actin filament), gelsolin- |
| | | like (CAPG) |
| 12 | 0.0012 | linker for activation of T cells (LAT) |
| 13 | 0.0046 | Lafora disease (laforin) |
| 14 | 0.0110 | nuclear fragile X mental retardation protein |
| | | interacting protein 1 (NUFIP1) |
| 15 | 0.0126 | disintegrin-like and metalloprotease |
| | | (reprolysin type) with thrombospondin |
| | | type 1 motif, 12 (ADAMTS12) |
| 16 | 0.0126 | G antigen 4 (GAGE4) |
| 17 | 0.0130 | EGF-like module-containing mucin-like |
| | | receptor EMR3 |
| 18 | 0.0131 | alanine:glyoxylate aminotransferase |
| 19 | 0.0131 | peptidyl arginine deiminase, type V (PAD) |
| 20 | 0.0136 | potassium inwardly-rectifying channel, |
| | | subfamily K, member 4 (KCNK4) |
| 21 | 0.0139 | KIAA0125 gene product (KIAA0125) |
| 22 | 0.0142 | hypothetical protein FLJ20712 (FLJ20712) |
| 23 | 0.0145 | apolipoprotein C-I (APOC1) |
| 24 | 0.0146 | Consensus includes gb:AL545035 |
| 25 | 0.0149 | hypothetical protein FLJ12455 (FLJ12455) |
| 26 | 0.0150 | Consensus includes gb:AL133089.1 |
| 27 | 0.0151 | chromodomain helicase DNA binding |
| | | protein 2 (CHD2) |
| 28 | 0.0152 | Consensus includes gb:AL137428.1 |
| 6 | Not tested | Cadherin 17 |

### Example 6

In this study we now have completed an independent assessment of this prognostic signature in an independent series of 123 Dukes' B colon cancer patients obtained from two sources. In addition, we developed a RTQ-PCR assay in order to test the prognostic gene signature in FPE samples. Our data provide validation with high confidence of a pre-specified prognostic gene signature for Dukes' B colon cancer patients.

Purpose: The 5 year survival rate for patients with Dukes' B colon cancer is approximately 75%. In our earlier genome-wide measurements of gene expression we identified a 23-gene signature that sub-classifies patients with Dukes' B according to clinical outcome and may provide a better predictor of individual risk for these patients. Wang, et al. (2005). The present study validates this gene signature in an independent and more diverse group of patients, and develops this prognostic signature into a clinically-feasible test using fixed paraffin-embedded (FPE) tumor tissues.

Patients and Methods: Using Affymetrix U133a GeneChip we analyzed the expression of the 23 genes in total RNA of frozen tumor samples from 123 Dukes' B patients who did not receive adjuvant systemic treatment. Furthermore, we developed a real time quantitative (RTQ)-PCR assay for this gene signature in order to perform the test with standard clinical FPE samples.

Results: In the independent validation set of 123 patients, the 23-gene signature proved to be highly informative in identifying patients who would develop distant metastasis (hazard ratio, HR 2.56; 95% confidence interval CI, 1.01-6.48), even when corrected for the traditional prognostic factors in multivariate analysis (HR, 2.73; 95% CT, 0.97-7.73). The RTQ-PCR assay developed for this gene signature was also validated in an independent set of 110 patients with available FPE tissue and was a strong prognostic factor for the development of distant recurrence (HR, 6.55; 95% CI, 2.89-14.8) in both univariate and multivariate analyses (HR, 13.9; 95% CI, 5.22-37.2).

Conclusion: Our results validate the pre-defined prognostic gene signature for Dukes' B colon cancer patients in an independent population and show the feasibility of testing the gene signature using RTQ-PCR on standard FPE specimens. The ability of such a test to identify colon cancer patients that have an unfavorable outcome demonstrates a clinical relevance to help identify patients at high risk for recurrence who require more aggressive therapeutic options.

Patients and Methods

Patient Samples

Frozen tumor specimens from 123 coded Dukes' B colon cancer patients and FPE tumor specimens from 110 of these patients were obtained from Cleveland Clinic Foundation (Cleveland, Ohio), Aros Applied Biotechnology, LLC (Aarhus, Denmark) and Proteogenix, LLC (Culver City, Calif.) according to the Institutional Review Board approved protocols at individual sites. Fifty-four patients have matched frozen and FPE samples. Archived primary tumor samples were collected at the time of surgery. The histopathology of each specimen was reviewed to confirm diagnosis and tumor content. The total cell population was composed of at least 70% tumor cells.

At least 3 years of follow-up were required, except for patients who developed distant relapse before that time. The patients were treated by surgery only. Post-surgery patient surveillance was carried out according to general practice for colon cancer patients including physical exam, blood counts, liver function tests, serum CEA, and colonoscopy for the patients. Selected patients had abdominal CT scan and chest X-ray. If tumor relapse was suspected, the patient underwent intensive work-tip including abdominal/pelvic CT scan, chest X-ray, colonoscopy and biopsy when applicable. Time to recurrence or disease-free time was defined as the time period from the date of surgery to confirmed tumor relapse date for relapsed patients and from the date of surgery to the date of last follow-up for disease-free patients.

Microarray Analysis

All tumor tissues were processed for RNA isolation as described in our initial study. Examples above and Wang et al. (2005). Biotinylated targets were prepared using published methods (Affymetrix, Santa Clara, Calif.) (Lipshutz et al. (1999)) and hybridized to Affymetrix U133a GeneChips (Affymetrix, Santa Clara, Calif.). Arrays were scanned using the standard Affymetrix protocol. Each probe set was considered a separate gene. Expression values for each gene were calculated using Affymetrix GeneChip® analysis software MAS 5.0 and according to the analysis method described previously. Wang et al. (2005)

RNA Isolation from FPE samples.

FPE tissue was available for 110 patients. The FPE samples were either formalin-fixed (n=45) or Hollandes-fixed (n=65) FPE tissues. RNA isolation from FPE tissue samples was carried out according to a modified protocol using High Pure RNA Paraffin Kit (Roche Applied Sciences, Indianapolis, Ind.). FPE tissue blocks were sectioned depending on the size of the blocks (6-8 mm=6×10 μm, 8-≧10 mm=3×10 μm). Sections were de-paraffinized as described in the manufacturer's manual. The tissue pellet was dried in oven at 55° C. for 10 minutes and resuspended in 100 μL of tissue lysis buffer, 16 μL 10% SDS and 80 μL Proteinase K. The sample was vortexed and incubated in a thermomixer set at 400 rpm for 3 hours at 55° C. Subsequent steps of sample processing were performed according the Kit manual. The RNA sample was quantified by OD 260/280 readings using spectrophotometer and diluted to a final concentration of 50 ng/μL. The isolated RNA samples were stored in RNase-free water at −80° C. until use.

RTQ-PCR Analysis

Seven genes of the 23-gene signature were evaluated using a one-step multiplex RTQ-PCR assay with the RNA samples isolated from FPE tissues. In order to minimize the variability of the RTQ-PCR reaction, four housekeeping control genes including β-actin, HMBS, GUSB, and RPL13A, were used to normalize the input quantity of RNA. To prevent any contaminating DNA in the samples from amplification, PCR primers or probes for RTQ-PCR assay were designed to span an intron so that the assay would not amplify any residual genomic DNA. One-hundred nanograms of total RNA were used for the one-step RTQ-PCR reaction. The reverse transcription was carried out using 40×Multiscribe and RNase inhibitor mix contained in the TaqMan® one-step PCR Master Mix reagents kit (Applied Biosystems, Fresno, Calif.). The cDNA was then subjected to the 2×Master Mix without uracil-N-glycosylase (UNG). PCR amplification was performed on the ABI 7900HT sequence detection system (Applied Biosystems, Frenso, Calif.) using the 384-well block format with 10 μL reaction volume. The concentrations of the primers and the probes were 4 and 2.5 μmol/L, respectively.

The reaction mixture was incubated at 48° C. for 30 minutes for the reverse transcription, followed by an Amplitaq® activation step at 95° C. for 10 minutes and then 40 cycles of 95° C. for 15 seconds for denaturing and of 60° C. for 1 minute for annealing and extension. A standard curve was generated from a range of 100 pg to 100 ng of the starting materials, and when the R² value was >0.99, the cycle threshold (Ct) values were accepted. In addition, all primers and probes were optimized towards the same amplification efficiency according to the manufacturer's protocol. We used Applied Biosystems' Assay-On-Demand for 4 of the 7 genes (BTEB2, LAT, CAPG, and Immunoglobulin-like transcript 5 protein). Sequences of the primers and probes for the other 3 genes and the 4 housekeeping control genes were as follows, each written in the 5′ to 3′ direction:

SEQ ID NO: 29

| | |
|---|---|
| Laforin forward, | CATTATTCAAGGCCGAGTACAGATG; |

SEQ ID NO: 30

| | |
|---|---|
| Laforin reverse, | CACGTACACGATGTGTCCCTTCT; |

SEQ ID NO: 31

| | |
|---|---|
| Laforin probe, | CAGGCGGTGTGCCTGCTGCAT. |

SEQ ID NO: 32

| | |
|---|---|
| RCC1 forward, | TTTGTGGTGCCTATTTCACCTTT; |

SEQ ID NO: 33

| | |
|---|---|
| RCC1 reverse, | CGGAGTTCCAAGCTGATGGTA; |

SEQ ID NO: 34

| | |
|---|---|
| RCC1 probe, | CCACGTGTACGGCTTCGGCCTC. |

SEQ ID NO: 35

| | |
|---|---|
| YWHAH forward, | GGCGGAGCGCTACGA; |

SEQ ID NO: 36

| | |
|---|---|
| YWHAH reverse, | TTCATTCGAGAGAGGTTCATTCAG; |

SEQ ID NO: 37

| | |
|---|---|
| YWHAH probe, | CCTCCGCTATGAAGGCGGTGÃ |

SEQ ID NO: 38

| | |
|---|---|
| β-actin forward, | AAGCCACCCCACTTCTCTCTAA; |

SEQ ID NO: 39

| | |
|---|---|
| β-actin reverse, | AATGCTATCACCTCCCCTGTGT; |

SEQ ID NO: 40

| | |
|---|---|
| β-actin probe, | AGAATGGCCCAGTCCTCTCCCAAGTC. |

SEQ ID NO: 41

| | |
|---|---|
| HMBS forward, | CCTGCCCACTGTGCTTCCT; |

SEQ ID NO: 42

| | |
|---|---|
| HMBS reverse, | GGTTTTCCCGCTTGCAGAT; |

SEQ ID NO: 43

| | |
|---|---|
| HMBS probe, | CTGGCTTCACCATCG. |

SEQ ID NO: 44

| | |
|---|---|
| GUSB forward, | TGGTTGGAGAGCTCATTTGGA; |

SEQ ID NO: 45

| | |
|---|---|
| GUSB reverse, | ACTCTCGTCGGTGACTGTTCAG; |

SEQ ID NO: 46

| | |
|---|---|
| GUSB probe, | TTTTGCCGATTTCATG. |

SEQ ID NO: 47

| | |
|---|---|
| RPL13A forward, | CGGAAGAAGAAACAGCTCATGA; |

SEQ ID NO: 48

| | |
|---|---|
| RPL13A reverse, | CCTCTGTGTATTTGTCAATTTTCTTCTC; |

SEQ ID NO: 49

| | |
|---|---|
| RPL13A probe, | CGGAAACAGGCCGAGAA. |

For each sample ΔCt=Ct (target gene)−Ct (average of four control genes) was calculated. ΔCt normalization has been widely used in clinical RTQ-PCR assay.

Statistical Methods

The data variability resulting from different protocols for sample handling at individual clinical institutions were minimized by using analysis of variance (ANOVA) on the gene expression data. Cadherin 17 gene expression measurement on the array was used to determine the assignment of the patient into the subgroups as described in our previous study. Above examples and Wang et al. (2005). Patients with detectable Cadherin 17 expression levels were classified as subgroup I and their outcome was predicted using the 7-gene subset of the 23-gene signature. Patients with undetectable Cadherin 17 expression levels were classified as subgroup II and their outcome was predicted using the 15-gene subset of the 23-gene signature. The relapse score was calculated for each patient and used to classify the patient into high or low risk groups for developing distant metastasis within 3 years. Patients with a relapse score >0 were classified as high risk and patients with a relapse score <0 were called as low risk. The calculation of the relapse score was as follows:

$$Relapse Hazard Score = A \cdot I + {\sum }_{i = 1}^{7} I \cdot {w}_{i} {x}_{i} + B \cdot ( 1 - I ) + {\sum }_{j = 1}^{15} ( 1 - I ) \cdot {w}_{j} {x}_{j}$$

$$where$$

$$I = \{ \begin{matrix}1 & if Cadherin 17 expression is detected \\ 0 & if Cadherin 17 expression is undetected\end{matrix}$$

A and B are constants
wᵢ is the standardized Cox regression coefficient
xᵢ is the expression value in log2 scale

Kaplan-Meier survival plots (Kaplan et al. (1958)) and log-rank tests were used to assess the difference of the predicted high and low risk groups. Sensitivity was defined as the percent of the patients with distant metastasis within 3 years that were predicted correctly by the gene signature, and specificity was defined as the percent of the patients free of distant recurrence for at least 3 years that were predicted as being free of recurrence by the gene signature. Odds ratio (OR) was calculated as the ratio of the odds of distant metastasis between the predicted relapse patients and relapse-free patients. Univariate and multivariate analyses using the Cox proportional hazard regression were performed on the individual clinical parameters of patients and the combination of the clinical parameters and the gene signature, including age, gender, T stage, grade and tumor size. The HR and its 95% CI were derived from these results. All statistical analyses were performed using S-Plus® 6 1 software (Insightful, Fairfax Station, Va.).

Results

Patient and Tumor Characteristics

Clinical and pathological features of the patients and their tumors are summarized in Table 5 and Table 6. All patients had information on age, gender, TNM stage, grade, tumor size and tumor location. The patient and tumor characteristics did not differ significantly between the relapse and non-relapse patients. The patients were treated by surgery only and none of the patients received neo-adjuvant or adjuvant treatment. A minimum of 3 years of follow-up data was available for all the patients in the study with the exception of those with relapse <3 years.

TABLE 5
Patient and tumor characteristics (frozen tumor tissue study)

| | Factor | | | | | |
|---|---|---|---|---|---|---|
| | | | | | AROS + | |
| | AROS | | CCF | | CCF | |
| | Number | % | Number | % | Number | % |
| Age | 67 years | | 70 years | | 69 years | |
| Sex | | | | | | |
| Male | 26 | (53) | 37 | (50) | 63 | (51) |
| Female | 23 | (47) | 37 | (50) | 60 | (49) |
| T Stage | | | | | | |
| T2 | 0 | | 0 | | 0 | |
| T3 | 37 | (76) | 64 | (86) | 101 | (82) |
| T4 | 7 | (14) | 10 | (14) | 17 | (14) |
| Unknown | 5 | (10) | 0 | | 5 | (4) |
| Grade | | | | | | |
| Good | 9 | (19) | 6 | (8) | 15 | (12) |
| Moderate | 32 | (65) | 56 | (76) | 88 | (72) |
| Poor | 8 | (16) | 12 | (16) | 20 | (16) |
| Metastasis < 3 yr | | | | | | |
| Yes | 9 | (18) | 4 | (5) | 13 | (11) |
| No | 40 | (82) | 68 | (92) | 108 | (88) |
| Censored | 0 | | 2 | (3) | 2 | (1) |

TABLE 6
Patient and tumor characteristics (FPE study)

| | Factor | | | | | |
|---|---|---|---|---|---|---|
| | | | | | Proteogenex + | |
| | Proteogenex | | CCF | | CCF | |
| | Number | % | Number | % | Number | % |
| Age | 66 years | | 71 years | | 69 years | |
| Sex | | | | | | |
| Male | 13 | (32) | 36 | (52) | 49 | (45) |
| Female | 28 | (68) | 33 | (48) | 61 | (55) |
| T Stage | | | | | | |
| T2 | 2 | (5) | 0 | | 2 | (2) |
| T3 | 31 | (76) | 60 | (87) | 91 | (83) |
| T4 | 8 | (19) | 9 | (13) | 17 | (15) |
| Grade | | | | | | |
| Good | 4 | (10) | 6 | (9) | 10 | (9) |
| Moderate | 26 | (63) | 51 | (74) | 77 | (70) |
| Poor | 5 | (12) | 12 | (17) | 17 | (16) |
| Unknown | 6 | (15) | 0 | | 6 | (5) |
| Metastasis < 3 yr | | | | | | |
| Yes | 11 | (27) | 6 | (9) | 17 | (15) |
| No | 30 | (73) | 62 | (90) | 92 | (84) |
| Censored | 0 | | 1 | (1) | 1 | (1) |

Analysis of the Gene Signature in the Fresh Frozen Samples

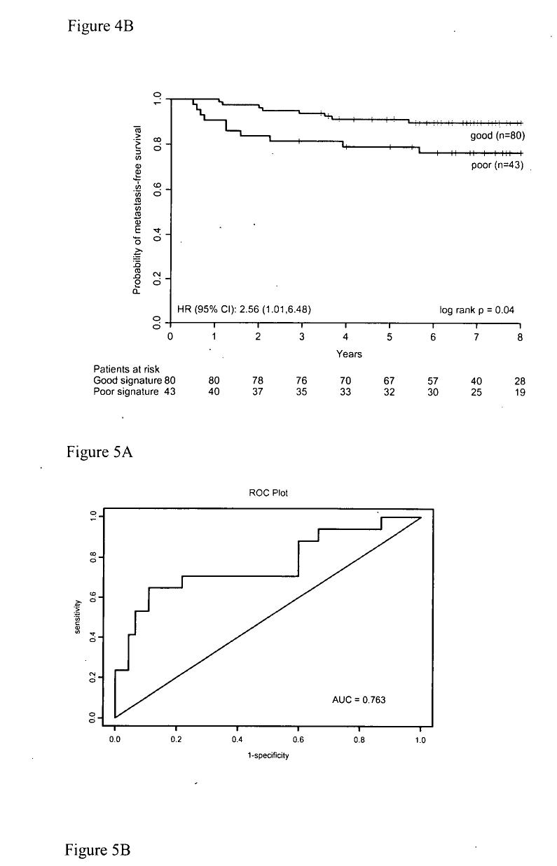

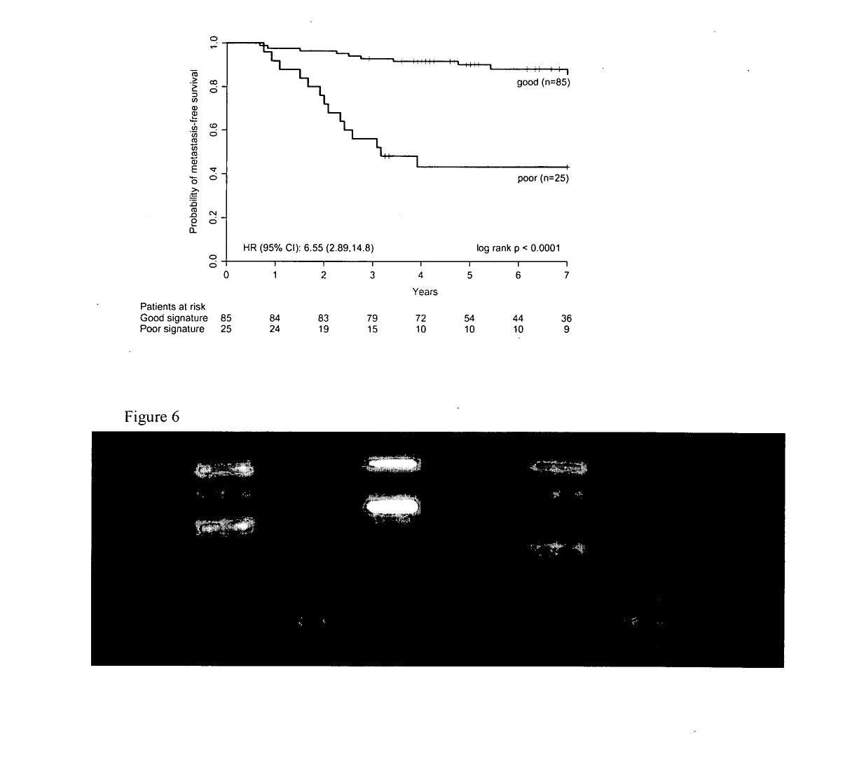

Survival analysis was performed as a function of the 23-gene signature. First, the ROC curve was evaluated (FIG. 4). The area under the curve (AUC) was used to assess the performance of a predictor. The 23-gene predictor gave an AUC value of 0.66. Using the 3-yr defining point, the relapse score calculated from this method correctly predicted 8 of the 13 relapses (62% sensitivity) that occurred within 3 years and 74 of the 108 non-relapsers (69% specificity). Although the frequency of tumor relapse was only 11% in this group of the 123 patients, the Kaplan-Meier analysis produced survival curves for the patient groups and the log rank test showed a significant difference in the time to recurrence between the group predicted with good prognosis and the group predicted with poor prognosis (P=0.04) (FIG. 4). In the univariate and multivariate analyses of the 123 patients, the 23-gene signature proved to be highly informative in identifying patients who would develop distant metastasis (hazard ratio, HR 2.56; 95% confidence interval CI, 1.01-6.48), even when corrected for the traditional prognostic factors in multivariate analysis (HR, 2.73; 95% CI, 0.97-7.73).

In the patient sample group of our initial-study (Wang et al. (2005)), we detected 2 subgroups of tumors representing well- and poorly-differentiated tumors, respectively. Cadherin 17 gene expression was used to stratify the Dukes' B tumors into the two subgroups and the prognostic gene signature was designed to include classifiers for subgroup I (7 genes) and subgroup II (15 genes). In the present validation study, we examined an independent sample group of 123 Dukes' B patients from 2 sources and found that subgroup II only accounted for a very small portion of a typical make-up of Dukes' B tumors (2%). Therefore, we simplified the prognostic gene signature by removing the 15 genes that were selected for subgroup II in the subsequent RTQ-PCR assay.

The microarray dataset has been submitted to the NCBI/Genbank GEO database (series entry pending).

Analysis of the Gene Signature in the FPE Samples RTQ-PCR assay was performed using the 7 genes that were selected for the subgroup I patients as mentioned above. These 7 genes should be able to classify the outcomes of greater than 95% of the patients in a representative population. Survival analysis was performed. First, the ROC curve was evaluated (FIG. 5). The parameter that was used to assess the performance of a predictor was the area under the curve (AUC). The 7-gene predictor gave an AUC value of 0.76. Using the 3-yr defining point, the relapse score calculated from this method correctly predicted 11 of the 17 relapses (65% sensitivity) that occurred within 3 years and 78 of the 92 non-relapsers (85% specificity). Furthermore, the Kaplan-Meier analysis and the log rank test both showed a significant difference in the time to recurrence between the group predicted with good prognosis and the group predicted with poor prognosis (P<0.0001) (FIG. 5). In the 110 patients, the 7-gene signature was confirmed as a strong prognostic factor for the development of distant recurrence (HR, 6.55; 95% CI, 2.89-14.8) and in both in univariate and in multivariate analyses (HR, 13.9; 95% CI, 5.22-37.2) (Table 7).

TABLE 7
Uni-and Multivariate analysis for DMFS
Multivariate & Univariate Cox Analysis of Distant Metastasis-Free
Survival in 132 ER positive Breast Cancer Patients

| | Univariate analysis | | | Multivariate analysis¹ | | |
|---|---|---|---|---|---|---|
| | HR² | (95% CI) | p value | HR | (95% CI) | p value |
| Age | 0.98 | (0.95-1.01) | 0.2420 | 0.97 | (0.94-1.01) | 0.1025 |
| Sex³ | 0.81 | (0.35-1.85) | 0.6129 | 1.15 | (0.44-3.01) | 0.7756 |
| T Stage | 0.70 | (0.22-2.28) | 0.5565 | 1.30 | (0.31-5.48) | 0.7248 |
| Grade⁴ | 1.17 | (0.35-3.95) | 0.8018 | 0.46 | (0.12-1.70) | 0.2420 |
| Tumor | 0.61 | (0.26- 1.40) | 0.2460 | 0.59 | (0.24-1.44) | 0.2440 |
| Size⁵ | | | | | | |
| 7-gene | 6.55 | (2.89-14.8) | 6.6E-06 | 13.94 | (5.22-37.2) | 1.5E-07 |
| Signature | | | | | | |

¹The multivariate model include 101 patients, due to missing values in 9 patients
²Hazard Ratio
³Sex: Male vs. Female
⁴Grade: Moderate & Well vs. Poor
⁵Tumor Size: >=5 mm vs. <5 mm

Among the common 54 patient samples used for both microarray-based assay and RTQ-PCR assay, the array results classified 15 patients as relapsers and 39 patients as non-relapsers while the RTQ-PCR results predicted 9 patients as relapsers and 45 patients as non-relapsers. Forty of the 54 patients (74%) were consistently predicted by both methods and 14 patients were predicted inconsistently between the methods (26%). Given that different types of tissue samples were used for the two assays (frozen vs FPE), the concordance in the classification results is high between the two methods. Among the 14 discordant samples, 4 patients had scores very close to the cutoffs (within 5% of the cutoffs) while the remaining 10 patients had very poorly correlated scores between the two methods (correlation coefficient: 0.15). We repeated the RTQ-PCR assay on the 10 discordant samples using the same RNA samples and the scores of the 2 RTQ-PCR assays gave a correlation coefficient of 0.998. The data suggested that the discordant scores of these patients might be due to differences in sampling of the same tumor. Further test is required in order to assess the variability of sampling in clinical FPE materials.

Discussion

We provide the results of a validation study on the 23-gene signature established previously. Above Examples and Wang et al. (2005). In the above study, the sensitivity and the specificity of the signature was 72% and 83%, respectively. This prognostic signature was used to predict distant recurrence in an independent series of 123 Dukes' B colon cancer patients according to the pre-specified criteria. Furthermore, we report the successful validation of distant recurrence in an independent set of 110 Dukes' B patients using a 7-gene signature using a RTQ-PCR assay of the FPE samples. This study brings us a step closer to the clinical application of such a molecular prognostic test for colon cancer patients. This highlights the efficacy of current treatment regimens for Dukes' B colon cancer patients.

In the patient sample group of our initial study (Wang et al. (2005)), unsupervised hierarchical clustering with over 17,000 informative genes detected 2 subgroups of tumors representing well-differentiated and less differentiated tumors, respectively. We used expression of the Cadherin 17 gene as an indicator to stratify the Dukes.' B tumors into the two subgroups and designed the prognostic gene signature to include classifiers for subgroup I (7 genes) and subgroup II (15 gene). The initial patient set may not have represented a typical make-up of the Dukes' B tumors, especially the ratio of the patients between the subgroup I and subgroup II. In the present validation study, we examined the independent sample groups from 2 sources and found that subgroup II only accounted for a very small portion of a typical make-up of Dukes' B tumors (2%) in the samples from both sites. Therefore, we simplified the prognostic gene signature by removing the 15 genes that were selected for subgroup II.

Studies that are aimed at developing molecular gene signatures must be rigorously validated and cannot be considered for clinical application until the results are properly confirmed and are demonstrated to be highly reproducible with regard to methodological, statistical and clinical aspects. In this respect, several criticisms have been raised concerning published gene-expression profiling studies on issues relating to the omission of independent validation sets, the sizes of training and testing sets, or possible confounding effects of treatment to the patient population studied. Ransohoff (2005); and Simon et al. (2003). Our present study represents the first successful validation of a pre-specified prognostic profile for colon cancer patients. The strength of the study relied on the diverse groups of patients from multiple institutions and the use of the standard clinical FPE materials. The tumor specimens were collected and stored according to institutional protocols, and the RNA samples were prepared using easily applicable procedures. Despite the differences in tissue handling at different institutions, the gene signature proved to be robust and produced results that were consistent with those from our initial analysis.

In conclusion, the results of the present validation study confirm the results of our initial report. The proven reproducibility of the results indicates that the prognostic gene signature can be recommended for future clinical studies and potentially for use in clinical practice. As approximately 20-30% of Dukes' B colon cancer patients relapse, the prognosis signature provides a powerful tool to select patients at high risk for relapse and possible additional adjuvant treatment. Liefers et al. (1998); and Markowitz et al. (2002). This ability to identify the patients that need intensive clinical intervention may lead to an improvement in disease survival.

### Example 7

Cepheid PCR Reactions

Materials & Methods

RNA Isolation from FFPE samples. RNA isolation from paraffin tissue sections was based on the methods and reagents described in the High Pure RNA Paraffin Kit manual (Roche) with the following modifications. 12×10 μm sections were taken from each paraffin embedded tissue samples. Sections were deparaffinized as described by Kit manual, the tissue pellet was dried in a 55° C. oven for 5-10 minutes and resuspended in 100 μl of tissue lysis buffer, 16 μl 10% SDS and 80 μl Proteinase K. Samples were vortexed and incubated in a thermomixer set at 400 rpm for 3 hours at 55° C. Subsequent sample processing was performed according High Pure RNA Paraffin Kit manual. Samples were quantified by OD 260/280 readings obtained by a spectrophotometer and the isolated RNA was stored in RNase-free water at −80° C. until use.

One-step Quantitative Real-Time Polymerase Chain Reaction. Appropriate mRNA reference sequence accession numbers in conjunction with Primer Express 2.0 were used to develop our hydrolysis probe Colon prognostic assays immunoglobulin-like transcript 5 protein (LILRB3), tyrosine 3-monooxygenasetryptophan 5-monooxygenase activation protein (YWHAH), cell cycle gene RCC1 (CHC1), transcription factor BTEB2 (KLF5), capping protein (actin filament) gelsolin-like (CAPG), linker for activation of T cells (LAT), lafora disease (EP2MA), ribosomal protein L13a (RPL13A), actin, beta actin (ACTB) and hydroxymethylbilane synthase (PBGD). Gene specific primers and hydrolysis probes for the optimized one-step qRT-PCR assay are listed in Table 8. Genomic DNA amplification was excluded by designing our assays around exon-intron splicing sites. Hydrolysis probes were labeled at the 5′ nucleotide with either FAM, Quasar 570, Texas Red or Quasar 670 as the reporter dye and at 3′ nucleotide with BHQ as the internal quenching dye.

Quantitation of gene-specific RNA was carried out in a 25 μl reaction tube on the Smartcycler II sequence detection system (Cepheid). For each assay gene standard curves were amplified before the genes were multiplexed in order to prove PCR efficiency. Standard curves for our markers consisted of target gene in total RNA samples that were at a concentration of 2×10², 1×10² and 5×10 ng per reaction. No target controls were also included in each assay run to ensure a lack of environmental contamination. All samples and controls were run in duplicate. Quantitative Real-Time PCR was carried out in a 25 μl reaction mix containing: 100 ng template RNA, RT-PCR Buffer (125 mM Bicine, 48 mM KOH, 287.5 nM KAc, 15% glycerol, 3.125 mM MgCl, 7.5 mM MnSO₄, 0.5 mM each of dCTP, dATP, dGTP and dTTP), Additives (125 mM Tris-Cl pH 8, 0.5mg/ml Albumin Bovine, 374.5 mM Trehalose, 0.5% Tween 20), Enzyme Mix (0.65 U Tth (Roche), 0.13 mg/ml Ab TP6-25, Tris-Cl 9 mM, Glycerol 3.5%), primer and probe concentrations were varied and are located in Table 9. Reactions were run on a Smartcycler II Sequence Detection System (Cepheid, Sunnyvale, Calif.). The following cycling parameters were followed: 1 cycle at 95° C. for 15 seconds; 1 cycle at 55° C. for 6 minutes; 1 cycle at 59° C. for 6 minutes; 1 cycle at 64° C. for 10 minutes and 40 cycles of 95° C. for 20 seconds, 58° C. for 30 seconds. After the PCR reaction was completed the Cepheid software and calculated Ct values were exported to Microsoft Excel.

TABLE 8
Colon Prognostic Primers and probe Sequences for Cepheid reactions

SEQ ID NO

| | | | |
|---|---|---|---|
| Forward Primer | EP2MA-462 | CATTATTCAAGGCCGAGTACAGATG | 9 |
| Reverse Primer | EP2MA-546 | CACGTACACGATGTGTCCCTTCT | 30 |
| Probe (5′TxR/3′BHQ) | EP2MA-493 | CAGGCGGTGTGCCTGCTGCAT-BHQ-TT | 31 |
| Forward Primer | CHC1-1023 | TTTGTGGTGCCTATTTCACCTTT | 32 |
| Reverse Primer | CHC1-1111 | CGGAGTTCCAAGCTGATGGTA | 33 |
| Probe (5′TxR/3′BHQ) | CHC1-1063 | CCACGTGTACGGCTTCG-BHQ-GCCTC | 34 |
| Forward Primer | YWHAH-245 | GGCGGAGCGCTAGGA | 35 |
| Reverse Primer | YWHAH0-317 | TTCATTCGAGAGAGGTTCATTCAG | 36 |
| Probe (5′FAM/3′BHQ) | YWHAH-268 | gCCTCCGGTATGAAGGC-BHQ-GGTGA | 37 |
| Forward Primer | B-actin-1030 | CCTGGCACCCAGCACAAT | 50 |
| Reverse Primer | B-actin-1099 | GCCGATCCACACGGAGTACTT | 51 |
| Probe (5′Cy3/3′BHQ) | B-actin-1052 | ATCAAGATCATTGCTCCTCC-BHQ2-TGAGCGC | 52 |
| Forward Primer | PBGD-131 | GCCTACTTTCCAAGCGGAGCCA | 53 |
| Reverse Primer | PBGD-213 | TTGCGGGTACCCACGCGAA | 54 |
| Probe (5′Cy5/3′BHQ) | PBGD-161 | AACGGCAATGCGGCTGCAACGGCGGAA-BHQ2-TT | 55 |
| Forward Primer | RPL13A-527 | CGGAAGAAGAAACAGCTCATGA | 47 |
| Reverse Primer | RPL13A-605 | CCTCTGTCTATTTGTCAATTTTCTTCTC | 48 |
| Probe (5′Cy3/3′BHQ) | RPL13A-554 | CGGAAAGAGGCCGAGAA-BHQ-TT | 49 |
| Forward Primer | KLF5-1374 | CAACCTGTCAGATACAATAGAAGGAGTAA | 56 |
| Reverse Primer | KLF5-1451 | GCAACCAGGGTAATCGCAGTA | 57 |
| Probe (5′FAM/3′BHQ) | KLF5-1404 | gCCCGATTTGGAGAAACGACGCATC-BHQ1-TT | 58 |
| Forward Primer | CAPG-1009 | GCAGTACGCCCCGAACACT | 59 |
| Reverse Primer | CAPG-1079 | AAAATTGCTTGAAGATGGGACTCT | 60 |
| Probe (5′TxR/3′BHQ) | CAPG-1032 | TGGAGATTCTGCCTCAG-BHQ2-GGCCGT | 61 |
| Forward Primer | LILRB3-1287 | CCCTGGAACTCATGGTCTCA | 62 |
| Reverse Primer | LILRB3-1396 | CGAGACCCCAATCAAAACCT | 63 |
| Probe (5′FAM/3′BHQ) | LILRB3-1338 | CAGGGCCGCCCTCCACACCTG-BHQ1-TT | 64 |
| Forward Primer | LAT-625 | CCACCGGACGCCATC | 65 |
| Reverse Primer | LAT-687 | TTCTCGTAGCTCGCCACACT | 66 |
| Probe (5′Cy3/3′BHQ) | LAT-641 | TCCCGGCGGGATTCTGATG-BHQ1-TT | 67 |

TABLE 9
Colon Prognostic Primer and Probe Concentrations

```

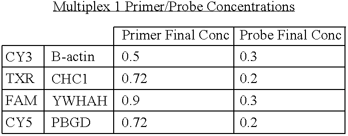

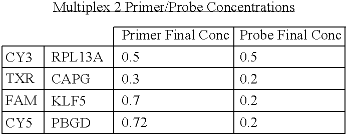

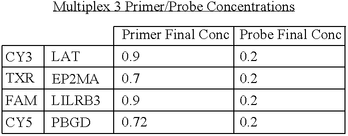



Experiment: Colon IVD primer Test
Purpose: To test the Internal BHQ primer and probe sets in the Cepheid system
Methods: Followed the above for assay set-up.

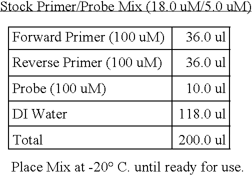

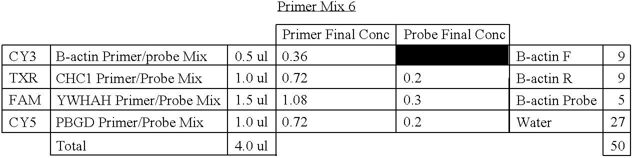

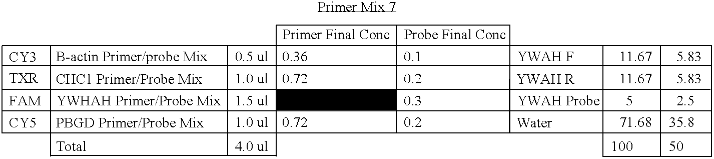

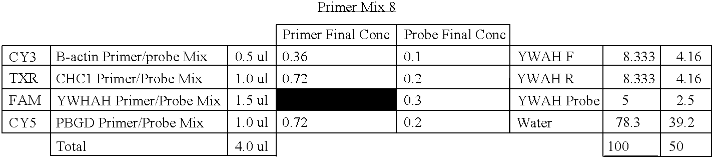

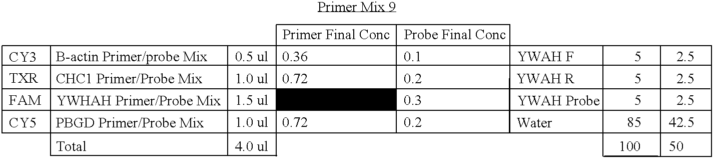

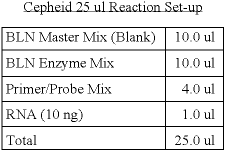






1. Combine all the reagents into a 25 ul Cepheid Tube
2. Before use, give the tubes a quick spin in a benchtop microcentrifuge.
3. Place the tubes into the Smartcycler and select CUP59 as the protocol

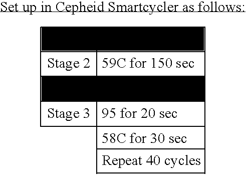

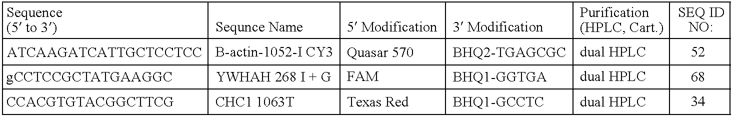

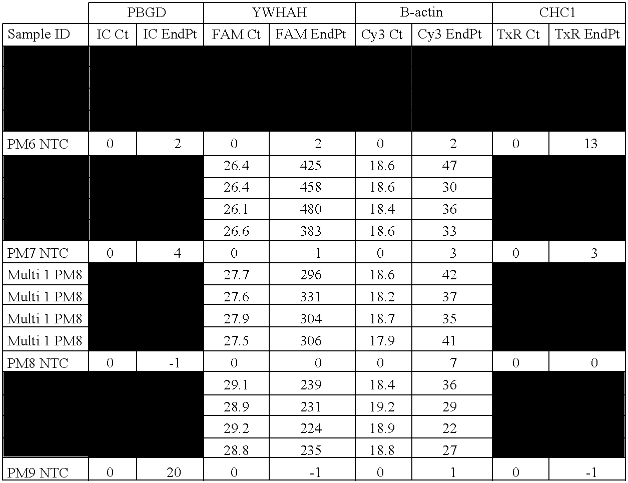



Experiment: Colon IVD primer Test
Methods: Followed the above for assay set-up.

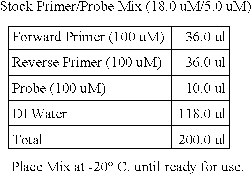

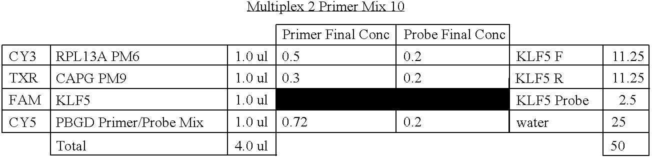

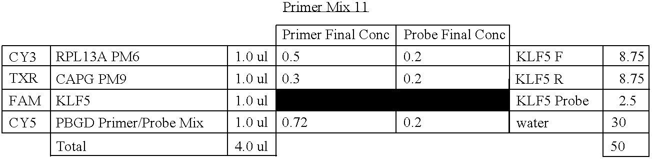

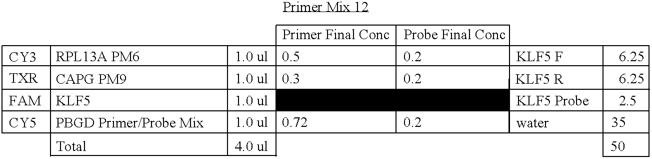

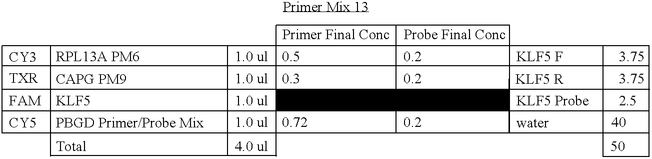

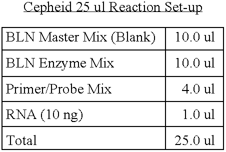






1. Combine all the reagents into a 25 ul Cepheid Tube
2. Before use, give the tubes a quick spin in a benchtop microcentrifuge.
3. Place the tubes into the Smartcycler and select Colon IVD 2 as the protocol

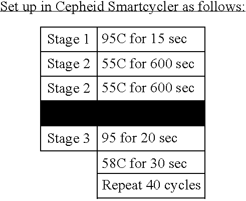

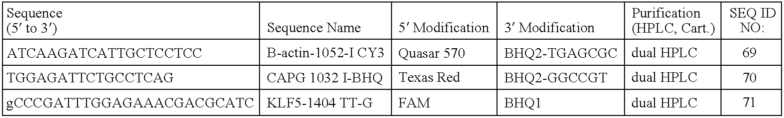

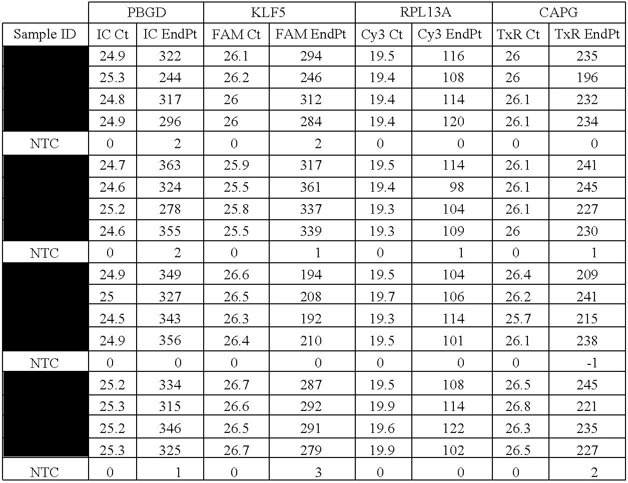



Experiment: Colon IVD primer Test
Methods: Followed the above for assay set-up.

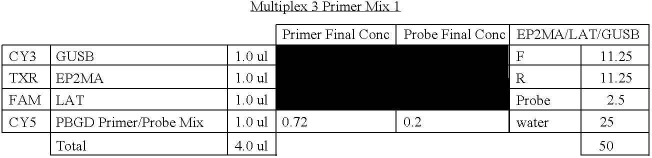

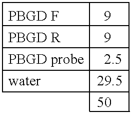

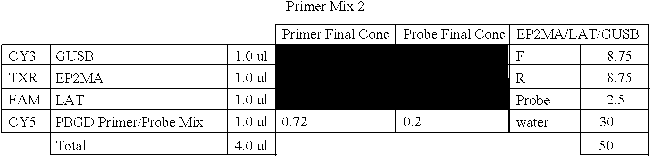

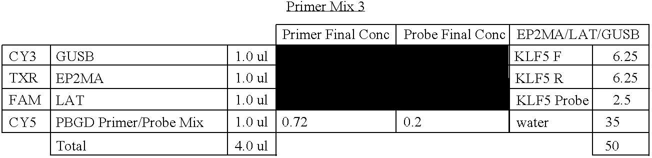

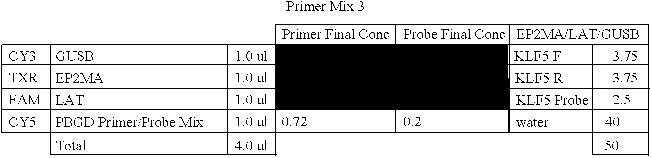

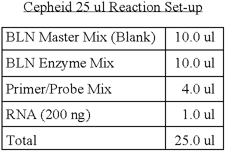






1. Combine all the reagents into a 25 ul Cepheid Tube
2. Before use, give the tubes a quick spin in a benchtop microcentrifuge.
3. Place the tubes into the Smartcycler and select Colon IVD 4c as the protocol

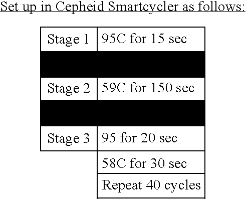

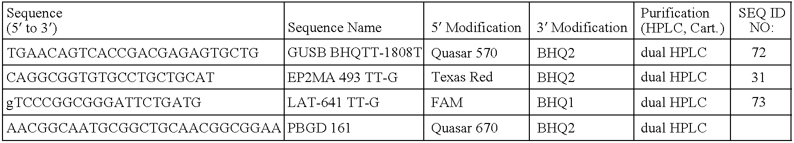

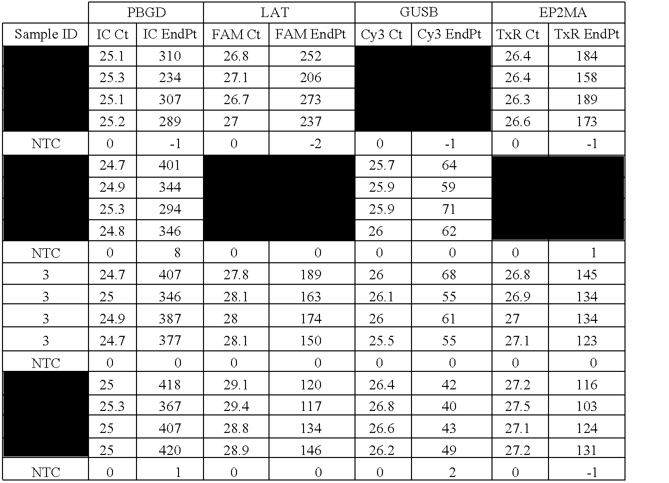



Experiment: Colon IVD primer Test
Methods: Followed the above for assay set-up.

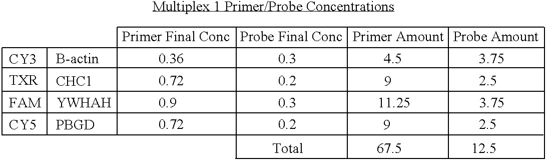

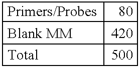

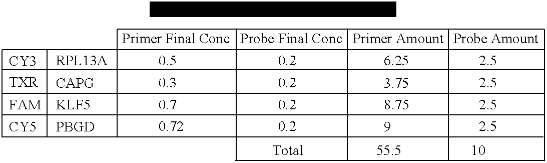

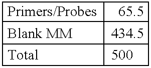

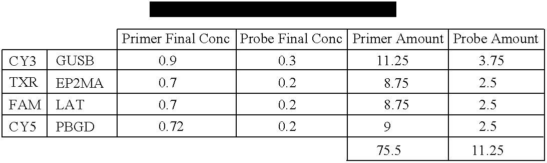

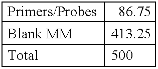

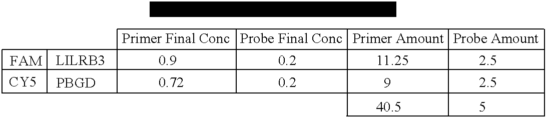

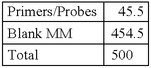

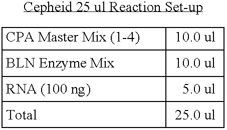









1. Combine all the reagents into a 25 ul Cepheid Tube
2. Before use, give the tubes a quick spin in a benchtop microcentrifuge.
3. Place the tubes into the Smartcycler and select Colon IVD 7a as the protocol

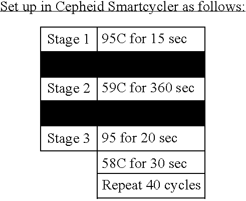

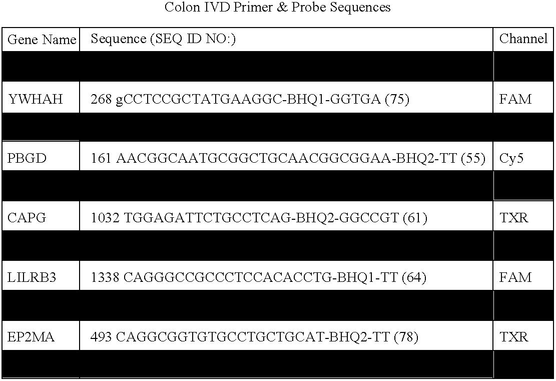

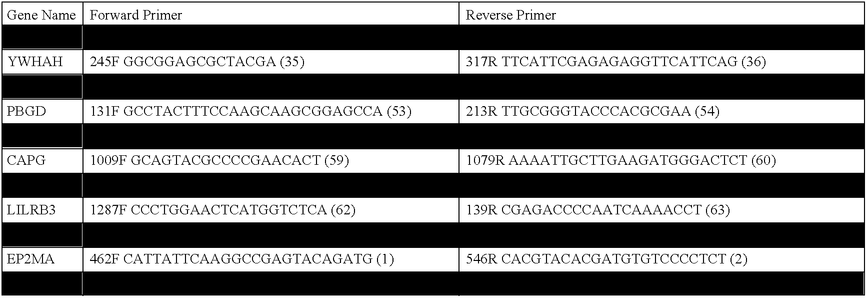

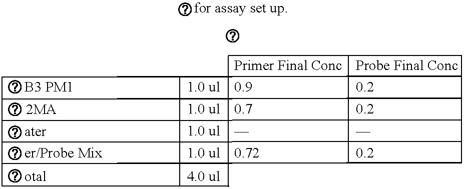

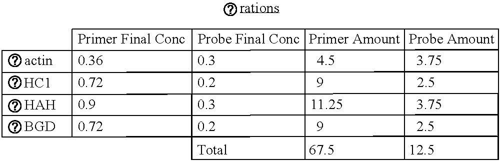

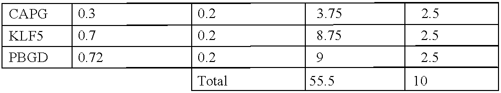






  65.5
434.5
500

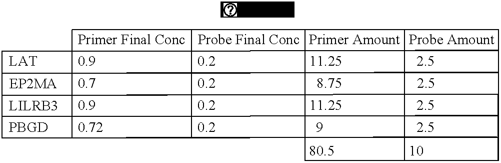

  90.5
409.5
500

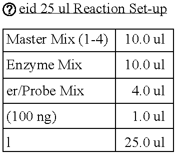

mbine all the reagents into a 25 ul Cepheid Tube
fore use, give the tubes a quick spin in a benchtop microcentrifuge.
ace the tubes into the Smartcycler and select Colon IVD 7a as the protocol

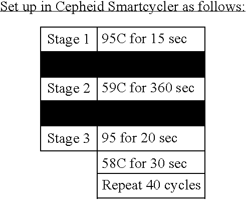

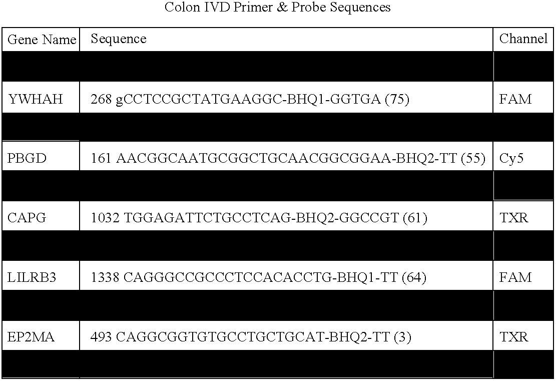

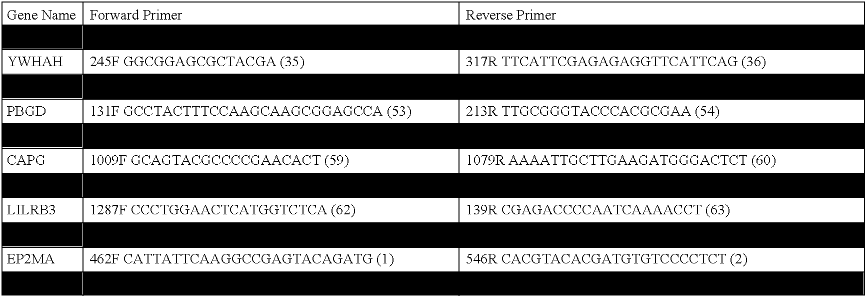



```

Colon IVD standard curves

| | | |
|---|---|---|
| 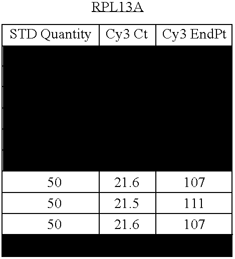 | 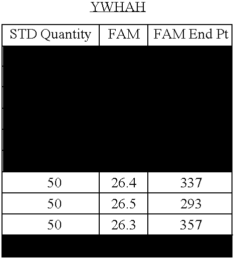 | 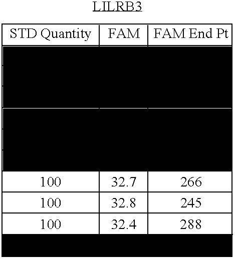 |
| 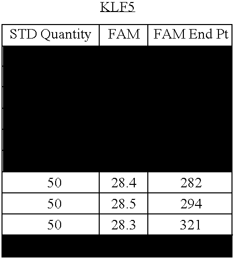 | 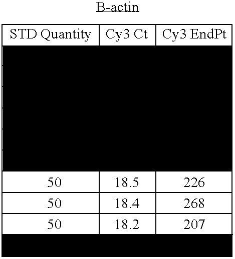 | 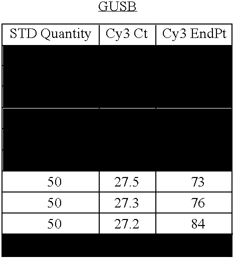 |
| 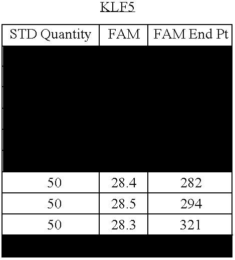 | 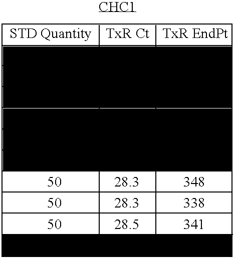 | 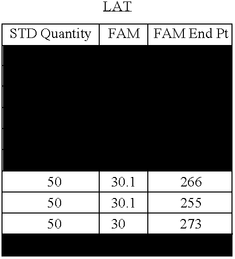 |
| 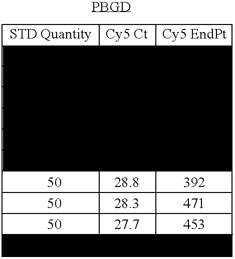 | 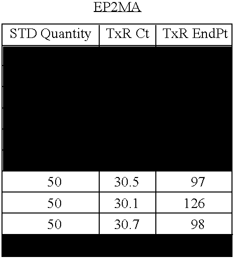 | |

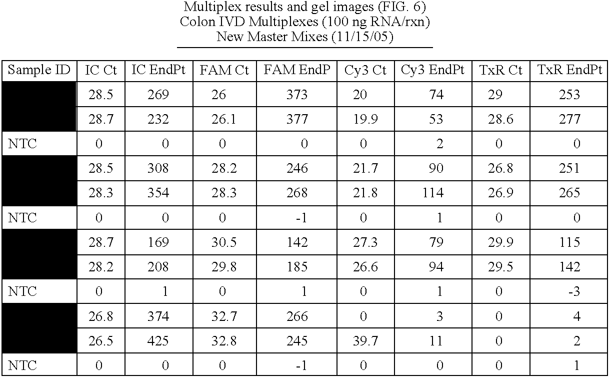

Expt: Colon IVD primer Test
Methods: Followed the above for assay set-up.

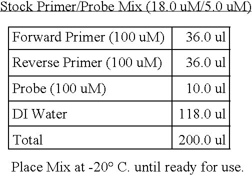

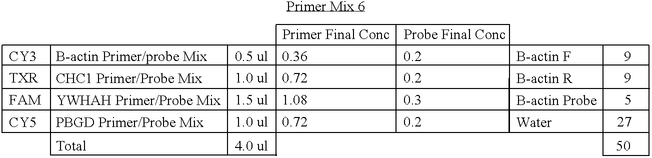

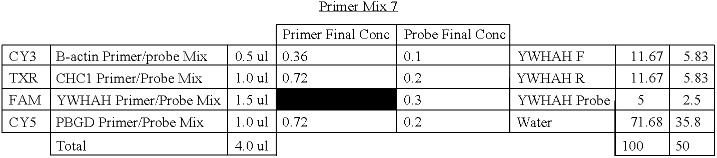

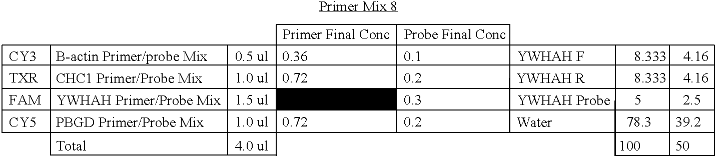

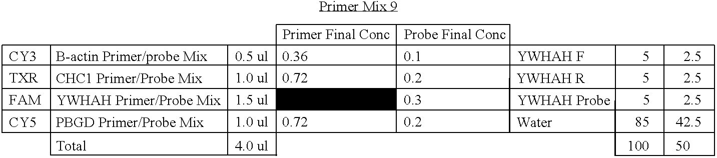

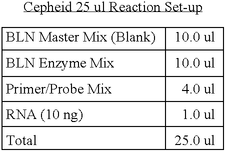

1. Combine all the reagents into a 25 ul Cepheid Tube
2. Before use, give the tubes a quick spin in a benchtop microcentrifuge.
3. Place the tubes into the Smartcycler and select CUP59 as the protocol

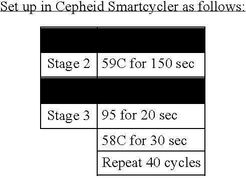

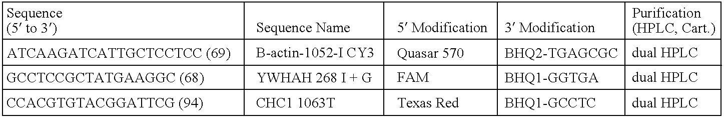

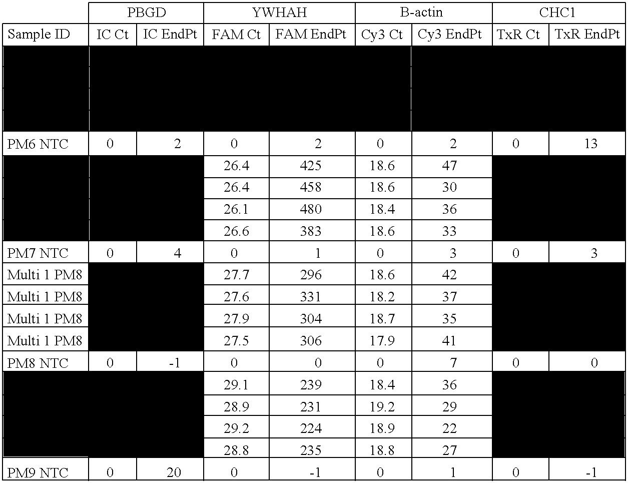

Experiment: Colon IVD primer Test
Methods: Followed the above for assay set-up.

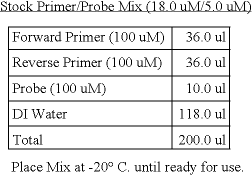

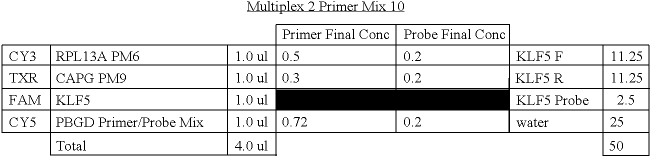

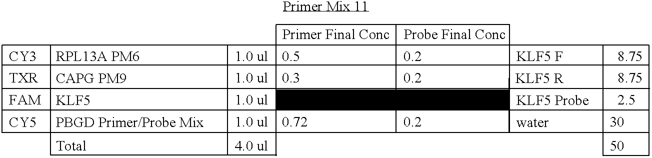

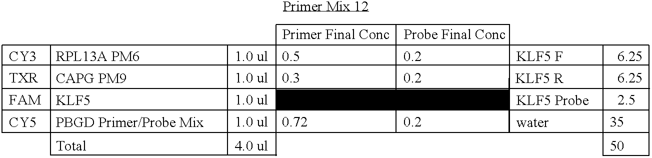

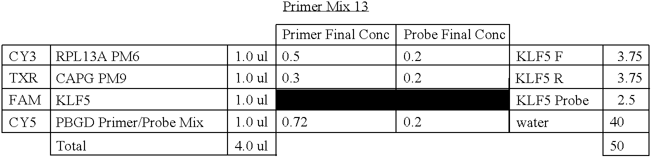

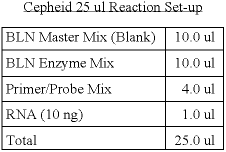

1. Combine all the reagents into a 25 ul Cepheid Tube
2. Before use, give the tubes a quick spin in a benchtop microcentrifuge.
3. Place the tubes into the Smartcycler and select Colon IVD 2 as the protocol

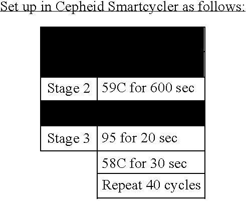

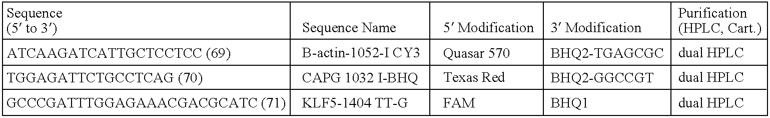

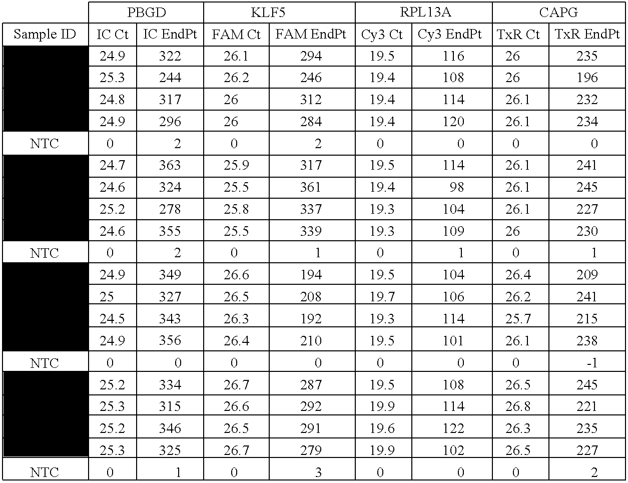

Experiment: Colon IVD primer Test
Methods: Followed the above for assay set-up.

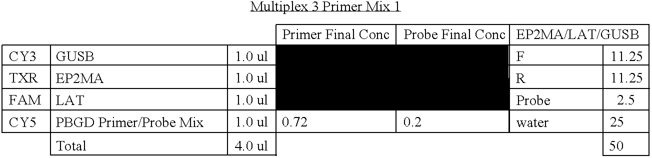

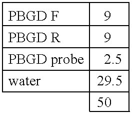

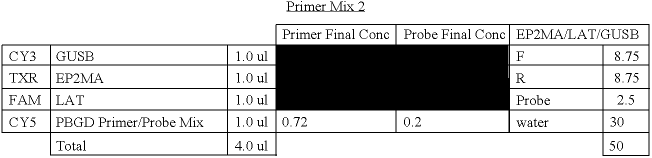

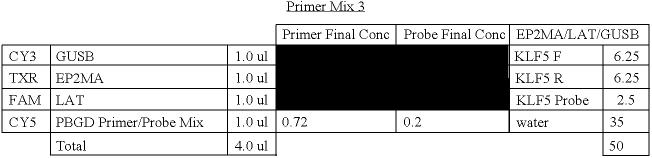

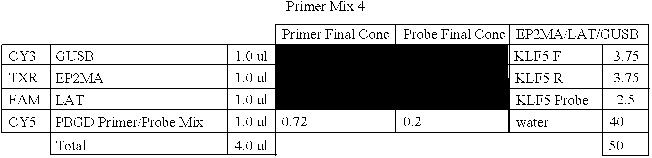

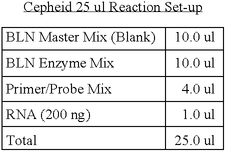

1. Combine all the reagents into a 25 ul Cepheid Tube
2. Before use, give the tubes a quick spin in a benchtop microcentrifuge.
3. Place the tubes into the Smartcycler and select Colon IVD 4c as the protocol

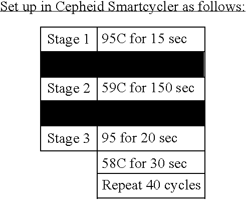

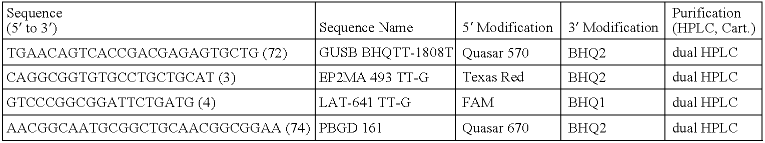

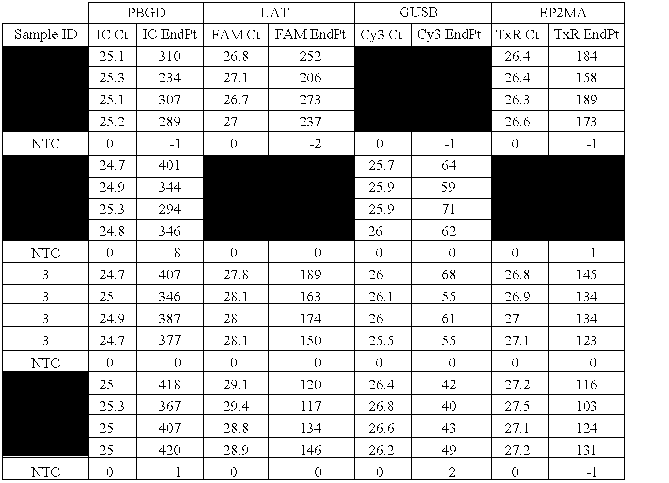

Experiment: Colon IVD primer Test
Methods: Followed the above for assay set-up.

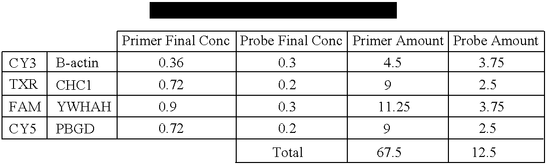

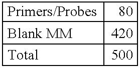

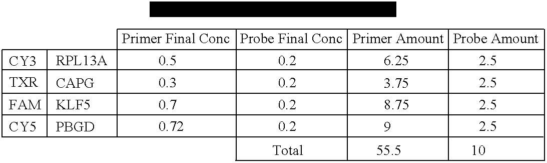

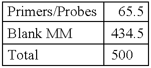

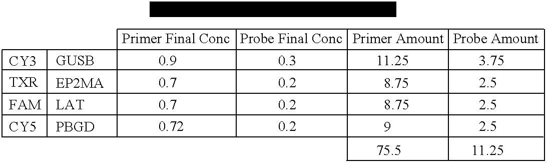

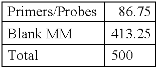

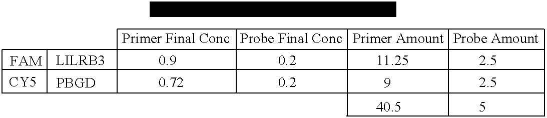

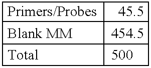

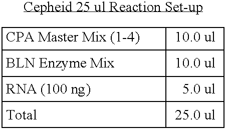

1. Combine all the reagents into a 25 ul Cepheid Tube
2. Before use, give the tubes a quick spin in a benchtop microcentrifuge.
3. Place the tubes into the Smartcycler and select Colon IVD 7a as the protocol

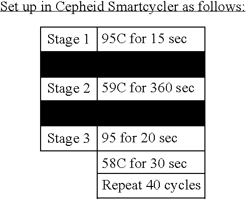

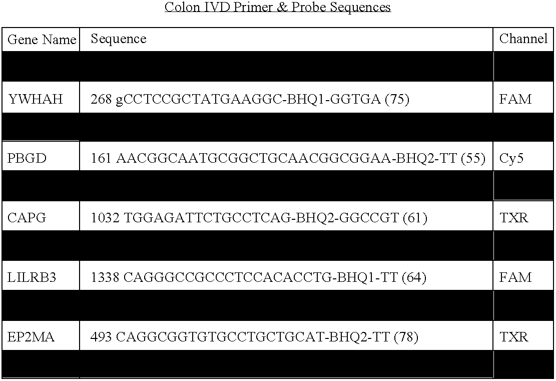

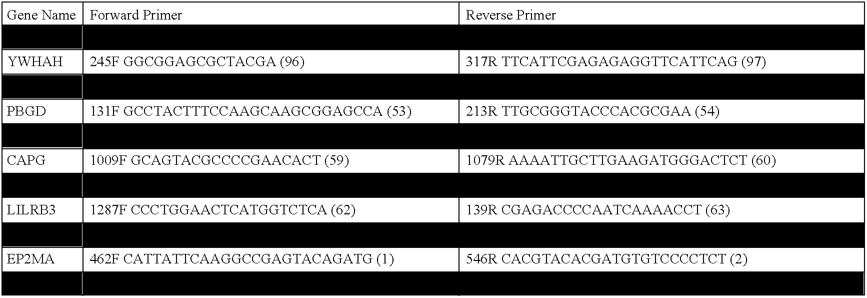

Colon IVD STD Curves

| | | |
|---|---|---|
| 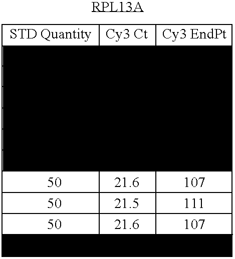 | 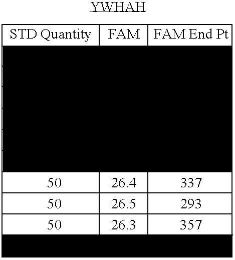 | 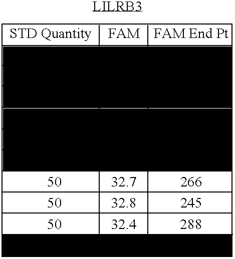 |
| 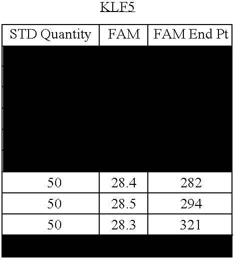 | 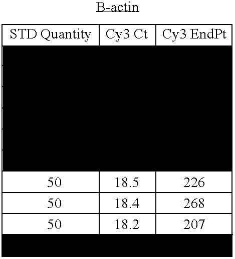 | 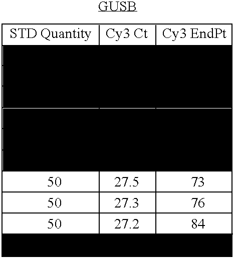 |
| 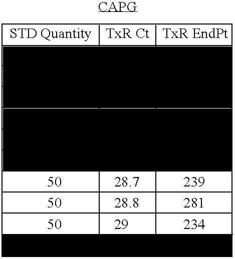 | 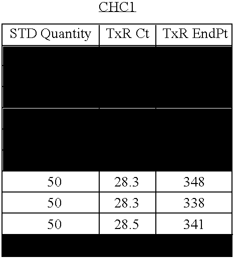 | 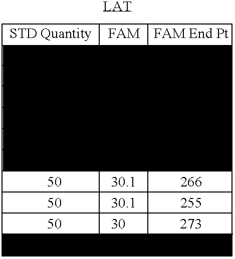 |
| 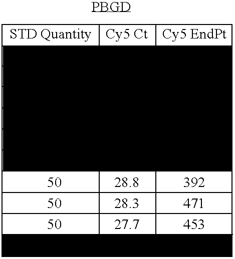 | 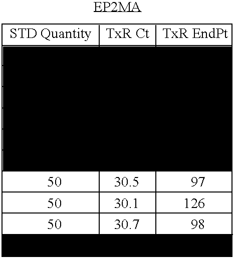 | |
| 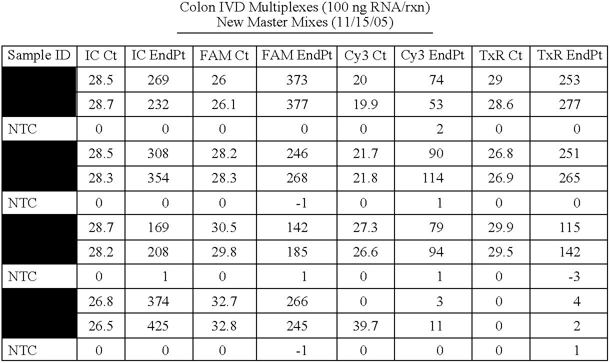 | | |

Experiment: Colon IVD primer Test
Methods: Followed the above for assay set-up.

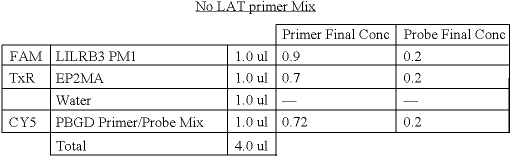

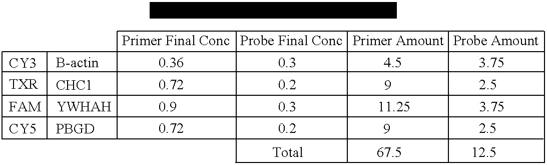

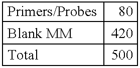

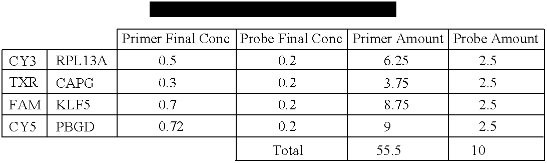

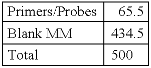

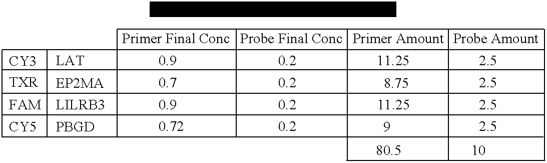

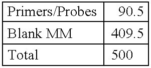

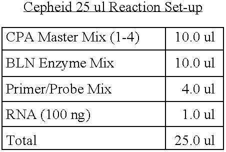

1. Combine all the reagents into a 25 ul Cepheid Tube
2. Before use, give the tubes a quick spin in a benchtop microcentrifuge.
3. Place the tubes into the Smartcycler and select Colon IVD 7a as the protocol

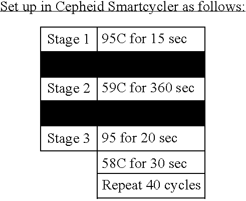

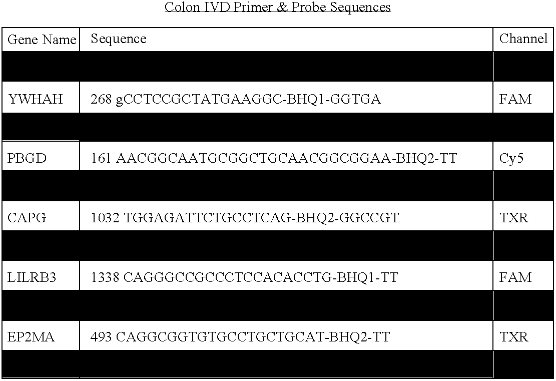

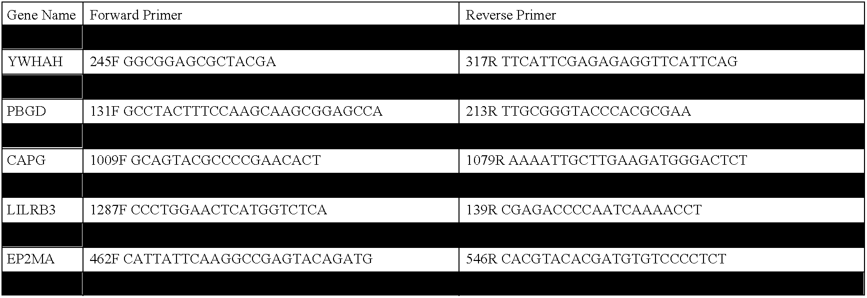

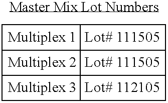

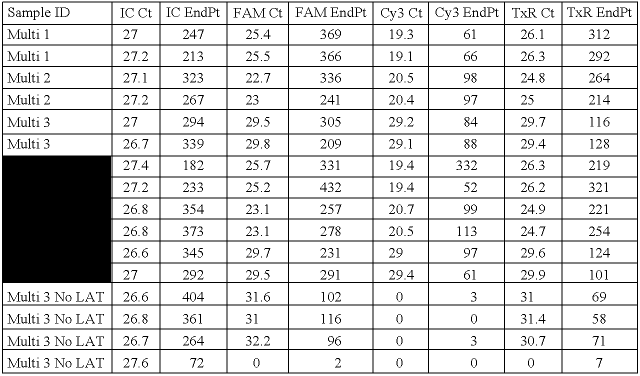

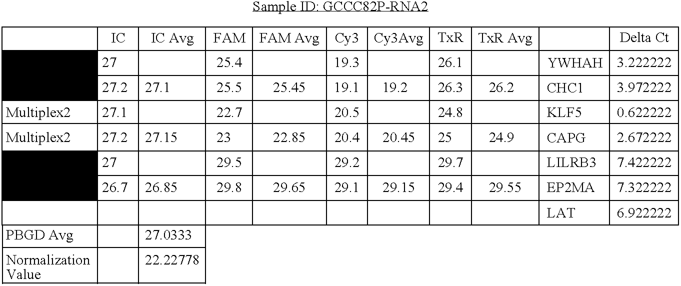

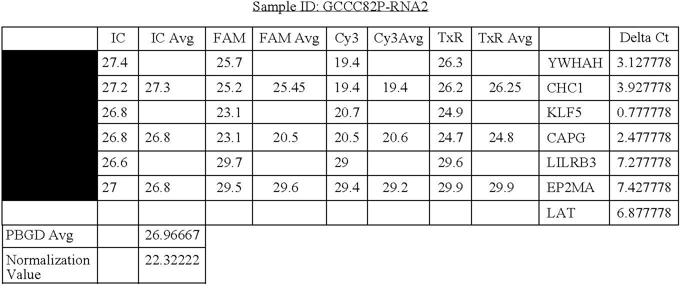

### REFERENCES

Allen et al. (2005a) Have we made progress in pharmacogenomics? The implementation of molecular markers in colon cancer Pharmacogenomics 6:603-614
Allen et al. (2005b) Role of genomic markers in colorectal cancer treatment J Clin Oncol 23:4545-4552
Beer et al. (2002) Gene expression profiles predict survival of patients with lung adenocarcinoma Nature Med 8:816-824
Compton et al. (2000) Prognostic factors in colorectal cancer. College of American Pathologists Consensus Statement 1999 Arch Pathol Lab Med 124:979-994
Golub et al. (1999) Molecular classification of cancer: class discovery and class prediction by gene expression monitoring Science 286:531-537
Halling et al. (1999) Microsatellite instability and 8p allelic imbalance in stage B2 and C colorectal cancers J Natl Cancer Inst 91:1295-1303
International multicenter pooled analysis of B2 colon cancer trials (IMPACT B2) investigators: Efficacy of adjuvant fluorouracil and folinic acid in B2 colon cancer J Clin Oncol 17:1356-1363 (1999)
Johnston (2005) Stage II colorectal cancer: to treat or not to treat Oncologist 10:332-334
Kaplan et al. (1958) Non-parametric estimation of incomplete observations J Am Stat Assoc 53:457-481
Liefers et al. (1998) Micrometastases and survival in stage II colorectal cancer N Engl J Med 339:223-228
Lipshutz et al. (1999) High density synthetic oligonucleotide arrays Nature Genet 21:20-24
Mamounas et al. (1999) Comparative efficacy of adjuvant chemotherapy in patients with Dukes' B versus Dukes° C. colon cancer: results from four National Surgical Adjuvant Breast and Bowel Project adjuvant studies (C-01, C-02, C-03, and C-04) J Clin Oncol 17:1349-1355
Markowitz et al. (2002) Focus on colon cancer Cancer Cell 1:233-236
Martinez-Lopez, et al. (1998) Allelic loss on chromosome 18q as a prognostic marker in stage II colorectal cancer Gastroenterology 114:1180-1187
McLeod et al. (1999) Tumor markers of prognosis in colorectal cancer Br J Cancer 79:191-203
Noura et al. (2002) Comparative detection of lymph node micrometastases of stage II colorectal cancer by reverse transcriptase polymerase chain reaction and immunohistochemistry J Clin Oncol 20:4232-4241
Ogunbiyi et al. (1998) Confirmation that chromosome 18q allelic loss in colon cancer is a prognostic indicator J Clin Oncol 16:427-433
Ramaswamy et al. (2001) Multiclass cancer diagnosis using tumor gene expression signatures Proc Natl Acad Sci USA 98:15149-15154
Ransohoff (2005) Bias as a threat to the validity of cancer molecular-marker research Nat Rev Cancer 5:142-149
Ratto et al. (1998) Prognostic factors in colorectal cancer. Literature review for clinical application D is Colon Rectum 41:1033-1049
Rosenwald et al. (2002) The use of molecular profiling to predict survival after chemotherapy for diffuse larger B-cell lymphoma N Engl J Med 346:1937-1947
Saltz et al. (1997) Adjuvant treatment of colorectal cance Annu Rev Med 48:191-202
Shibata et al. (1996) The DCC protein and prognosis in colorectal cancer N Engl J Med 335:1727-1732
Shipp et al. (2002) Diffuse large B-cell lymphoma outcome prediction by gene-expression profiling and supervised machine learning Nature Med 8:68-74
Simon et al. (2003) Pitfalls in the use of DNA microarray data for diagnostic and prognostic classification J Natl Cancer Inst 95:14-18
Su et al. (2001) Molecular classification of human carcinomas by use of gene expression signatures Cancer Res 61:7388-93
Sun et al. (1999) Expression of the deleted in colorectal cancer gene is related to prognosis in DNA diploid and low proliferative colorectal adenocarcinoma J Clin Oncol 17:1745-1750
Van de Vijver et al. (2002) A gene-expression signature as a predictor of survival in breast cancer N Engl J Med 347:1563-1575
van't Veer et al. (2002) Gene expression profiling predicts clinical outcome of breast cancer Nature 415:530-536.
Van't Veer et al. (2002) Gene expression profiling predicts clinical outcome of breast cancer. Nature 415:530-536
Wang et al (2005) Gene-expression profiles to predict distant metastasis of lymph-node-negative primary breast cancer Lancet 365:671-679
Wang et al. (2004) Gene expression profiles and molecular markers to predict recurrence of Dukes' B colon cancer J Clin Oncol 22:1564-1571
Watanabe et al. (2001) Molecular predictors of survival after adjuvant chemotherapy for colon cancer N Engl J Med 344:1196-1206
Wolmark et al. (1999) Clinical trial to assess the relative efficacy of fluorouracil and leucovorin, fluorouracil and levamisole, and fluorouracil, leucovorin, and levamisole in patients with Dukes' B and C carcinoma of the colon: results from National Surgical Adjuvant Breast and Bowel Project C-04 J Clin Oncol 17:3553-3559
Zhou et al. (2002) Counting alleles to predict recurrence of early-stage colorectal cancers Lancet 359:219-225

1. A method of determining predict recurrence of Dukes' B colon cancer comprising the steps of
a. obtaining a tumor sample from a patient; and
b. measuring the expression levels in the sample of genes selected from the group consisting of those encoding mRNA:
i. corresponding to SEQ ID Nos: 7-28; or
ii. recognized by the primer and/or probe corresponding to at least one of SEQ ID Nos 29-79 and 94-97; or
iii. identified by the production of at least one of the amplicons selected from SEQ ID NOs: 5-6, 80-93
wherein the gene expression levels above or below pre-determined cut-off levels are indicative of predict recurrence of Dukes' B colon cancer.

2. A method of determining patient treatment protocol comprising the steps of
a. obtaining a tumor sample from a patient; and
b. measuring the expression levels in the sample of genes selected from the group consisting of those encoding mRNA:
i. corresponding to SEQ ID Nos: 7-28; or
ii. recognized by the primer and/or probe corresponding to at least one of SEQ ID Nos 29-79 and 94-97; or
iii. identified by the production of at least one of the amplicons selected from SEQ ID NOs: 5-6, 80-93
wherein the gene expression levels above or below pre-determined cut-off levels are sufficiently indicative of risk of recurrence to enable a physician to determine the degree and type of therapy recommended to prevent recurrence.

3. A method of determining patient treatment protocol comprising the steps of
a. obtaining a tumor sample from a patient; and
b. measuring the expression levels in the sample of genes selected from the group consisting of those encoding mRNA:
i. corresponding to SEQ ID Nos: 7-28; or
ii. recognized by the primer and/or probe corresponding to at least one of SEQ ID Nos 29-79 and 94-97; or
iii. identified by the production of at least one of the amplicons selected from SEQ ID NOs: 5-6, 80-93
wherein the gene expression levels above or below pre-determined cut-off levels are sufficiently indicative of risk of recurrence to enable a physician to determine the degree and type of therapy recommended to prevent recurrence.

4. A method of treating a patient comprising the steps of:
a. obtaining a tumor sample from a patient; and
b. measuring the expression levels in the sample of genes selected from the group consisting of those encoding mRNA:
i. corresponding to SEQ ID Nos: 7-28; or
ii. recognized by the primer and/or probe corresponding to at least one of SEQ ID Nos 29-79 and 94-97; or
iii. identified by the production of at least one of the amplicons selected from SEQ ID NOs: 5-6, 80-93 and;
c. treating the patient with adjuvant therapy if they are a high risk patient.

5. A method of treating a patient comprising the steps of:
a. obtaining a tumor sample from a patient; and
b. measuring the expression levels in the sample of genes selected from the group consisting of those encoding mRNA:
i. corresponding to SEQ ID Nos: 7-28; or
ii. recognized by the primer and/or probe corresponding to at least one of SEQ ID Nos 29-79 and 94-97; or
iii. identified by the production of at least one of the amplicons selected from SEQ ID NOs: 5-6, 80-93 and;
c. treating the patient with adjuvant therapy if they are a high risk patient.

6. The method of any one of claims 1-5 wherein the sample is obtained from a primary tumor.

7. The method of claim 1, 2 or 4 wherein the preparation is obtained from a biopsy or a surgical specimen.

8. The method of any one of claims 1-5 further comprising measuring the expression level of at least one gene constitutively expressed in the sample.

9. The method of any one of claims 1-5 wherein the specificity is at least about 40%.

10. The method of any one of claims 1-5 wherein the sensitivity is at least at least about 90%.

11. The method of any one of claims 1-5 wherein the expression pattern of the genes is compared to an expression pattern indicative of a relapse patient.

12. The method of claim 11 wherein the comparison of expression patterns is conducted with pattern recognition methods.

13. The method of claim 12 wherein the pattern recognition methods include the use of a Cox's proportional hazards analysis.

14. The method of any one of claims 1-5 wherein the pre-determined cut-off levels are at least 1.5-fold over- or under-expression in the sample relative to benign cells or normal tissue.

15. The method of any one of claims 1-5 wherein the pre-determined cut-off levels have at least a statistically significant p-value over- or under-expression in the sample having metastatic cells relative to benign cells or normal tissue.

16. The method of claim 15 wherein the p-value is less than 0.05.

17. The method of any one of claims 1-5 wherein gene expression is measured on a microarray or gene chip.

18. The method of claim 17 wherein the microarray is a cDNA array or an oligonucleotide array.

19. The method of claim 18 wherein the microarray or gene chip further comprises one or more internal control reagents.

20. The method of any one of claims 1-5 wherein gene expression is determined by nucleic acid amplification conducted by polymerase chain reaction (PCR) of RNA extracted from the sample.

21. The method of claim 20 wherein said PCR is reverse transcription polymerase chain reaction (RT-PCR).

22. The method of claim 21, wherein the RT-PCR further comprises one or more internal control reagents.

23. The method of any one of claims 1-5 wherein gene expression is detected by measuring or detecting a protein encoded by the gene.

24. The method of claim 23 wherein the protein is detected by an antibody specific to the protein.

25. The method of any one of claims 1-5 wherein gene expression is detected by measuring a characteristic of the gene.

26. The method of claim 25 wherein the characteristic measured is selected from the group consisting of DNA amplification, methylation, mutation and allelic variation.

27. A composition comprising at least one probe set selected from the group consisting of the SEQ ID NOs: 29-79.

28. A kit for conducting an assay to determine predict recurrence of Dukes' B colon cancer a biological sample comprising: materials for detecting isolated nucleic acid sequences, their complements, or portions thereof of a combination of genes selected from the group consisting of those encoding mRNA corresponding to the SEQ ID NOs: 7-28.

29. The kit of claim 28 further comprising reagents for conducting a microarray analysis.

30. The kit of claim 28 further comprising a medium through which said nucleic acid sequences, their complements, or portions thereof are assayed.

31. Articles for assessing status comprising: materials for detecting isolated nucleic acid sequences, their complements, or portions thereof of a combination of genes selected from the group consisting of those encoding mRNA corresponding to the SEQ ID NOs: 7-28

32. The articles of claim 31 further comprising reagents for conducting a microarray analysis.

33. The articles of claim 31 further comprising a medium through which said nucleic acid sequences, their complements, or portions thereof are assayed.

34. A microarray or gene chip for performing the method of any one of claims 1-5.

35. The microarray of claim 34 comprising isolated nucleic acid sequences, their complements, or portions thereof of a combination of genes selected from the group consisting of those encoding mRNA corresponding to the SEQ ID NOs: 7-28.

36. The microarray of claim 35 wherein the sequences are selected from SEQ ID NOs: 29-79 and 94-97.

37. The microarray of claim 35 comprising a cDNA array or an oligonucleotide array.

38. The microarray of claim 35 further comprising or more internal control reagents.

39. A diagnostic/prognostic portfolio comprising isolated nucleic acid sequences, their complements, or portions thereof of a combination of genes selected from the group consisting of those encoding mRNA corresponding to the SEQ ID NOs: 7-28.

40. The portfolio of claim 39 wherein the sequences are selected from SEQ ID NOs: 29-79 and 94-97.
